SEQUENCE LISTING (150 sequences)

SEQ ID NO: 1 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 2 - protein, 512 aa, Escherichia coli
MTNNPPSAQIKPGEYGFPLKLKARYDNFIGGEWVAPADGEYYQNLTPVTGQLLCEVASSGKRDIDLALDAAHKVKDKWAHTSVQDRAAILFKIADRMEQNLELLATAETWDNGKPIRETSAADVPLAIDHFRYFASCIRAQEGGISEVDSETVAYHFHEPLGVVGQIIPWNFPLLMASWKMAPALAAGNCVVLKPARLTPLSVLLLMEIVGDLLPPGVVNVVNGAGGVIGEYLATSKRIAKVAFTGSTEVGQQIMQYATQNIIPVTLELGGKSPNIFFADVMDEEDAFFDKALEGFALFAFNQGEVCTCPSRALVQESIYERFMERAIRRVESIRSGNPLDSVTQMGAQVSHGQLETILNYIDIGKKEGADVLTGGRRKLLEGELKDGYYLEPTILFGQNNMRVFQEEIFGPVLAVTTFKTMEEALELANDTQYGLGAGVWSRNGNLAYKMGRGIQAGRVWTNCYHAYPAHAAFGGYKQSGIGRETHKMMLEHYQQTKCLLVSYSDKPLGLF

SEQ ID NO: 3 - protein, 495 aa, Escherichia coli
MNFHHLAYWQDKALSLAIENRLFINGEYTAAAENETFETVDPVTQAPLAKIARGKSVDIDRAMSAARGVFERGDWSLSSPAKRKAVLNKLADLMEAHAEELALLETLDTGKPIRHSLRDDIPGAARAIRWYAEAIDKVYGEVATTSSHELAMIVREPVGVIAAIVPWNFPLLLTCWKLGPALAAGNSVILKPSEKSPLSAIRLAGLAKEAGLPDGVLNVVTGFGHEAGQALSRHNDIDAIAFTGSTRTGKQLLKDAGDSNMKRVWLEAGGKSANIVFADCPDLQQAASATAAGIFYNQGQVCIAGTRLLLEESIADEFLALLKQQAQNWQPGHPLDPATTMGTLIDCAHADSVHSFIREGESKGQLLLDGRNAGLAAAIGPTIFVDVDPNASLSREEIFGPVLVVTRFTSEEQALQLANDSQYGLGAAVWTRDLSRAHRMSRRLKAGSVFVNNYNDGDMTVPFGGYKQSGNGRDKSLHALEKFTELKTIWISLEA

SEQ ID NO: 4 - protein, 482 aa, Escherichia coli
MKLNDSNLFRQQALINGEWLDANNGEAIDVTNPANGDKLGSVPKMGADETRAAIDAANRALPAWRALTAKERATILRNWFNLMMEHQDDLARLMTLEQGKPLAEAKGEISYAASFIEWFAEEGKRIYGDTIPGHQADKRLIVIKQPIGVTAAITPWNFPAAMITRKAGPALAAGCTMVLKPASQTPFSALALAELAIRAGVPAGVFNVVTGSAGAVGNELTSNPLVRKLSFTGSTEIGRQLMEQCAKDIKKVSLELGGNAPFIVFDDADLDKAVEGALASKFRNAGQTCVCANRLYVQDGVYDRFAEKLQQAVSKLHIGDGLDNGVTIGPLIDEKAVAKVEEHIADALEKGARVVCGGKAHERGGNFFQPTILVDVPANAKVSKEETFGPLAPLFRFKDEADVIAQANDTEFGLAAYFYARDLSRVFRVGEALEYGIVGINTGIISNEVAPFGGIKASGLGREGSKYGIEDYLEIKYMCIGL

SEQ ID NO: 5 - protein, 481 aa, Burkholderia ambifaria
MANVTYTDTQLLIDGEWVDAASGKTIDVVNPATGKAIGKVAHAGIADLDRALAAAQRGFEAWRKVPANERAATMRKAAALVRERADAIAQLMTQEQGKPLTEARVEVLSAADIIEWFADEGRRVYGRIVPPRNLGAQQMVVKEPVGPVAAFTPWNFPVNQVVRKLSAALATGCSFLVKAPEETPASPAALLRAFVDAGVPAGVIGLVYGEPAEISSYLIAHPVIRKVTFTGSTPVGKQLAALAGQHMKRATMELGGHAPVIVAEDADVALAVKAAGGAKFRNAGQVCISPTRFLVHNSIRDEFTRALVQHAEGLKIGNGLEEGTTLGALANPRRLTAMASVVENARKVGASIETGGERIGSEGNFFAPTVIANVPLEADVFNNEPFGPVAAIRGFDKLEDAIAEANRLPFGLAGYAFTRSFANVHLLSQRLEVGMLWINQPATPWPEMPFGGVKDSGYGSEGGPEALEPYLVTKSVTVMAV

SEQ ID NO: 6 - protein, 525 aa, Pseudomonas putida
MPLTGNLLIGQRPVTGSRDAIRAIDPTTGQTLEPAYLGGTGEHVAQACALAWAAFDAYRETSLEQRAEFLEAIATQIEALGDALIDRAVIETGLPKARIQGERGRTCTQLRTFARTVRAGEWLDVRIDSALPERQPLPRADLRQRQVALGPVAVFGASNFPLAFSVAGGDTASALAAGCPVVVKAHSAHPGTSELVGQAVAQAVKQCGLPEGVFSLLYGSGREVGIALVSDPRIKAVGFTGSRSGGMALCQAAQARPEPIPVYAEMSSINPVFLFDAALQARAEALAQGFVASLTQGAGQFCTNPGLVIARQGPALQRFITAAAGYVQQGAAQTMLTPGIFSAYQAGIAALADNPHAQAITSGQAGQGPNQCQAQLFVTQAEAFLADPALQAEVFGAASLVVACTDDEQVRQVAEHLEGQLTATLQLDEADIDSARALLPTLERKAGRILVNGWPTGVEVCDAMVHGGPFPATSDARTTSVGTAAILRFLRPVCYQDVPDALLPQALKHGNPLQLRRLLDGKRED

SEQ ID NO: 7 - protein, 474 aa, Escherichia coli
MQHKLLINGELVSGEGEKQPVYNPATGDVLLEIAEASAEQVDAAVRAADAAFAEWGQTTPKVRAECLLKLADVIEENGQVFAELESRNCGKPLHSAFNDEIPAIVDVFRFFAGAARCLNGLAAGEYLEGHTSMIRRDPLGVVASIAPWNYPLMMAAWKLAPALAAGNCVVLKPSEITPLTALKLAELAKDIFPAGVINILFGRGKTVGDPLTGHPKVRMVSLTGSIATGEHIISHTASSIKRTHMELGGKAPVIVFDDADIEAVVEGVRTFGYYNAGQDCTAACRIYAQKGIYDTLVEKLGAAVATLKSGAPDDESTELGPLSSLAHLERVGKAVEEAKATGHIKVITGGEKRKGNGYYYAPTLLAGALQDDAIVQKEVFGPVVSVTPFDNEEQVVNWANDSQYGLASSVWTKDVGRAHRVSARLQYGCTWVNTHFMLVSEMPHGGQKLSGYGKDMSLYGLEDYTVVRHVMVKH

SEQ ID NO: 8 - protein, 500 aa, Escherichia coli
MMTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 9 - protein, 499 aa, Escherichia coli; identical to SEQ ID NO: 1

SEQ ID NO: 10 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIVGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 11 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPVVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 12 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPFPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADHLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 13 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 14 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLEPLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCGKVCSFLDDAQAQQAELIRGSNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 15 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGSVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCDKVRSFLDDAQAQQAELIRGASGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDGWCETKSVCVRY

SEQ ID NO: 16 - protein, 499 aa, Escherichia coli
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHVAKISFTGSTATGKGIARTAADRLTRVTLELGGKNPAIVLKDADPQWVIEGLMTGSFLNQGQVCAASSRIYIEAPLFDTLVSGFEQAVKSLQVGPGMSPVAQINPLVSRAHCDKVCSFLDDAQAQQAELIRGLNGPAGEGYYVAPTLVVNPDAKLRLTREEVFGPVVNLVRVADGEEALQLANDTEYGLTASVWTQNLSQALEYSDRLQAGTVWVNSHTLIDANLPFGGMKQSGTGRDFGPDWLDSWRETKSVCVRY

SEQ ID NO: 17 - protein, 499 aa, Escherichia coli; identical to SEQ ID NO: 16

SEQ ID NO: 18 - protein, 499 aa, Escherichia fergusonii
MTEPHVAVLSQVQQFLDRQHGLYIDGRPGPAQSEKRLAIFDPATGQEIASTADANEADVDNAVMSAWRAFVSRRWAGRLPAERERILLRFADLVEQHSEELAQLETMEQGKSIAISRAFEVGCTLNWMRYTAGLTTKIAGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHIAKISFTGSTATGKGIARTAADRLTRVTLELGGKNPAIVLKDADPQWVIEGLMTGSFLNQGQVCAASSRIYIEAPLFDNLVSGFEQAVKSLQVGPGMSPVAQINPLVSRAHCDKVRSFLDDAQAQKAELIRGASGPAGEGYYVAPTLVVNPDAKLRLTREEVFGPVVNLVRVADGEEALQLANDTEYGLTASVWTQNLTQALEYSDRLQAGTVWVNSHTLIDANLPFGGMKQSGTGRDFGPDWLDGWCETKSVCVRY

SEQ ID NO: 19 - protein, 499 aa, Escherichia fergusonii
MTEPHVAVLSQVQQFLDRQHGLYIDGRPGPAQSEKRLPIFNPATGQEIASTADANEADVDNAVMSAWRAFVSRRWAGRLPAERERILLRFADLVEQHSEELAQLETLEQGKSIAISRAFEVGCTLNWMRYTAGLTTKIAGKTLDLSIPLPQGARYQVWTRKEPVGVVAGIVPWNFPLMIGMWKVMPALAAGCSIVIKPSETTPLTMLRVAELASEAGIPDGVFNVVTGAGAVCGAALTSHPHVAKISFTGSTATGKGIARTAADRLTRVTLELGGKNPAIVLKDADPQWVIEGLMTGSFLNQGQVCAASLRIYIEAPLFDTLVSGFEQAVKSLQVGPGMSPVAQINPLVSRAHCDKVCSFLDDAQAQQAELIRGSNGPAGEGYYVAPTLVVNPDAKLRLTREEVFGPVVNLVRVADGEEALQLANDTEYDLTASVWTQNLSRALEYSDRLQAGTVWVNSHTLIDANLPFGGMKQSGTGRDFGPDWLDGWCETKSVCVRY

SEQ ID NO: 20 - protein, 499 aa, Enterobacter cloacae
MSESQVAIQPGVQQFLDRHHGLWIEGRQAASESEKRLNIYNPATGEVIASTADASVDDVDRAVMSGWRAFVARNWAGKLPAERERILLRFADLVEQHSEELAQLETLEQGKSINISRLFEVGCTLNWMRYTAGLTTKITGKTLDLSIPLPQGARYQAWTRKEPVGVVAGIVPWNFPLMIGMWKVMPALAAGCSIVIKPSETTPLTMLRVAELASEAGIPDGVFNVVTGSGAVCGAALTSHPHIAKVSFTGSTATGKQIARAAADTLTGVTLELGGKNPAIVLKDADPAWVIEGLMTGSFLNQGQVCAASSRIYIEAPLFDTLVSGFEQAVKSLSVGPGMSPEAFINPLVSRAHCDKVQTFLDEAKSRNAELITGNRGPDGKGYYISPTLVVNPDPGLRLTREEVFGPVVNLVRVADGEEALTLANDTEYGLTASVWTQNISKALEYTDRLQAGTVWVNSHTLIDANLPFGGMKQSGTGRDFGPDWLDGWCETKSVCVRY

SEQ ID NO: 21 - protein, 519 aa, Enterobacter cancerogenus
MAKPLLDCINILRRITKEYPMSESQVAVLPCVQQFLDRHHGLWIEGREVASDGEKRLNVYNPATGEVIASTADASVDDVDRAVMSGWRAFVSRSWAGTLPAERERILLHFADLVEQHGEELAQLETLEQGKSINISRAFEVGCTLNWMRYTAGLTTKIAGKTLDLSIPLPQGARYQAWTRKEPVGVVAGIVPWNFPLMIGMWKVMPALAAGCSIVIKPSETTPLTLLRVAELASEAGIPDGVFNVITGSGATCGAALTSHPRIAKVSFTGSTATGKQIARTAAETLTGVTLELGGKNPAIVLKDADPAWVIEGLMTGSFLNQGQVCAASSRIYIEAPLFDTLVSGFEQAVKSLSVGPGMSPQAFINPLVSRAHCDKVQTFLDEAASRKAELISGSRGPDGKGYYVSPTLVVNPDASLRLTREEVFGPVVNLVRVADGEEALRLANDTEYGLTASVWTQNISKALEYTDRLQAGTVWVNSHTLIDANLPFGGMKQSGTGRDFGPDWLDGWCETKSVCVRY

SEQ ID NO: 22 - protein, 499 aa, Klebsiella pneumonia
MSTSQIALLASVQQFLDRQHGLYIDGAPCAAQSENRLTVWDPATGQAIATTADASPADVDRAVMSAWRAFVDRRWAGRTPADRERILLRFADLVEQHGEELAQLETLEQGKSIAISRAFEVGCTLNWMRYTAGLTTKISGRTLDVSIPFPQGARYQAWTKKEPVGVVAGIVPWNFPLMIGMWKVMPALAAGCSIVIKPSETTPLTLLRVAELATQAGIPDGVFNVVTGSGAGCGAALTAHPQVAKVSFTGSTATGKQIARVAADRLTRVTLELGGKNPAIVLKDADPQWVIEGLMTGSFLNQGQVCAASSRIYIEAPLFDTLVSGFEQAVKSLQVGPGMQETAQINPVVSRAHCDKVAAYLEEARQQKAELISGSAGPDAGGYYIPPTLVVNPDAGLRLSREEVFGPVVNLVRVADGEEALRLANDSDFGLTASVWTRDLTQALNYTDRLQAGTVWVNSHTLIDANLPFGGMKQSGTGRDFGPDWLDGWCETKSVCVRY

SEQ ID NO: 23 - protein, 499 aa, Enterobacter cloacae
MSDSQVAILERVQQFLARQHGLFIDGAQQPSRSDKRLTVWNPATGEAIATTADANSADVDSAVMSAWRAFVDRRWAGRLPAERERILLRFTDLVEQHSEELAQLETLEQGKSINISRAFEVACTLNWMRYTAGLTTKITGQTLDVSIPLPPGARYQAWTRKEPVGVVAGIVPWNFPLMIGMWKVMPALAAGCSIVIKPSETTPLTLLRVAELAVEAGIPEGVFNVVTGSGAECGAALTSHPHVAKVSFTGSTQTGKQIARTAADRLTRVTLELGGKNPAIVLKDADPQWVIEGLMMGSFLNQGQVCAASSRIYIEAPLFDTLVTGFEQAVKSLSVGPGMSEAAQINPLASRAHCDKVAAFLHDAQQHHAELIHGNEGPGGQGYYIRPTLVVNPDARLRLTREEVFGPVVNLVRVADGEEALRLANETEYGLTASVWTRDLSQALGYSDRLQAGTVWVNSHTLIDPNLPFGGMKQSGTGRDFGTDWLDGWCETKSVCVRY

SEQ ID NO: 24 - protein, 499 aa, Klebsiella pneumonia
MSTSQIALLASVQQFLDRQHGLYIDGAPCAAQSENRLTVWDPATGQAIATTADASPADVDRAVMSAWRAFVDRRWAGRTPADRERILLRFADLVEQHGEELAQLETLEQGKSIAISRAFEVGCTLNWMRYTAGLTTKISGRTLDVSIPFPQGARYQAWTKKEPVGVVAGIVPWNFPLMIGMWKVMPALAAGCSIVIKPSETTPLTLLRVAELATLAGIPDGVFNVVTGSGAGCGAALTAHPQVAKVSFTGSTATGKQIARVAADRLTRVTLELGGKNPAIVLKDADPQWVIEGLMTGSFLNQGQVCAASSRIYIEAPLFDTLVSGFEQAVKSLQVGPGMQETAQINPVVSRAHCDKVAAYLEEARQQKAELISGSAGPDAGGYYIPPTLVVNPDAGLRLTREEVFGPVVNLVRVADGEEALRLANDSDFGLTASVWTRDLTQALNYTDRLQAGTVWVNSHTLIDANLPFGGMKQSGTGRDFGPDWLDGWCETKSVCVRY

SEQ ID NO: 25 - protein, 499 aa, Klebsiella pneumonia
MSTSQIALLASVQQFLDRQHGLYIDGAPCAAQSENRLTVWDPATGQAIATTADASPADVDRAVMSAWRAFVDRRWVGRTPADRERILLRFADLVEQHGEELAQLETLEQGKSIAISRAFEVGCTLNWMRYTAGLTTKISGRTLDVSIPFPQGARYQAWTKKEPVGVVAGIVPWNFPLMIGMWKVMPALAAGCSIVIKPSETTPLTLLRVAELATQAGIPDGVFNVVTGSGAGCGAALTAHPQVAKVSFTGSTATGKQIARVAADRLTRVTLELGGKNPAIVLKDADPQWVIEGLMTGSFLNQGQVCAASSRIYIEAPLFDTLVSGFEQAVKSLQVGPGMQETAQINPVVSRAHCDKVAAYLEEARQQKAELISGSAGPDAGGYYIPPTLVVNPDAGLRLSREEVFGPVVNLVRVADGEEALRLANDSDFGLTASVWTRDLTQALNYTDRLQAGTVWVNSHTLIDANLPFGGMKQSGTGRDFGPDWLDGWCETKSVCVRY

SEQ ID NO: 26 - protein, 499 aa, Klebsiella sp. 1_1_55
MSTSQIALLASVQQFLDRQHGLYIDGAPCAAQSENRLTVWDPATGQAIATTADASPADVDRAVMSAWRAFVDRRWAGRTPADRERILLRFADLVEQHGEELAQLETLEQGKSIAISRAFEVGCTLNWMRYTAGLTTKISGRTLDVSIPFPQGARYQAWTKKEPVGVVAGIVPWNFPLMIGMWKVMPALAAGCSIVIKPSETTPLTLLRVAELATLAGIPDGVFNVVTGSGAGCGAALTAHPQVAKVSFTGSTATGKQIARVAADRLTRVTLELGGKNPAIVLKDADPQWVIEGLMTGSFLNQGQVCAASSRIYIEAPLFDTLVSGFEQAVKSLQVGPGMQETAQINPVVSRAHCDKVAAYLEEARQQKAELISGSAGPDTGGYYIPPTLVVNPDAGLRLTREEVFGPVVNLVRVADGEEALRLANDSDFGLTASVWTRDLTQALNYTDRLQAGTVWVNSHTLIDANLPFGGMKQSGTGRDFGPDWLDGWCETKSVCVRY

SEQ ID NO: 27 - protein, 499 aa, Serratia odorifera
MPDNTVTILDSVSQFLDRQHGLYIDGQSQPSQAEQRLPVHNPADGQQISTTADASAEDVARAVTSAHRAFTAGVWAQRLPAERERILLRYADLVEQHAEQLAQLETLEQGKSIMISRDFEVGCTLNWMRYTAGLATKITGQTLDVSIPMPPGARYQVYTRKEPIGVVAGIVPWNFPLMIGIWKVMPALAAGCSIVIKPSETTPLTLLRMAELASEAGIPPGVFNVVTGRGAVCGKALTEHPLVAKVSFTGSTPVGKGIARAAADRLTRVTLELGGKNPAIVLQDADITQVVEGLMMGSFLNQGQVCAASSRIYIEAPIYDNVVAGFEQAVKSLSVGPGMDTRAQITPLVSRSHRDRVAAFLDDAKAKHAELIAGANGPVGDGFYIPPTLVINPAANLNLTREEVFGPVVNLIRVADGEEALRLANDSEYGLTASLWTRSLQAAMAYTPRIQAGTVWVNTHTLIDANMPFGGFKQSGSGRDFGPDWLDAYTESKSVCIRY

SEQ ID NO: 28 - protein, 499 aa, Serratia odorifera
MPDNTQAVMDSVSRFLDRQHGLYIDGQWCESSADDRLAVYNPADGQQISSTADANAQDVARAVQSAHKAFTTGAWAQRLPAERERILLRYADLVEQHAEELAQLETLEQGKSINIARAFEVGCTLNWMRYTAGLTTKIVGQTLDVSIPMPPGAKYQVYTRKEPIGVVAGIVPWNFPLMIGMWKVMPALAAGCSIVIKPSETTPLTLLRMAELATEAGVPPGVFNVVTGRGTGCGKALTEHPLIAKVSFTGSTPVGKSIARSAADRLTRVTLELGGKNPAIVLKDADPQQVIEGLMMGSFLNQGQVCAASSRIYIEAPIYDRLVAGFEQAVKSLSVGPGMDATAQINPLVSRDHRNKVAAYLDDARAKHAELISGAAGPDAQGFYIPPTLVINPDDKLNLTREEVFGPVVNLIRVADAEEALSKANDTDYGLTASLWTTSLQQAMALTPRIQAGTVWVNTHTLIDANMPFGGFKQSGSGRDFGPDWLDAYTETKSVCIRY

SEQ ID NO: 29 - protein, 499 aa, Serratia proteamaculans
MPDNTLAVMDSVSRFLDRHHGLYIDGQWCESSAEHRLAVFNPADGKQISSTADANAQDVARAVQSAHQAFTSGVWAQRLPAERERILLRYADLLEQHTEELAQLETLEQGKSINIARMFEVGCTLNWMRYTAGLTTKITGQTLDVSIPMPPGAKYQVYTRKEPIGVVAGIVPWNFPLMIGMWKVMPALAAGCSIVIKPSETTPLTLLRMAELASEAGIPPGVFNVVTGKGTGCGKALTEHPLIAKVSFTGSTPVGKGIARAAADRLTRVTLELGGKNPAIVLKDADQQQVIEGLMAGSFLNQGQVCAASSRIYIEAPIYDSLVAGFEQAVKSLTVGPGMDANAQINPLVSSDHRNKVAAYLDDARSKHAELISGAAGPDSQGFYIPPTLVINPDEQLNLAREEVFGPVVNLIRVADAEEALSKANDTDYGLTASLWTTSLQAAMAYTPRIQAGTVWVNTHTLIDANMPFGGFKQSGSGRDFGPDWLDAYTESKSVCIRY

SEQ ID NO: 30 - protein, 495 aa, Acetobacter pasteurianus
MDSLLASVANFVSISHELYINGGAVPSSGDARLPIYDPSTGMQIASTVDATAQDVDRAVTSAFNSFKSGIWKDMRPAERERILLRLADLVERDAEILAQLETLEQGKSLAISRALEAGGAQTWIRYVAGLATTITGKTFDVSIPFPPDARYTSYTRRTPVGVVAGIIPWNFPLLIGVWKVLPALAAGCSVVAKPAETTPLTLLYLARLATEAGVPDGVFNVVTGRGTVAGSQLVNHPLVSKISFTGSTPVGKAIARSCADSLKRFSLELGGKNPAIVLDDADLEQTVQGLMLASFLNQGQVCAACSRIYVTDKMFEPLRNALTQAIQNMTVGAGMNLQAQINPVVSAAQQKKILSYVQNADTEAEQVIIGQNGPNAEGYYVPPTLIINPSPEAACVTEEIFGPVLTLTRTSDGNEALQLANTSSFGLAASVWTQNLQAAMTLPAQLEAGTVWVNSHVMIDPNMPFGGLKQSGSGADFGSDWLDSFTIQKSICIRH

SEQ ID NO: 31 - protein, 498 aa, Sphingobium chlorophenolicum
MTYTGPFTLDPTAAAFLGRAPALFIDGRSVAADGRGCLPVYDPSSGTIIAEVADASAPDVDRAVCSAHAAFVDGRWRNLRPADRERVLLRLADLLEVRAEAFAQLESLEQGKSINIARMIEVGASIDWIRYAAGLATKISGRTFDLSLPGGPTHWTAYTRREPVGVVAAIAPWNFPLMIALWKVLPALASGCSIVLKPSEVTPLTALLLAEMALEAGVPAGVFNVVTGSGAVAGRALAEHPLVAKISFTGSTATGKAIGHAAIDGMKRFTLELGGKNPALILRDAKLEKVVPGLMAGGFLNGGQVCAAVSRIYVEAPLYDDLVAALSGAIAAVTVGPGLDPEAQLNPLVSATHSAKVKSYLDDADAAGAKIVRGAAVPEEGYYVSPALILNAPAEAKLVREEVFGPVLNISRVADAEEGLRLANDNDLGLAASLWTQDIDQAMALTRRIEAGTIWVNSHVFIDPNMPFGGFKQSGLGRDFGMDWLDGYTEEKSICIAH

SEQ ID NO: 32 - protein, 499 aa, Lutiella nitroferrum
MDQNLVPVLPVVSAFLRKEHGLLVDGTSVQARAGGRIEVRNPATGEVIASVADGDEQDVEALVQSAHRAFAGGVWSGLRPADRERILLKFAEVIEAHGEELAQLETLNQGKSIHISRAIEVGASVEYVRYMAGWATKITGETMDVSIAVPQGTRYTAYTRREPAGVVAAIVPWNFPLMIAIWKLIPALAAGCTIVLKPSTETPLTALRLGELALEAGIPPGVVNVLTGRGSRAGQALASHPLVSKISFTGSTDIGKTVAHAAVDNMTRFSLELGGKNPMVVLADADVDKAIQGVLMGGFLNQGQVCAAASRLYIHRSKFDQIVEGVADTVRGMTLGSGLDLTAQVNPLVSSKQQQSVCRYLDIARAEGARVLAGGGKSDRPGYFVQPTVLTNVDHSKTVVREEIFGPVLVAMPFDSVEEAIQLANDTPYGLAASLWTNDLSAVMNLTPSIQAGTVWVNSHVPLDPNLPFGGHKQSGVGREFGRTAVESFTELKSVCIAH

SEQ ID NO: 33 - protein, 500 aa, Burkholderia ubonensis
MTSSTFVAVSDTVRRFVARDFGLFIDGDMQPAHASARLDVHDPATGERLATVADADEHDVERAVASARRAFDARVWSGLRPADRERILLKLADLIERDAETLAQLETLNQGKSIHVARAVEVGASVEYVRYMAGWATKITGQTLDVSIPFPPGARYTAYTRKEPVGVVAAIVPWNFPLMIAVWKLVPALAAGCTIVLKPSPETPLTALRLAELAREAGVPPGVFNVVTGGRVCGAALASHPSIAKISFTGSTATGKLVGAAAVQNMTRFSLELGGKNPIVMLDDVDVTQALDGVAAGAFFNQGQVCAAASRIYVHRSKFAQLADGLAGIAQSMKLGPGLDTAAQVNPLVSAHHRDKVVQHIEQARGDGHTFLAGGTPADDLPGYFVKPAVIADPRPDSAIVRDEVFGPVVVVLPFDDAADAVRLANASPYGLAASIWSNDLTRVMNLVPQIEAGTVWVNCHIPLDPSMPFGGYKQSGIGREFGQHAIDGFTETKSVCIAH

SEQ ID NO: 34 - protein, 500 aa, Burkholderia sp. 383
MSTTNFVAVSDTVRTFVARDFGLFIDGEMQPAHAAARLDVYDPATGERLATVADADERDVDRAVASAKHAFDTRVWSGLRPADRERILLKLADLIERDAETLAQLETLNQGKSIHVSRAIEVGASVEYVRYMAGWATKITGQTLDVSIPFPPGARYTAYTRKEPVGVVAAIVPWNFPLMIAVWKLIPALAAGCTIVLKPSPETPLTALRLAELALEAGVPPGVFNVVTGGRVCGAALASHPSIAKISFTGSTATGKLVGAAAVQNMTRFSLELGGKNPIVMLDDIDVAQALDGVAAGAFFNQGQVCAAASRIYVHRSKFAQLADGLAGVAQSMKLGAGLDTTAQINPLVSAHHRDKVVQHIEGARRAGLTFLAGGTPADDLPGYYVKPAVIADPHPDSAIVRDEVFGPVIVVVPFDDAADAVRLANASPYGLAASIWSNDLKRVMNLVPQIEAGTVWVNCHIPLDPSMPFGGYKQSGIGREFGQYAIEGFTETKSVCIAH

SEQ ID NO: 35 - protein, 507 aa, Burkholderia ambifaria
MTIIETPMSTTNFVAVSDTVRTFVARDFGLFIDGAMQPAHSAARLDVYDPATGERLATVADADAHDVDRAVASAKHAFDTRVWSGLRPADRERILLKLADLIEADAETLAQLETLNQGKSIHVSRAIEVGASVEYVRYMAGWATKITGQTLDVSIPFPPGARYTAYTRKEPVGVVAAIVPWNFPLMIAVWKLVPALAAGCTIVLKPSPETPLTALRLAELAREAGVPPGVFNVVTGGRTCGAALSSHPSIAKISFTGSTATGKLVGAAAVQNMTRFSLELGGKNPIVMLDDVDVAQALDGVAAGAFFNQGQVCAAASRIYVHRSKFAQLADGLAGVAQSMKLGAGLDTTAQINPLVSAHHRDKVLQHIEGARRAGLTFLAGGTPADDLPGYYVKPAVIADPHPDSAIVRDEVFGPVIVVVPFDDAADAVRLANASPYGLAASIWSNDLKRVMNLVPRIEAGTVWVNCHIPLDPSMPFGGYKQSGIGREFGQYAIEGFTETKSVCIAH

SEQ ID NO: 36 - protein, 500 aa, Burkholderia ambifaria
MSTTNFVAVSDTVRTFVARDFGLFIEGAMQPAHSAARLDVYDPATGERLATVADADAHDVDRAVASAKHAFDTRVWSGLRPAERERILLRLADLIEADAETLAQLETLNQGKSIHVSRAIEVGASVEYVRYMAGWATKITGQTLDVSIPFPPGARYTAYTRKEPVGVVAAIVPWNFPLMIAVWKLIPALAAGCTIVLKPSPETPLTALRLAELARDAGVPPGAFNVVTGGRTCGAALSSHPSIAKISFTGSTATGKLVGAAAVQNMTRFSLELGGKNPIVMLDDVDVAQALDGVAAGAFFNQGQVCAAASRIYVHRSKFAQLADGLAGIAQSMKLGAGLDTTAQINPLVSAHHRDKVVQHIEGARRAGLTFLAGGTPADDLPGYYVKPAVIADPHPDSAIVRDEVFGPVIVVVPFDDAADAVRLANASPYGLAASIWSNDLKRVMNLVPRIEAGTVWVNCHIPLDPSMPFGGYKQSGIGREFGQYAVEGFTETKSVCIAH

SEQ ID NO: 37 - protein, 500 aa, Burkholderia cenocepacia
MSNTSFVAVSDTVRTFVARDFGLFIDGAMQPAHAAARLDVYDPATGERLATVADADAHDVDRAVASARHAFDARVWCGLRPADRERILLKLADLIEADAETLAQLETLNQGKSIHVSRAIEVGASVEYVRYMAGWATKITGQTLDVSIPFPPGARYTAYTRKEPVGVVAAIVPWNFPLMIAVWKLVPALAAGCTIVLKPSPETPLTALRLAELAREAGVPPGVFNVVTGGRVCGAALASHPSIAKISFTGSTATGKLVGAAAVQNMTRFSLELGGKNPIVMLDDVDVAQALDGVAAGAFFNQGQVCAAASRIYVHRSKFAQLADGLAGVARSMKLGAGLDTTAQINPLVSAHHRDKVVQHIEGARRAGLTFLAGGTPADDLPGYYVKPAVIADPHPDSAIVRDEVFGPVIVVVPFDDAADAVRLANASPYGLAASVWSNDLKRVMNLVPQIEAGTVWVNCHIPLDPSMPFGGYKQSGIGREFGQYAIEGFTETKSVCIAH

SEQ ID NO: 38 - protein, 500 aa, Burkholderia ambifaria
MSTTNFVAVSDTVRTFVARDFGLFIDGAMQPAHSAARLDVYDPATGERLATVADADAHDVDRAVASAKHAFDTRVWSGLRPAERERILLKLAELIEADAETLAQLETLNQGKSIHVSRAIEVGASVEYVRYMAGWATKITGQTLDVSIPFPPGARYTAYTRKEPVGVVAAIVPWNFPLMIAVWKLIPALAAGCTIVLKPSPETPLTALRLAELAREAGVPPGAFNVVTGGRACGAALSSHPSIAKISFTGSTATGKLVGAAAVQNMTRFSLELGGKNPIVMLDDVDVAQALDGVAAGAFFNQGQVCAAASRLYVHRSKFAQLADGLAGVAQSMKLGAGLDTTAQINPLVSAHHRDKVVQHIEGARRAGLTFLAGGTPADDLPGYYVKPAVIADPHPDSAIVRDEVFGPVIVVVPFDDAADAVRLANASPYGLAASIWSNDLKRVMNLVPRIEAGTVWVNCHIPLDPSMPFGGYKQSGIGREFGQYAVEGFTETKSVCIAH

SEQ ID NO: 39 - protein, 521 aa, Burkholderia cenocepacia
MVRRPRASPARGPATTINETPMSNTNFVAVSDTVRTFVARDFGLFIDGAMQPSHAAARVDVYDPATGERLATVADADAHDVDRAVASAKHAFDTRVWSGLRPADRERILLKLADLIEADAETLAQLETLNQGKSIHVSRAVEVGASVEYVRYMAGWATKITGQTLDVSIPFPPGARYTAYTRKEPVGVVAAIVPWNFPLMIAVWKLIPALAAGCTIVLKPSPETPLTALRLAELAREAGVPPGVFNVVTGGRVCGAALASHPSIAKISFTGSTATGKLVGAAAVQNMTRFSLELGGKNPIVMLDDVDVAQALDGVAAGAFFNQGQVCAAASRIYVHRSKFAQLADGLAGVARSMKLGAGLDTTAQINPLVSAQHRDKVVQHIEGARRAGLTFLAGGTPADDLPGYYVKPAVIADPHPDSAIVRDEVFGPVIVVVPFDDAADAVRLANASPYGLAASIWSNDLKRVMNLVPQIEAGTVWVNCHIPLDPSMPFGGYKQSGIGREFGQYAIEGFTETKSVCIAH

SEQ ID NO: 40 - protein, 500 aa, Burkholderia ambifaria
MSTTNFVAVSDTVRTFVARDFGLFIDGAMQPAHSAARFDVYDPATGERLATVADADAHDVDRAVASAKHAFDTRVWSGLRPADRERILLKLADLIERDAETLAQLETLNQGKSIHVSRAIEVGASVEYVRYMAGWATKITGQTLDVSIPFPPGARYTAYTRKEPVGVVAAIVPWNFPLMIAVWKLIPALAAGCTIVLKPSPETPLTALRLAELAREAGVPPGAFNVVTGGRTCGAALSSHPSIAKISFTGSTATGKLVGAAAVQNMTRFSLELGGKNPIVMLDDVDVAQALDGVAAGAFFNQGQVCAAASRLYVHRSKFAQLADGLAGVAQSMKLGAGLDTTAQINPLVSAHHRDKVVQHIEGARRAGLTFLAGGTPADDLPGYYVKPAVIADPHPDSAIVRDEVFGPVIVVVPFDDAADAVRLANASPYGLAASIWSNDLKRVMNLVPQIEAGTVWVNCHIPLDPSMPFGGYKQSGIGREFGQYAIEGFTETKSVCIAH

SEQ ID NO: 41 - protein, 500 aa, Burkholderia multivorans
MSTTNFVAVSDTVRTFLARDFGLFIDGAMQPAHAAARLDVYDPATGERLATVADADEYDVDRAVASAKRAFDARVWRGLRPADRERILLKLAELMERDAETLAQLETLNQGKSIHVSRAVEVGASVEYVRYMAGWATKITGQTLDVSIPFPPGARYTAYTRKEPVGVVAAIVPWNFPLMIATWKLIPALAAGCTVVLKPSPETPLTALRLAELALEAGVPPGVFNVVTGGRTCGAALARHPSIAKISFTGSTETGKLVGAAAVQNMTRFSLELGGKNPIVMLEDVDVGQALDGVAAGAFFNQGQVCAAASRIYVHRSKFARLADGLAEHARAMTLGPGLDTAAQINPLVSAQHRDKVVRHIEQARRDGVTFLAGGTRVDELPGYYVRPAVIADPRADSAIVRDEVFGPVIVVLPFDDAADAVRLANASPYGLAASIWSNDLTRVMNLVPQIEAGTVWVNCHIPLDPSMPFGGYKQSGIGREFGQYAIEGFTETKSVCIAH

SEQ ID NO: 42 - protein, 499 aa, Burkholderia sp. CCGE1001
MSSDAKVALCDAVRAFVGREHGLFIDGAACLAHSPRRSNVFDPATGNVLTTVPDADATDVDRAVTSARVAFEARVWRGLRPADRERILLRFADVLEAHAEELAQLETLNQGKSINVARAIDVGATLEYVRYMAGWATKIVGETLDVSIPFPPGARYTAFTRHEPVGVVAGIVPWNFPMMIAVWKLVPALAAGCSVVIKPSPETPLTALRLAELAVEAGIPAGAFNVVTGGAECGAALAAHPGINKISFTGSTPTGKRVGIAAVQNMTRFSLELGGKNPAVMLADIDVEQAVQGALAGGLLNQGQVCAAVSRIYVHRSKYAKVVEGLADSVSAMTMGPGLDPSAHINPLVSAHHRARVEEHLVRARAEGLRFAAGGARVEEPGYYVRPAVIADVPPEAAIVRDEVFGPVLALAPFDDVAHALRLANDSPYGLAASLWTNDLRAAMNLVPQIEAGTVWVNCHVPLDPGMPFGGYKQSGIGREFGRHAIEGFTETKSVCIAH

SEQ ID NO: 43 - protein, 500 aa, Burkholderia cenocepacia
MSNTNFVAVSDTVRTFVARDFGLFIDGAMQTAHAAARVDVYDPATGERLATVADADAHDVDRAVASAKHAFDTRVWSGLRPADRERILLKLADLIEADAETLAQLETLNQGKSIHVSRAVEVGASVEYVRYMAGWATKITGQTLDVSIPFPPGARYTAYTRKEPVGVVAAIVPWNFPLMIAVWKLIPALAAGCTIVLKPSPETPLTALRLAELAREAGVPPGVFNVVTGGRVCGAALASHPSIAKISFTGSTATGKLVGAAAVQNMTRFSLELGGKNPIVMLDDVDVAQALDGVAAGAFFNQGQVCAAASRIYVHRSKFAQLADGLAGVAQSMKLGAGLDTTAQINPLVSAQHRDKVVQHIEGARRAGLTFLAGGSPADDLPGYYVKPAVIADPHPDSAIVHDEVFGPVIVVVPFDDAADAVRLANASPYGLAASIWSNDLTRVMNLVPQIEAGTVWVNCHIPLDPSMPFGGYKQSGIGREFGQYAIEGFTETKSVCIAH

SEQ ID NO: 44 - protein, 500 aa, Burkholderia multivorans
MSTTNFVAVSDTVRTFLARDFGLFIDGAMQRAHAAARLDVYDPATGERLATVADADEHDVDRAVASAKRAFDARVWRGLRPADRERILLKLADLIERDAETLAQLETLNQGKSIHVSRAVEVGASVEYVRYMAGWATKITGQTLDVSIPFPPGARYTAYTRKEPVGVVAAIVPWNFPLMIATWKLIPALAAGCTVVLKPSPETPLTALRLAELALEAGVPPGVFNVVTGGRTCGAALARHPSIAKISFTGSTETGKLVGAAAVQNMTRFSLELGGKNPIVMLDDVDVGQALGGVAAGAFFNQGQVCAAASRIYVHRSKFAQLADGLAEHARAMTLGPGLDTTAQINPLVSAQHRDKVVRHIEQARRDGVTFLAGGTRVDELPGYYVRPAVIADPRADSAIVRDEVFGPVIVVLPFDDAADAVRLANASPYGLAASIWSNDLTRVMNLVPQIEAGTVWVNCHIPLDPSMPFGGYKQSGIGREFGQYAIEGFTETKSVCIAH

SEQ ID NO: 45 - protein, 501 aa, Burkholderia dolosa
MTSSPTFVAVSDTVRAFVARDFGLFIDGDMQRAHAAARLDVYDPATGERLATVADADAHDVDRAVASAKSAFDARVWSGLRPADRERILLKLADLIERDAETLAQLETLNQGKSIHVSRAVEVGASVEYARYMAGWATKITGQTLDVSIPFPPGTRYTAYTRKEPVGVVAAIVPWNFPLMIAVWKLIPALAAGCTIVLKPSPETPLTALRLAELAREAGVPPGAFNVVTGGRTCGAALASHPSIAKISFTGSTATGKLVGAAAVQNMTRFSLELGGKNPIVMLDDVDVAQALDGVAAGAFFNQGQVCAAASRIYVHRSRFSQLADGLAGVANAMKLGPGLDMAAQVNPLVSAHHRDKVVAHIERARRDGLTFLAGGTLADDLSGYFVKPAVIADPHPDSAIVRDEVFGPVIVVVPFDDAADAVRLANASPYGLAASIWSNDLKRVMNLVPQIEAGTVWVNCHIPLDPSMPFGGYKQSGIGREFGQYAIEGFTETKSVCIAH

SEQ ID NO: 46 - protein, 500 aa, Burkholderia oklahomensis
MTQTDFVTVTDTVRAFTEREFGIFIDGAMRAAHSPRRLDVFDPATGERLARVPDADAHDVDAAVASAKRAFDARAWSGLRPADRERILLKLADALEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGGRECGAALASHPSIRKISFTGSTATGKLVGAAAVQNMTRFSLELGGKNPIVMLEDVDVAQALEGVAAGAFFNQGQVCAAASRIYVHRSRFRRLADGLAGVAASMRLGPGLDPAAQINPLVSAHHRDKVVEHVERARRDGLTFLAGGAPADDLPGYFVRPAVIADATHDSAIVRDEVFGPVVVVLPFDDPAEAVRLANASPYGLAASLWSNDLKAVMDLVTQIEAGTVWVNCHIPLDPSMPFGGYKQSGIGREFGQYAIDGFTETKSVCIAH

SEQ ID NO: 47 - protein, 500 aa, Burkholderia pseudomallei
MTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRSVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 48 - protein, 521 aa, Burkholderia pseudomallei
MRAARAATGTSRMIHPQHETHMTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 49 - protein, 500 aa, Burkholderia pseudomallei
MTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVHKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 50 - protein, 500 aa, Burkholderia pseudomallei
MTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 51 - protein, 500 aa, Burkholderia mallei
MTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFFAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 52 - protein, 509 aa, Burkholderia mallei
MIQPQHETHMTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFFAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 53 - protein, 500 aa, Burkholderia pseudomallei
MTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARECGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 54 - protein, 509 aa, Burkholderia pseudomallei
MIHPQHETHMTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPGAHYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 55 - protein, 500 aa, Burkholderia pseudomallei
MTQTDLVAVADTVRAFTEREFGVFIDGAMRAAHSPRRLDVFDPATGARLSRVPDADAHDVDAAVASAQRAFDARAWSGLRPAERERILLKLADVLEAHAEELAQLETLNQGKSILVSRGVEVGATIEYVRYMAGWATKITGQTLDVSIPFPPDARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 56 - protein, 573 aa, Burkholderia thailandensis
MKRTRRARRRTSARSARSDAPYSPAQPGRVPAAPPNLPICANPPRSRVVTLVRPIRAAMSGAGRPIEPQHETHMTQTDLVAVADTVRAFTKREFGIFIDGAMRAAHSPRRLDVYDPATGERLSRVPDADAHDVDAAVASAKRAFDARVWSGLRPADRERVLLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGGRECGAALAGHPSVRKISFTGSTATGKLVGAAAVQNMTRFSLELGGKNPIVMLDDVDVDAALGGVAAGAFFNQGQVCAAASRIYVHRSKFRRLAQGLADVAAAMRLGPGLDPAAQINPLVSAHHRDKVVQHIEVARREGLTFLTGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 57 - protein, 500 aa, Burkholderia thailandensis
MTQTDLVAVADTVRAFTKREFGIFIDGAMRAAHSPRRLDVYDPATGERLSRVPDADAHDVDAAVASAKRAFDARVWSGLRPADRERVLLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGGRECGAALAGHPSVRKISFTGSTATGKLVGAAAVQNMTRFSLELGGKNPIVMLDDVDVDAALGGVAAGAFFNQGQVCAAASRIYVHRSKFRRLAQGLADVAAAMRLGPGLDPAAQINPLVSAHHRDKVVQHIEVARREGLTFLTGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 58 - protein, 500 aa, Burkholderia thailandensis
MTQTDLVTVADTVRAFTEREFGIFIDGAMRAAHSPRRLDVYDPATGERLSRVPDADAHDVDAAVASAKRAFDARVWSGLRPADRERVLLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGGRECGAALSGHPSVRKISFTGSTATGKLVGAAAVQNMTRFSLELGGKNPIVMLDDVDVDAALGGVAAGAFFNQGQVCAAASRIYVHRSKFRRLAQGLADVAAAMRLGPGLDPAAQINPLVSAHHRDKVVQHIEVARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 59 - protein, 492 aa, Burkholderia phytofirmans
MTEHKLAATLPHTMFINGEKTGSAAGKTFPVFNPATAEEIAQIPDASEADIDHAVRTSKAAFESDAWRRMPPAVREGLLLKLADLVERHSDELATLETLNQGKLIGFSKMLEVAGSVQWLRYMAGWATKIEGSTFDLSIPFPPGTRYNASTKRVPAGVVAAIVPWNFPLLMAVWKIAPALACGCTVVLKPAEETPLTAIRLAELAHEAGFPAGVFNVVTGRGETAGAALVRHPLVKKVTFTGSTEVGRIIGRQCADDLKRASLELGGKSPVIVLDDCDPRKAIEGAAGAIFFNHGQVCTAGSRLYVARSIYDEVVQGIAAVADGITLGSGFDAATQMGPMVSARHRDKVAGMIAQGKDEGGEIVSRDARVEREGYFVRPTVIANRACKPLAVVKEEVFGPVLVAMPYDDLDEVLAQANASEYGLGASVWTNQLDKALRLVDGIEAGTVWVNTHNMVDPAMPFGGFKASGIGREHGKSIIESYTESKSVCIAY

SEQ ID NO: 60 - protein, 432 aa, Burkholderia pseudomallei
RAFGARAWSGLRPAERERILLKLADVLEAHAEELAQLETLNQGKSILVSRGVEVGATIEYVRYMAGWATKITGQTLDVSIPFPPGARYTAYTRKEPVGVVAAIVPWNFPLMIAVWKLVPALAAGCTVVLKPSPETPLTALRLAELALEAGVPAGVFNVVTGARACGAALASHPAVRKISFTGSTATGKLVGAAAVQNMTRFSLELGGKNPIVMLEDVDVDAALGGVAAGAFFNQGQVCAAASRIYVHRSRFRRLAEGLAGVASAMRLGPGLDPAAQINPLVSAHHRDTVARHIDAARREGLTFLAGGTRADDLPGYFVRPAVIADAAHDSAIVRDEVFGPVVVVLPFDDPAEAVRLANASPYGLAASLWSNDLKAVMDLVPQIEAGTVWVNCHIPLDPSMPFGGYKQSGIGREFGQYAIEGFTETKSVCIAH

SEQ ID NO: 61 - protein, 501 aa, Burkholderia thailandensis
MNLAALSTQHQRQSGFLARRQFGNWLDGSAAEPRSGRYLPVVDPATEMTIAEVAASDARDVDAAVAAARRAFDSGDWPRMRPASREKLLHRLADRIERHADELAALETLETGKLVGVARAIDVLGGAEYVRYVAGWATKLEGSTLDTSIAAPAGTEYFAYTRREAVGVVGAIVPWNFPLAIALWKIATALACGCTVVLKPSEETPLTALRLGELAQEAGLPDGVLNVVTGTGAEAGAALVAHPGVDKITFTGSVGVGRAIGHAAVDRMARFTLELGGKSPLIVFDDADPDVAARGAAQGIFFNQGQVCTAGSRVYVQKRLFEQVVAGIAAAAESMKIGSGFDPNTQIGPLVSKRHFERVLSHVDAAKEEGATLVTGGTRALDGGYFVKPTVFVDAAPSMRIVREEVFGPVVTVTPFDTLDDAVRLANDTDFGLAASVWSQNLSRVHRVVPRLKAGIVWVNTHNMLDSNLPFGGFKQSGYGRELGRAALEQFTELKSVCIAH

SEQ ID NO: 62 - protein, 492 aa, Burkholderia sp. CCGE1002
MKIEALLANLRTQMIVGGRAVDARSGKTFAVYDPASGREIAQVPDGDAEDVAAAVAAAKSAFESNEWRRMPPAAREHLLLKLADLVEQHGDELAALETLNQGKLLGFSRMLEVGGSAQWLRYMAGWATKIEGSTVDLSLSFPPGVQYRASTQRVPAGVVAAIVPWNFPLLMAVWKIAPALACGCTVVLKPAEETPLTAIRLAELALEAGLPAGVLNVVTGRGETAGAALVRHPDVDKVTFTGSTEVGRLIGAQCGRDIRRASLELGGKSPVIVLDDCDPRKAIEGAAGAIFFNHGQVCTAGSRLYVPRSMYAQIVEGVAQVANSLVLGSGFDEKTQMGPLVSARHRDKVVGMIAEGRAQGGEIIAGGSARDGDGYFVRPTVVANEARRPLSLVNEEVFGPVLVAMPYDDLEEAISAANSSEYGLGASVWTNQLDKALRVVDRMEAGTVWVNSHNMVDPALPFGGFKSSGVGREHGRAIIDAYTETKSVCFAY

SEQ ID NO: 63 - protein, 527 aa, Achromobacter piechaudii
MHGPTSQRAMPLLFWLHTFRWSFPMSSARSSAADNAAADRPELAAIREFMLIDGKPVHEGQGQPVPVHDPATGRVIAHQPDAGPAQVDLAVQAARRAFDSGTWRDMLPAGRERLLLKLADLIEQHGVELARLETLNNGKLLGIAQGLEVGASAQWLRYMAGWATKITGDTLSLSIPFPPGTHYHAYTLAQPVGVVGAIIPWNFPLLMAVWKIAPALAAGCTVVLKPAEETPLTALRLAELVMQAGFPPGVVNVITGRGETAGAALVAHPGIDKIAFTGSTEVGKLIGRRAMDDMKRVSLELGGKSPVIVLDDCDVDCAVQGAAAAIFFNQGQVCTAGSRLYVQRGLYAKVVQGLADVASSMTLGSGFDPATQIGPLISSQHQQRVLDYIGIGRAEGGRVLTGGAAGEGDGYFVRPTVFADVPQHGRIAQEEIFGPVVVAQPFDSLDDAVRLANDSAFGLGASIWSNDLTRVQRLIPRIDAGTVWVNTHNMLDPNMPFGGFKQSGVGREHGKAVLEMYLEKKSVCMAY

SEQ ID NO: 64 - protein, 497 aa, Xenorhabdus bovienii
MSEITLLKPVTDFLQRPHGNYINGLSLPGQGNRTFSVVNPASDEMIAKVNQGEEAEINLAMEAASKAFHGSWAQTSPMERGKCLNRLADLLQKHGEELAQLESLCSGKPIQLARMLDVGASADYLRYFAGWSSKISGETLNVSLPSLKGEKYTAFTRREPIGVVVGIIPWNFSIMIAIWKLGAALACGCTLVLKPSEYTPLTMLRVAELAKEAGIPDGVINVVNGSGARVGSALLAHPHCAKVTFTGSVPTGTIIGKSAIEYGLSRATLELGGKNAAAFLSDMSVEKIVEGVLEAGYLNQGQICAAAERFYIPSVHMDAVLKLLSERLSAMKIGSPLDESTEMGPLANKEHYEKILSLFDKARQDGSEIVYGGHALEGAGFFVTPTIIRAKSAEDTLMQEETFGPIGTFLSYDDEEELIVMMNATPFGLSASLWTNDLSKAMRMIPRIQVGTLWINMHTFLDPALPFGGVKSSGIGREFGSAFIEHYTELKSVMVRY

SEQ ID NO: 65 - protein, 497 aa, Providencia alcalifaciens
MSELTLLPEVREFLKRQHGHFINGLPVSGKGEAYFDVVNPATEQVIAKVKEGTREEVDIAMNVAYAAFKGSWANTTPMERGNCLNRLADLLEKHLEELAQLETLSSGKTIQLSRFLEVGSAAQFLRYFAGWATKISGETLNVSLPSFNGEKYSAFTQREPVGVVAGIIPWNFSIMISIWKLAAALTCGCTIVLKPSEFTPLTMLRVAELAKEAGIPDGVINIVNGGGREVGPALISHELCSKVTFTGSVPTGLAVGRSAMEGKLTRVTLELGGKNGAAFLADLSVDKIVSGIIEAGYLNQGQICAAAERFYVPSTLMDAVLEELKARLSAMKIGSPLDEDTQMGPLANKAHYNKILSLFEKARQDGSEIIYGGQPIAGAGYFVPPTIIRAKSPDDVLMKEETFGPIGTFLAYDDEEELIKMMNSTPFGLAASVWTNDLSKAMRMVSQIEAGTVWVNMHTFLDPAVPFGGIKSSGIGREFGSAFIEHYTELKSVMVRY

SEQ ID NO: 66 - protein, 501 aa, Burkholderia thailandensis
MNLAALSTQHQRQSGFLARRQFGNWIDGAAAEPRSGRYLPVVDPATEMTIAEVAASDARDVDAAVAAARRAFDSGDWPRMRPASREKLLHQLADRIERYADELAALETLETGKLVGLARAIDVLGGAEYVRYMAGWATKLEGSTLDTSIAVPAGTEYFAYTRREAVGVVGAIVPWNFPLAIALWKVATALACGCTVVLKPSEETPLTALRLGELAQEAGLPDGVLNVVTGTGADTGAALVAHPGVDKITFTGSVGVGKAIGHAAVDRMARFTLELGGKSPLIVFDDADPDVAARGAAQGIFFNQGQVCTAGSRVYVHKSLFERVVAGIAAAAESMKIGSGFDPNTQIGPLVSKRHFERVLGHIDAAKEEGATLVTGGTRALDGGYFVKPTVFVDAAPSMRIVREEVFGPVVTVTPFDTVDDAVRLANDSDFGLAASVWSQNLSRVHRVVPRLKAGIVWVNTHNMLDNNLPFGGVKQSGYGRELGRAALEQFTELKSVCIAH

SEQ ID NO: 67 - protein, 501 aa, Burkholderia thailandensis
MNLAALSTQHQRQSGFLARRQFGNWIDGAAAEPRSGRYLPVVDPATEMTIAEVAASDARDVDAAVAAARRAFDSGDWPRMRPASREKLLHRLADRIERYADELAALETLETGKLVGVARAIDVLGGAEYVRYMAGWATKLEGSTLDTSIAVPADTEYFAYTRREAVGVVGAIVPWNFPLAIALWKVATALACGCTVVLKPSEETPLTALRLGELAQEAGLPDGVLNVVTGTGADTGAALVAHPGVDKITFTGSVGVGKAIGHAAVDRMARFTLELGGKSPLIVFDDADPDVAARGAAQGIFFNQGQVCTAGSRVYVHKSLFERVVAGIAAAAESMKIGSGFDPNTQIGPLVSKRHFERVLGHIDAAKEEGATLVTGGTRALDGGYFVKPTVFVDAAPSMRIVREEVFGPVVTVTPFDTVDDAVRLANDSDFGLAASVWSQNLSRVHRVVPRLKAGIVWVNTHNMLDNNLPFGGVKQSGYGRELGRAALEQFTELKSVCIAH

SEQ ID NO: 68 - protein, 503 aa, Achromobacter xylosoxidans
MSSASAIAAGHAGSDRPELAAIREAMLIDGKPVRVGQGAPIAVHDPATGEVIAHQPDAGPLQVDLAVQAARRAFESGPWRDMLPAGRERLLLKLADLVELHGTELARLETLNNGKLLGVAQGLEVGSGAQWLRYMAGWTTKITGDTLSLSIPFPPGVRYSAYTLPQAVGVVAAIIPWNFPLLMAIWKIAPALAAGCTVVLKPAEETPLTALRLAELVLEAGFPPGVVNVVTGRGETAGAALVAHPGVDKIAFTGSTEVGKLIGRAAMDDMKRVSLELGGKSPVIVLDDCDVDRAVQGAAAAIFFNQGQVCTAGSRLYVQRNLYPKVVEGLADLAAGMRLGSGFDPATQVGPLVSARHQKRVMDYIDIGRNEGGRVLAGGGRGTGSGYFVQPTVFADVPSDARIAREEIFGPVVVAQPFDTLDDAVRLANDSAYGLGASLWSNDLSRVQSLIPRVDAGTVWVNTHNMLDPNMPFGGFKQSGIGREHGRAVLEMYLERKSVCIAY

SEQ ID NO: 69 - protein, 497 aa, Providencia rustigianii
MSELALLPEVTEFLKRQHGHFINGLPVSGKGNTYFDVVNPATEQVIAKVKEGTLAEVDAAMDAAYTAFKGSWANTTPMERGNCLSRLADLLEKHLEELAQLETLSSGKTIQLSRFLEVGSAAQFLRYFAGWATKISGETLNVSLPSFHGEKYSAFTQREPVGVVAGIIPWNFSIMISIWKLAAALTCGCTIVLKPSEFTPLTMLRVAELAKEAGIPDGVINIVNGGGREVGPALIHHSLCSKVTFTGSVPTGLAVGRSAMESKLTRVTLELGGKNGAAFLADLPVDKIVNGIIEAGYLNQGQICAAAERFYIPSKLMDDVLDALKVRLSTMKIGSPLDETTEMGPLANKAHYDKILSLFEKARQDGSEIIYGGQPIAGAGYFVPPTIIRANSPNDILMQEETFGPVGTFLSYDDEDELISMMNSTPFGLAASVWTNDLGKAMRMVSQIEAGTVWVNMHTFLDPAVPFGGIKSSGIGREFGSAFIEHYTELKSVMIRY

SEQ ID NO: 70 - protein, 501 aa, Burkholderia oklahomensis
MNLADLSTQHQRQSGFLARRAFGNWIDGRAAEPRSGRYLPVVDPATEMTIAEVAASDARDVDAAVAAARRAFDSGDWPRMRPANREKLLYQLAELIERHADELAALETLETGKLVGIARAIDVLGGAEYVRYMAGWATKIEGSTLDTSIAVPADTEYFAYTRREAVGVVGAIVPWNFPLAIALWKVATALACGCTVVLKPSEETPLTALRLGELAQQAGLPDGVLNIVTGTGGEAGAALVAHPGVDKITFTGSVGVGKAIGHAAVDRMARFTLELGGKSPLIIFDDADPDVAARGAAQGIFFNQGQVCTAGSRVYVQKRHFERVVAGIAAAAESMKIGSGFDPRTQIGPLVSKRHFERVLGHIGAAKTEGATLVTGGARAFDGGYFVKPTVFVDTTPSMRIVREEVFGPVVTVTPFDTVDDAVRLANDSDFGLAASVWSQNLSLVHRVVPRLKAGIVWVNTHNMLDNNLPFGGFKQSGYGRELGRAALEQFTELKSVCIAH

SEQ ID NO: 71 - protein, 501 aa, Burkholderia oklahomensis
MNLADLSTQHQRQSGFLARRAFGNWIDGRAAEPRSGRYFPVVDPATEMTIAEVAASDARDVDAAVAAARRAFDSGGWPRMRPANREKLLYQLAELIERHADELAALETLETGKLVGIARAIDVLGGAEYVRYMAGWATKIEGSTLDTSIAVPADTEYFAYTRREAVGVVGAIVPWNFPLAIALWKVATALACGCTVVLKPSEETPLTALRLGELARQAGLPDGVLNIVTGTGGEAGAALVAHPGVDKITFTGSVGVGKAIGHAAVDRMARFTLELGGKSPLIIFDDADPDVAARGAAQGIFFNQGQVCTAGSRVYVQKRHFERVVAGIAAAAESMKIGSGFDPRTQIGPLVSKRHFERVLGHIGAAKAEGATLVTGGARAFDGGYFVKPTVFVDTTPSMRIVREEVFGPVVTVTPFDTVDDAVRLANDSDFGLAASVWSQNLSLVHRVVPRLKAGIVWVNTHNMLDNNLPFGGFKQSGYGRELGRAALEQFTELKSVCIAH

SEQ ID NO: 72 - protein, 497 aa, Providencia rettgeri
MSELTLLPEVSEFLKRQHGHFINGLSVSGKGDTFFDVVNPATEQVIAKVKEGTLAEVDAAMDAAHTAFKGVWANTTPMERGNCLNRLADLLEKHLEELAQLETLCSGKTIQLSRFLEVGSSAQFLRYFAGWATKISGETLNVSLPSFNGEKYSAFTQREPVGVVAGIIPWNFSIMISIWKLAAALTCGCTIVLKPSEFTPLTMLRVVELAKEAGVPDGVINIVNGGGREVGPALIHHPLCSKVTFTGSVPTGLAVGRSAMEGKLTRVTLELGGKNGAAFLADLPVEKIVNGIIEAGYLNQGQICAAAERFYIPSKLMDEVLAELKTRLSAMKVGSPLDETTEMGPLANKAHYEKILGLFEKARQDGSEIIYGGQPIAGAGYFVPPTIIRANSPNDVLMKEETFGPIGTFLSYDDEEELIEMMNSTPFGLAASLWTNDLSKAMRMISRIEAGTVWVNMHTFLDPAVPFGGVKSSGIGREFGSAFIEYYTELKSVMVRY

SEQ ID NO: 73 - protein, 497 aa, Photorhabdus asymbiotica
MSDITLLQQVTAFLQRNHGHYINGQSVHGQENQTFSVVNPAVDEVIATVNQGGETEVNAAMQAAHTAFHGVWAQTSPMERGHCLNRLADLLLAHREELAQLESLCSGKTIQLSRMLEIDSSAQFLRYFAGWSSKISGETLNVSLPSFKGEQYTAFTRREPIGVVVGIIPWNFSIMVAIWKMAAALTCGCTIVLKPSEYTPLTMLRVAELAKEAGIPDGVINVINGSGSVLGPALIGHPLCAKVTFTGSVPTGITVGKSAMEQGLTRATLELGGKNGAAFLADMPVEKIVDGVLEAGYLNQGQICAAAERFYIPASHMDEVLKLLSERLAAMKMGSPLDESTEMGPLANKEHYNKILSLFEQARQEGSEIVYGGHALTGPGFFVAPTVIRANSAEDSLMKEETFGPVGTFFSYNDEEELIELMNSTPFGLAASLWTNDLSKAMRMIPRIEAGTVWVNMHTFLDPALPFGGVKSSGIGREFGSAFIEHYTELKSVMVRY

SEQ ID NO: 74 - protein, 495 aa, Pseudomonas aeruginosa
MSIAIDPSVSAFLRSPHGLLIDGESGPARSGADMPLYDPATGAELARVARAGAEDVDRAVAAARRAFEGSWAGQRPADRERLLLRLAERVEAHGEQLAQLETLNNGKSINLSRALEVGASVEFIRYMAGWATKIEGRSLDLSIAAVPGARYRAYTVPEPVGVVGAIVPWNFPLLMAIWKIVPALACGCTVVLKPADETPLTALRLGQLCLEAGIPPGVVNIVTGTGAEAGAALAAHPGIDKLAFTGSTPVGKLIGHAAVENMTRFSLELGGKSPVIILDDTSLDMAAAGSAGAIFFNQGQVCTAGSRLYVQRKRFDQVLERLAAIAGDLSIGPGLDPTTQINPLVSARQQERVLGMIESGVAEGASVVCGGARQGETGFYVQPTILADVTPGMQVVREEIFGPVLVATPFDDLDEAVRLANDSIYGLGASIWSNDLRQVMDLVPRIKAGTVWVNAHNLLDPSMPFGGFKQSGIGREMGHAAIEAYTENKSVCIAY

SEQ ID NO: 75 - protein, 495 aa, Pseudomonas aeruginosa
MSIAIDPSVSAFLRSPHGLLIDGESGPARSGADMPLYDPATGTELARVARAGAEDVDRAVAAARRAFEGNWAGQRPADRERLLLRLAERIEAHGEQLAQLETLNNGKSINLSRALEVGASVEFIRYMAGWATKIEGRSLDLSIAAVPGARYRAYTVPEPVGVVGAIVPWNFPLLMAIWKIVPALACGCTVVLKPADETPLTALRLGQLCLEAGIPPGVVNIVTGTGAEAGAALAAHPGIDKLAFTGSTPVGKLIGHAAVENMTRFSLELGGKSPVIILDDTSLDMAAAGSAGAIFFNQGQVCTAGSRLYVQRKRFDQVLERLAAIAGDLSIGPGLDPTTQINPLVSARQQERVLGMIESGVAEGASVVCGGARQGETGFYVQPTILADVTPGMQVVREEIFGPVLVATPFDDLDEAVRLANDSIYGLGASIWSNDLRQVMDLVPRIKAGTVWVNAHNLLDPSMPFGGFKQSGIGREMGHAAIEAYTENKSVCIAY

SEQ ID NO: 76 - protein, 495 aa, Pseudomonas aeruginosa
MSIAIDPSVSAFLRSPHGLLIDGESGPARSGADMPLYDPATGAELARVARAGAEDVDRAVAAARRAFEGNWAGQRPADRERLLLRLAERVEAHGEQLAQLETLNNGKSINLSRALEVGASVEFIRYMAGWATKIEGRSLDLSIAAVPGARYRAYTVPEPVGVVGAIVPWNFPLLMAIWKIVPALACGCTVVLKPADETPLTALRLGQLCLEAGIPPGVVNIVTGTGAEAGAALAAHPGIDKLAFTGSTPVGKLIGHAAVENMTRFSLELGGKSPVIILDDTSLDMAAAGSAGAIFFNQGQVCTAGSRLYVQRKRFDQVLERLVAIAGDLSIGPGLDPTTQINPLVSARQQERVLGMIESGVAEGASVVCGGARQGETGFYVQPTILADVTPGMQVVREEIFGPVLVATPFDDLDEAVRLANDSIYGLGASIWSNDLRQVMDLVPRIKAGTVWVNAHNLLDPSMPFGGFKQSGIGREMGHAAIEAYTENKSVCIAY

SEQ ID NO: 77 - protein, 497 aa, Photorhabdus luminescens
MSDINLLQPVMAFLQHNHGHYINGQPVSGQGSETFSVINPATDEIIATVNQGGKAEVNAAMQAAQAAFHGVWAQTSPMERGHCLNRLADLLLAHREELAQLETLCSGKTIQLSRMLEIDSSAQFLRYFAGWSSKISGETLNVSLPSFKGEQYTAFTRREPIGVVVGIIPWNFSIMIAIWKMAAALTCGCTIVLKPSEYTPLTMLRVAELAKQAGIPDGVINVINGSGSVLGPALIGHPLCAKVTFTGSVPTGIAVGKSAMEQGLTRATLELGGKNGAAFLADMSVEKIVDGILEAGYLNQGQICAAAERFYIPASHMDDVLKLLSERLAAMKIGSPLDDSTEMGPLANKAHYDKILSLFEQARQEGSEIVYGGHALAGPGFFVAPTVIRANSPEDSLMKEETFGPVGTFLSYNDEEELIGLMNSTPFGLAASLWTNDLSKAMRMIPRIEAGTVWVNMHTFLDPALPFGGTKSSGIGREFGSAFIEHYTELKSVMVRY

SEQ ID NO: 78 - protein, 495 aa, Pseudomonas aeruginosa
MSIAIDPSVSAFLRSPHGLLIDGESGPARSGADMPLYDPATGAELARVARAGAEDVDRAVAAARRAFEGNWAGQRPADRERLLLRLAERIEAHGEQLAQLETLNNGKSINLSRALEVGASVEFIRYMAGWATKIEGRSLDLSIAAVPGARYRAYTVPEPVGVVGAIVPWNFPLLMAIWKIVPALACGCTVVLKPADETPLTALRLGQLCLEAGIPPGVVNIVTGTGAEAGAALAAHPGIDKLAFTGSTPVGKLIGHAAVENMTRFSLELGGKSPVIILDDTSLDMAAAGSAGAIFFNQGQVCTAGSRLYVQRKRFDQVLERLAAIAGDLSIGPGLDPTTQINPLVSARQQERVLGMIESGVAEGASVVCGGARQGETGFYVQPTILADVTPGMQVVREEIFGPVLVATPFDDLDEAVRLANDSIYGLGASIWSNDLRQVMDLVPRIKAGTVWVNAHNLLDPSMPFGGFKQSGIGREMGHAAIEAYTENKSVCIAY

SEQ ID NO: 79 - protein, 497 aa, Burkholderia xenovorans
MADIRILSEVETFLARQHRQYIDGQAVAAMDTVATDIINPSNRKVVASVRQATPAQVQHAVASAHEAFTGVWQQTSAARRGELLNRLADLMQAHREELAQIESLSSGKLIGLSRAFEIDYSIAFLRYYAGWATKIHGQTMNLSLPSSNGEQYTGFTLRQPIGVVAGIVPWNFSIMIAVWKFGSALACGCTAVIKPSEFTPLTMLRVAELALEAGIPPGVLNIVNGNGRTVGTALIAHPKVAKVTFTGSVPTGVGVGVAAMQAGLKHVTLELGGKNPAGFLRDFPVERTVDGIIEAAYLHQGEVCASAERFYVHRSRIDDVLEALHHTLATLKIGSALDESAQFGPLANAAHFAKVMAFFEKARAQDGEIVHGGTVAPGDGFFVQPTAIPARSQADTIMTEETFGPVASFLAYDDEEEMLHYMNDTHFGLTASIWTNDLSKALRFVPRVEAGTVWVNMHNYIDPAMPFGGVKSSGIGREFGEAFIEYFTELKSVIVRY

SEQ ID NO: 80 - protein, 495 aa, Pseudomonas aeruginosa
MSIAIDPSVSAFLRSPHGLLIEGESGPARSGADMPLYDPATGAELARVARAGAEDVDRAVAAARRAFEGNWAGQRPADRERLLLRLAERIEAHGEQLAQLETLNNGKSINLSRALEVGASVEFIRYMAGWATKIEGRSLDLSIAAVPGARYRAYTVPEPVGVVGAIVPWNFPLLMAIWKIVPALACGCTVVLKPADETPLTALRLGQLCLEAGIPPGVVNIVTGTGAEAGAALAAHPGIDKLAFTGSTPVGKLIGHAAVENMTRFSLELGGKSPVIILDDTSLDMAAAGSAGAIFFNQGQVCTAGSRLYVQRKRFDQVLERLAAIAGDLSIGPGLDPTTQINPLVSARQQERVLGMIESGVAEGASVVCGGARQGETGFYVQPTILADVTPGMQVVREEIFGPVLVATPFDDLDEAVRLANDSIYGLGASIWSNDLRQVMDLVPRIKAGTVWVNAHNLLDPSMPFGGFKQSGIGREMGHAAIEAYTENKSVCIAY

SEQ ID NO: 81 - protein, 504 aa, Ruegeria pomeroyi
MQVARPDSLVSQILPAVADYLDSPAKLLLGGTSTAASDGRTMDVFNPATGKKLAEVPWGGAAEIDLAVKAAQAALEGDWSRMRPVERQRVLLNLADLIEANGEELAQLETLNNGKSVMLSRLVEVGNSSNYLRYMAGWSTKIEGSTIDVSIAVPPGAKYQAYTRKEPVGVVGAITPWNFPLNMAIWKLAPALACGNTVVLKPAEETPLTSLRLGELCLEAGLPPGVVNVVSGTGAEAGAALTAHPGVNKLTFTGSTEVGKIIGIQAMRDMKRVTLELGGKAPMVMFDDMDLDQLSEAARIGILFNSGQTCCAGTRIYAQRGIYDRICETMANVVGALSVGSGLDPANAINPMVSAKHQAHVSACIAGGVEEGATPLLDTGAYDGEGYFVRPQIFTDVRQDMRIMQDEVFGPVFTITPFDDPDEAIRMANDTRYGLGASIWTTNLNTMHRYVPQLQAGTVWVNSHNVPDANMPFGGYKQSGIGREHGRAALDAYLETKSVCIAVR

SEQ ID NO: 82 - protein, 499 aa, Oceanospirillum sp. MED92
MSTATFSPSEAAQAFLQRQHKILIGAEWQAAQDGRTLDVVNPADGEKIATVPSGGAEDIDRAVSAAKQAFEDSEWSRIKPVDRQKLLWDFADLIEKNAPLLAELEALDNGKSVVIAEHVDIRLAVDFLRYMAGFATKIEGRSVDVSVPFMPDAQFHGYTRREAVGVVGAIVAWNFPLLLACWKLGPALATGCTVVLKPAEDTPLTALKLAELALEAGYPPGVFNVVTGLGHEAGAALSSHPDVDKLTFTGSTEVGKLIGKAAMDSMTRVTLELGGKSPTIVLQDADLQNAAAGAANAIFFNQGQVCCAGSRLYVHKKHFDNVVADISDIANGMTLGAGLDPNAQMGPLVSAKQQQRVCGYIDQGITSGAKVVAGGSAADGPGFFVKPTVMVDVDHNASVVKEEIFGPVLVAMPFDDIDEAVRIANDSQYGLGASIWSNNLSEVHRMIPRIKSGSVWVNCHTALDPALPFGGYKQSGLGREMGSDVIEHYTEVKSVLMSI

SEQ ID NO: 83 - protein, 495 aa, Burkholderia ambifaria
MALVRMLDEVRAFLAREHGHYIDGRAVAGRGERIDVRDPATRAVIGSVAQATDDDVEAAIASSHRAFRSEWANRTPADRERILLRFADLIEAHGEEIAQIETAQSGKLIGLSRVIEVGWSARWLRYYAGWATKIAGETLAPSFPGMNGERYTSFTLREPLGVVFGIIPWNFPVMIPVWKFGAALATGNTVLIKSSEFTPLTMLRIAELATEAGLPAGALNVINGTGQVGAKVIGDPRVAKVSFTGSVPTGRIIGEQAVNANLTRFTLELGGKNAAAFLADTPVDKILDGIVEAGFLHSGQVCASAERFFVHRSKFDEVVEKMKARLDSFQPADPMDDAGMIGPVCNEPQFRKCVDAFDLARAEGDTIVTGGGAYARDGFYVKPTIVLPRSLESASYRKEIFGPVGAFVPFDDEEELIAMINDTPFGLTASLWTNDLSKALRYVPRIEAGTVWVNMHTLVDPAVPFGGAKGSGVGREYGSSFIDAYTEPKAVTIRF

SEQ ID NO: 84 - protein, 502 aa, Azospirillum sp. B510
MKIAQPDSIVEQQAPAVADFLKAPLMVIGAASVPAKSGRTYSVYNPATGKPLAEVPAGSAEDVDAAVKAAQAAFEGPWSRMLPTQRQAAILRLADLIEANGEELAQLETLNNGKSIMMSRLLEAQGAAEYFRYMAGWATKIEGATLDVSIPIPPGMSYQAYTRKEPVGVVAAITPWNFPLTMAAWKVAPALAAGCTVVLKPAEETPLTSIRLAQLCLEAGIPEGVVNVVTGLGEAAGAPLVAHPGIAKISFTGSTETGKLIGIQAMRDMKRVTLELGGKAPMVMFDDMDLDLLGVAAGIGSFFNTGQTCCAGVRIYAQKGVYDRVLDTIAAVTRSLSIGSGLDPRNQINPLVSARHQAHVRSCIARGIEDGAKPVIKGSAPADGFYVAPELFVDVRQDMALMQEEVFGPVVTVTPFDDPDEAIRLANDTRFGLGASIWTTDINKMMRYVPKIQAGTVWVNAHNLPDQNMPFGGFKQSGVGREHGRGALDNYLETKSVCVAFR

SEQ ID NO: 85 - protein, 497 aa, Xenorhabdus nematophila
MSEINLLGSVTAFLQRTHGHYINGVSVPGQGNETFSVVNPASGETIATVNQGEEADINQAMQAASDAFHGAWARTSPLERGNCLNRLADLLQENGEELAQLESLCSGKPIQLSRMLEVGASADYLRYFAGWSSKISGETLNVSLPSLKGEKYTAFTRREPIGVVVGIIPWNFSIMIAIWKLGAALASGCTIVLKPSEYTPLIILRVAELAKEAGIPDGVINIINGSGSRVGSALISHPQCSKVTFTGSVPTGMIVGKSALEQGLKHTTLELGGKNAAAFLSDMTVEKIVDGILEAGYVYQGQICAAAERFYIPSIHMDAVLELLSERLSAMKIGSPLDESTEMGPLANKQHYEKILSLFEQARQDGCEIVYGGYALEGAGFFVAPTIVRANSPEDTLMKEETFGPIGTFLSYDDEEELIGMMNSTPFGLSASLWTNDLSKAMRMIPRIESGILWINMHTYLDPSVPFGGMKSSGIGREFGSAFIEHYTELKSVMMRY

SEQ ID NO: 86 - protein, 501 aa, Burkholderia pseudomallei
MNLAALSTQHQRQSGFLARRQFGNWIDGRAAEPRSGRYLPVVDPATEMTIAEVAASDARDVDAAVAAARRVFDSGDWPRMRPASREKLLHQLAERLERYADELAALETLETGKLIGVARAIDVLGGAEYVRYMAGWATKLEGSTLDTSIAAPAGAEYFAYTRREAVGVVGAIVPWNFPLAIALWKVATALACGCTVVLKPSEETPLTALRLGELAQEAGLPDGVLNIVTGAGAEAGAALAAHPGIDKITFTGSVGVGRAIGHAAVERMARFTLELGGKSPLIVLDDADPDFAAHGAAQGIFFNQGQVCTAGSRVYVQKRLFERVVAGIAAAAEAMKIGSGFDPNTQIGPLVSKRHFERVLGHVDAAKEEGATLVTGGTRALDGGYFVKPTVFVDAAPAMRIVREEVFGPVVTVTPFDTVDDAVRLANDTDFGLAASVWSQNLSHVHRVVPRLKAGIVWVNTHNMLDPNLPFGGFKQSGYGRELGRAALEQFTELKSVCIAH

SEQ ID NO: 87 - protein, 501 aa, Burkholderia mallei
MNLAALSTQHQRQSGFLARRQFGNWIDGRAAEPRSGRYLPVVDPATEMTIAEVAASDARDVDAAVAAARRAFDSGDWPRMRPASREKLLHQLAERLERYADELAALETLETGKLIGVARAIDVLGGAEYVRYMAGWATKLEGSTLDTSIAAPAGAEYFAYTRREAVGVVGAIVPWNFPLAIALWKVATALACGCTVVLKPSEETPLTALRLGELAQEAGLPDGVLNIVTGAGAEAGAALAAHPGIDKITFTGSVGVGRAIGHAAVERMARFTLELGGKSPLIVLDDADPDFAAHGAAQGIFFNQGQVCTAGSRVYVQKRLFERVVAGIAAAAEAMKIGSGFDPNTQIGPLVSKRHFERVLGHVDAAKEEGATLVTGGTRALDGGYFVKPTVFVDAAPAMRIVREEVFGPVVTVTPFDTVDDAVRLANDTDFGLAASVWSQNLSHVHRVVPRLKAGIVWVNTHNMLDPNLPFGGFKQSGYGRELGRAALEQFTELKSVCIAH

SEQ ID NO: 88 - protein, 495 aa, Burkholderia ambifaria
MALVRMLDEVRAFLAREHGHYIDGRAVAGRGERIDVRDPATRAVIGSVAQATDDDVEAALASSHRAFRGEWANLTPADRERILLRFADLIEAHGEEIAQIETAQSGKLIGLSRVIEVGWSARWLRYYAGWATKIAGETLAPSFPSMNGERYTSFTLREPLGVVFGIIPWNFPVMIPVWKFGAALATGNTVLIKSSEFTPLTMLRIAELATEAGLPAGTLNVINGTGQVGAKVIGDPRVAKVSFTGSVPTGRIIGEQAVNANFTRFTLELGGKNAAAFLADTPVDKILDGIVEAGFLHSGQVCASAERFFVHRSKFDEVVEKMKARLDGFQPADPMDDAGMIGPVCNEPQFRKCVDAFDVARAEGDTIVTGGGAYARDGFYVKPTIVLPRSLESASYRKEIFGPVGAFVPFDDEEELIAMMNDTPFGLTASLWTNDLSKALRYVPRIEAGTVWVNMHTLVDPAVPFGGAKGSGIGREYGSSFIDAYTEPKAVTIRF

SEQ ID NO: 89 - protein, 495 aa, Burkholderia ambifaria
MALVRMLDEVRAFLAREHGHYIDGRAVAGRGERIDVRDPATRAVIGSVAQATDDDVEAALASSHRAFRGEWADLTPADRERILLRFADLIEAHGEEIAQIETAQSGKLIALSRVIEVGWSARWLRYYAGWATKIAGETLAPSFPSMNGEHYTSFTLREPLGVVFGIIPWNFPVMIPVWKFGAALATGNTVLIKSSEFTPLTMLRIAELATEAGLPAGTLNVINGTGQVGAKVIGDPRVAKVSFTGSVPTGRIIGEQAVNANFTRFTLELGGKNAAAFLADTPVDRILDGIVEAGFLHSGQVCASAERFFVHRSKFDEVVEKMKARLDAFQPADPMDDAGMIGPVCNEPQFRKCVDAFDLARAEGDTIVTGGGAYARDGFYVKPTIVLPRSLESASYRKEIFGPVGAFVPFDDEEELIAMMNDTPFGLTASLWTNDLSKALRYVPRIEAGTVWVNMHTLVDPAVPFGGAKGSGIGREYGSSFIDAYTEPKAVTIRF

SEQ ID NO: 90 - protein, 495 aa, Pseudomonas putida
MSDITLLPAVTAFLAREHGVFIHGQHLASQSSSTIAVVNPANGQTIAHIADANQADVDHAVSSSRQGFATWSHTSPAARAAVLFKLADLLEAHREELAQLETVQSGKLIGISRAFEVEQAAHFLRYYAGWATKITGQTITPSLPSFAGERYSAFTLREPIGVVVGIVPWNFATMIAIWKLASALTTGCSIILKPSEFTPLTLLRIAELATQAGLPAGALNVLTGGGLVGKALIEHAGTDKVSFTGSVPTGIAVGQAAMGAKLTRATLELGGKNAVAFLPDVATDKAVDGIIEAGFLHSGQICAAGERFYVHRSRIDPLLDALSQRLGQLKIGSPLDESTQFGPVANKPHQQKLAELFATARAEGSQIIHGGKLGDGPGCFVEPTVILARSANDTLLNQETFGPVATFLPYDDEDELLQLMNASPYGLSASLWTNDLGKAMRMIPQIQAGTLWVNMHTLLDPAVPFGGIKASGVGREFGSAFIDDFTELKSVMIRY

SEQ ID NO: 91 - protein, 501 aa, Burkholderi pseudomallei
MNLAALSTQHQRQSGFLARRQFGNWIDGRAAEPRSGCYLPVVDPATEMTIAEVAASDARDVDAAVAAARRAFDSGDWPRMRPASREKLLHQLAERLERYADELAALETLETGKLIGVARAIDVLGGAEYVRYMAGWATKLEGSTLDTSIAAPAGAEYFAYTRREAVGVVGAIVPWNFPLAIALWKVATALACGCTVVLKPSEETPLTALRLGELAQEAGLPDGVLNIVTGAGAEAGAALAAHPGIDKITFTGSVGVGRAIGHAAVERMARFTLELGGKSPLIVLDDADPDFAAHGAAQGIFFNQGQVCTAGSRVYVQKRLFERVVAGIAAAAEAMKIGSGFDPNTQIGPLVSKRHFERVLGHVDAAKEEGATLVTGGTRALDGGYFVKPTVFVDAAPAMRIVREEVFGPVVTVTPFDTVDDAVRLANDTDFGLAASVWSQNLSHVHRVVPRLKAGIVWVNTHNMLDPNLPFGGFKQSGYGRELGRAALEQFTELKSVCIAH

SEQ ID NO: 92 - protein, 495 aa, Burkholderia ambifaria
MALVRMLDEVRAFLAREHGHYIDGRAVAGRGERIDVRDPATRAVIGSVAQATDDDVEAALASSHRAFRGEWANLTPADRERILLRFADLIEAHGEEIAQIETAQSGKLIGLSRVIEVGWSARWLRYYAGWATKIAGETLAPSFPSMNGERYTSFTLREPLGVVFGIIPWNFPVMIPVWKFGAALATGNTVLIKSSEFTPLTMLRIAELATEAGLPAGTLNVINGAGQVGAKVIRDPRVAKVSFTGSVPTGRIIGEQAVNANFTRFTLELGGKNAAAFLADTPVDKILDGIVEAGFLHSGQVCASAERFFVHRSKFDEVVEKMKARLDAFQPADPMDDAGMIGPVCNEPQFRKCVDAFDLARSEGDTIVTGGGAYARDGFYVKPTIVLPRSLESASYRKEIFGPVGAFVPFDDEEQLIAMMNDTPFGLTASLWTNDLSKALRYVPRIEAGTVWVNMHTLVDPAVPFGGAKGSGIGREYGSSFIDAYTEPKAVTIRF

SEQ ID NO: 93 - protein, 500 aa, Mesorhizobium opportunistum
MNERTLPPISPAAAAFLARPHRPFINGRFVDGLAGDGLAVDDSASGEIVAHVPESGPELVDQAVRAARAALEGPWASMRPVDRQNLMLKLADAVEADADLLAEIESIENGKSLGVARMLSAAGTVDWLRYYAGWATKIEGSTFQVSIPVPPGAKHQAMTVMEPVGVVGAIVPWNFPLLIGMWKIAPALACGCTVVLKPPQETPLGLLRLAELIEASGFPPGVVNIVTGSGSVTGEALIRHPGIDKLTFTGSTEVGKRVGHAAVDRVARFTLELGSKSPMILLADMEEGIEPLVAGLGMFFNQGQVCTSASRLLIEKSIYDRTLARLAEIADGMTLGAGRDADAQINPLVSAKHKRSVEGFVERGLAAGVERVSGARPVPARGHYVAPTILHNVRPDMEIVREEVFGPVVAAMPVADLDEAIRIANDTRYGLSASIWTRDMGKAMTAIHGLKAGTVWVNSHNTLDPNAPFGGFKQSGIGREHGRAAIDGYLETKTVIMRYA

SEQ ID NO: 94 - protein, 496 aa, Candidatus Koribacter versatilis
MSVVSAVELNSNVSQFITKPRKMLIGGNWIDSASGKFFETLNPATGEVLARVAEGDRADIDLAVAAARKAFESGPWSKMSPSQRGRLLWKLADLLEQHLEEFAELESLDNGKPLSVARVADVPLAVDLFRYMAGWATKVEGNTIPLGPQFHAYTYREPVGVIGQIIPWNFPLLMAAWKLGPALAVGCTVVLKPAEQTPLSALRLGELIMEAGFPDGVVNVVPGFGETAGAALAAHPDVDKIAFTGSTEVGKLIVQAAAGNLKKVSLELGGKSPNIVLADADLDIAISGSANAIFFNHGQCCCAGSRLFVHKSQFDKVVEGVAEAAKNIRLGSGLDPATNMGPLVSQEQLDRVCGYLESGVQQGAKPLVGGKKQTGPGYFVEPTVLVDVKPTMKVVCEEIFGPVVTAIPFNSVDEVLNSANASSYGLAAAVWTRDINKAHSLAAKLRAGTVWVNCYNVFDAALPFGGYKQSGWGREMGHDALELYTETKAVCVRLEN

SEQ ID NO: 95 - protein, 501 aa, Burkholderia mallei
MNLAALSTQHQRQSGFLARRQFGNWIDGRAAEPRLGRYLPVVDPATEMTIAEVAASDARDVDAAVAAARRAFDSGDWPRMRPASREKLLHQLAERLERYADELAALETLETGKLIGVARAIDVLGGAEYVRYMAGWATKLEGSTLDTSIAAPAGAEYFAYTRREAVGVVGAIVPWNFPLAIALWKVATALACGCTVVLKPSEETPLTALRLGELAQEAGLPDGVLNIVTGAGAEAGAALAAHPGIDKITFTGSVGVGRAIGHAAVERMARFTLELGGKSPLIVLDDADPDFAAHGAAQGIFFNQGQVCTAGSRVYVQKRLFERVVAGIAAAAEAMKIGSGFDPNTQIGPLVSKRHFERVLGHVDAAKEEGATLVTGGTRALDGGYFVKPTVFVDAAPAMRIVREEVFGPVVTVTPFDTVDDAVRLANDTDFGLAASVWSQNLSHVHRVVPRLKAGIVWVNTHNMLDPNLPFGGFKQSGYGRELGRAALEQFTELKSVCIAH

SEQ ID NO: 96 - protein, 501 aa, Burkholderia multivorans
METNDNFALLDATRAFLAKPKRMLIGGEWSDAASGRTIEVVNPADGSTIACVPEADEHDVQRAVAAARRAFDTGPWRAAKTTDRERLLLTLADLIDANARELAEIESLDNGKSVSVAQGLDVAMAAQCFRYMAGWATKIEGSVVDAGMPYLPGSETFTYTRKEPIGVVGAIIPWNFPLLMAAWKLAPALATGCTVVLKPAEDTPLTALRLGELIGDAGFPDGVVNIVTGYGHTAGAALSRDPRIDKIAFTGSTQTGKAIGHAALDNMTRMSLELGGKSPVIVLPDVDVDKAAEGIANAIFFNQGQVCTAGSRAYVHTKVFDRVMERVAQIAAGLKIGPGMDPATQIGPLVSAKQRARVCDYIASGFEDGARAIAGGRARDGAGFFVEPTVLVDTTHAMRVVREEIFGPVLVAMPFDDIDTAVQLANDTPYGLGASIWSNDLSAVHKLVPRIAAGTVWVNCHSLLDNAMPFGGMKQSGFGRELGRAVIDQYTETKSVMMNYA

SEQ ID NO: 97 - protein, 501 aa, Burkholderia pseudomallei
MNLAALSTQHQRQSGFLARRQFGNWIDGRAAEPRSGRYLPVVDPATEMTIAEVAASDARDVDAAVAAARRAFDSGDWPRMRPASREKLLHQLAERLERYADELAALETLETGKLIGVARAIDVLGGAEYVRYMAGWATKLEGSTLDTSIAAPAGAEYFAYTRREAVGVVGAIVPWNFPLAIALWKVATALACGCTVVLKPSEETPLTALRLGELAQEAGLPDGVLNIVTGAGAEAGAALAAHPGIDKITFTGSVGVGRAIGHAAVERMARFTLELGGKSPLIVLDDADPDFAAHGAAQGIFFNQGQVCTAGSRVYVQKRLFERVVAGIAAAAEAMKIGSGFDPNTQIGPLVSKRHFERVLGHVGAAKEEGATLVTGGTRALDGGYFVKPTVFVDAAPAMRIVREEVFGPVVTVTPFDTVDDAVRLANDTDFGLAASVWSQNLSHVHRVVPRLKAGIVWVNTHNMLDPNLPFGGFKQSGYGRELGRAALEQFTELKSVCIAH

SEQ ID NO: 98 - protein, 501 aa, Burkholderia pseudomallei
MNLAALSTQHQRQSGFLARRQFGNWIDGRAAEPRSGRYLPVVDPATEMTIAEVAASDARDVDAAVAAARRAFDSGDWPRMRPASREKLLHQLAERLERYADELAALETLETGKLIGVARAIDVLGGAEYVRYMAGWATKLEGSTLDTSIAAPAGAEYFAYTRREAVGVVGAIVPWNFPLAIALWKVATALACGCTVVLKPSEETPLTALRLGELAREAGLPDGVLNIVTGAGAEAGAALAAHPGIDKITFTGSVGVGRAIGHAAVERMARFTLELGGKSPLIVLDDADPDFAAHGAAQGIFFNQGQVCTAGSRVYVQKRLFERVVAGIAAAAEAMKIGSGFDPNTQIGPLVSKRHFERVLGHVGAAKEEGATLVTGGTRALDGGYFVKPTVFVDAAPAMRIVREEVFGPVVTVTPFDTVDDAVRLANDTDFGLAASVWSQNLSHVHRVVPRLKAGIVWVNTHNMLDPNLPFGGFKQSGYGRELGRAALEQFTELKSVCIAH

SEQ ID NO: 99 - protein, 493 aa, Marinomonas sp. MWYL1
MSDIPLLDSTQRFLQQDHGQFINGQTKASGDNTFDIINPSTEAVIAKIHSATTQEVDAAIESSYQVFKGAWGKTSPYIRGVVLNKLADLIEQNGEEIAQLETLCSGKSIHLSRMFEVQQSAMFLRYFAGWSTKINGETMTPSFPSMQGEEYSAFTRREAIGVVAGILPWNFSVMIACWKIGAALCTGCTIVLKPSEFTPLTILRIAELAKEAGVPDGVLNIVNGKGDVGGQLIQHPKVRKVSFTGSVATGKKISAAASADLTRCTLELGGKNTAAILKDADIDRVVGGLFQLGYIHQGQVCAAPERVYVHSSRIDELTTKLAQKLSEAKIGSPLDESVYFGPLSNEPQFNKVCEYLEIAHKESRVLHGGKAISGKGFFVEPTIVQASSVDETLMQEETFGPIISFMPYEDEEELIDLINNTPFGLSSSIWTNNLSQAMRMIPKIESGTVWVNMHSILDPSVPFGGTKQSGVGREFGREFINDYTEVKSVIMCY

SEQ ID NO: 100 - protein, 501 aa, Burkholderia multivorans
METNDNFALLDATRAFLAKPKRMLIGGEWSDAASGRTIEVVNPADGSTIACVPEADQHDVQRAVAAARRAFDAGPWRAAKTTDRERLLLTLADLIDANARELAEVESLDNGKSVIVAQGLDVAMAAQCFRYMAGWATKIEGSVVDAGMPYLPGSETFTYTRKEPVGVVGAIIPWNFPLLMAAWKLAPALATGCTVVLKPAEDTPLTALRLGELIGEAGFPDGVVNIVTGYGHTAGAALSRDPRIDKIAFTGSTQTGKAIGHAALDNMTRMSLELGGKSPVIVLPDVDVDKAAEGIANAIFFNQGQVCTAGSRAYVHTKVFDRVMERVAQIAAGLKIGPGMDPATQIGPLVSAKQRARVCDYIASGFEDGARAIAGGRARDGAGFFVEPTVLVDTTHAMRVVREEIFGPVLVAMPFDDIDTAVQLANDTPYGLGASIWSNDLSAVHKLVPRIAAGTVWVNCHSLLDNAMPFGGMKQSGFGRELGRAVIDQYTETKSVMMNYA

SEQ ID NO: 101 - protein, 501 aa, Burkholderia multivorans
METNDNFALLDATRAFLAKPKRMLIGGEWSDAASGRTIEVVNPADGSTLARVPEADEHDVQRAVAAARRAFDTGPWRAAKTTDRERLLLTLADLIDANARELAEIESLDNGKSVSVAQGLDVAMAAQCFRYMAGWATKIEGSVIDAGMPYLPGSETFTYTRKEPVGVVGAIIPWNFPLLMAAWKLAPALATGCTVVLKPAEDTPLTALRLGELIGDAGFPDGVVNIVTGYGHTAGAALSRDPRIDKIAFTGSTQTGKAIGHAALDNMTRMSLELGGKSPVIVLADVDVDKAAEGIANAIFFNQGQVCTAGSRAYVHTKVFDRVMERAAQIAAGLKIGPGMDPATQIGPLVSAKQRARVCDYIASGFEEGARAIAGGRARDGAGFFVEPTVLVDTTHAMRVVREEIFGPVLVAMPFDDIDTAVRLANDTPYGLGASIWSNDLSAVHKLVPRIAAGTVWVNCHSLLDNAMPFGGMKQSGFGRELGRAVIDQYTETKSVMMNYA

SEQ ID NO: 102 - protein, 495 aa, Pseudomonas aeruginosa
MSIAIDPSVTAFLRSHHGLLIDGESRPARSGADMPLYDPASGAELARVARAAADDVDLAVAAARRAFEGSWAQQRPADRERLLLCLAERLEAHGEQLAQLETLNNGKSINLSRALEVGASVEFIRYMAGWATKIEGRSLDLSIAAVPGARYRAYTVPEPVGVVGAIVPWNFPLLMAVWKIVPALACGCTVVLKPADETPLTALRLGQLCLEAGIPPGVVNIVTGTGAEAGAALAAHPGIDKLAFTGSTPVGKLIGHAAVENMTRFSLELGGKSPVIILDDTSLDMAAAGSAAAIFFNQGQVCTAGSRLYVQRKRFEQVLERLASIAADLNIGPGLDPAAQINPLVSARQQGRVLGMIEGGVAEGARVVCGGARAGETGFYVQPTVLADVTPRMQVVREEIFGPVLVATPFDDLDEAVRLANDSIYGLGASIWSNDLRQVMDLLPRIKAGTVWVNTHNMLDPSMPFGGFKQSGIGREMGHAAIEAYTENKSVCIAY

SEQ ID NO: 103 - protein, 502 aa, Burkholderia sp. CCGE1003
MNDNSRPLDMLDSTRTFLAAPKRMFIDGEWRASASGATLDVLNPADGSLLAQVPSADEADVDLAVQAARRAFDDSAWSRMKPTDRERILLRVAELIEANARELAEIESLDNGKPVAVAQGLDVSMAAQCFRYMAGWATKIEGSTLDAALPYSPSNAFFAYTRKEAVGVVGAIIPWNFPLLMASWKLAPALATGCTVVLKPAEDTPLSALRLATLLSEAGLPKGVVNIVTGYGRSAGAALARHPGIDKIAFTGSTQTGKAIGHAALDNMTRMSLELGGKSPVIVLPDVDIERAAEGVANAIFFNSGQVCTAGSRVYVHETVFDRVMERVAAIAEALPVGPGLDANTQIGPLVSARQMDRVLGYIEAGRDEGARAIAGGAREGGAGFFVKPTVLVDTDHSMRVVREEIFGPVLVAMPFKDIDSAVAQANDTPYGLGASIWSNNLSAIHNLIPRIKAGTVWVNCHSLLDNAMPFGGVKQSGFGRELGRAVIDMYTEMKSVLINHA

SEQ ID NO: 104 - protein, 495 aa, Acinetobacter sp. SH024
MSEVQILESVQQFMARQHGHFIDGKLVAAELLDKVDIVNPSTEEVVAQISIGSQQDVDSAVKSAEHAFQNAWAETTPYERGVKLNKLADLIEQYGEELAQLETLSTGKLINISRHLEVAQSVIFLRYFAGWATKINGQTMQPSIPSMQGEKYTAFTLRQPIGVVAGIVPWNFSLMIGVWKIGSALTTGCTIVLKPSEFASLSLLRLAELAIEAGIPVGVINVVTGKGETGQYLIESPLVKKVSFTGSVPTGIAIGKLAMSSDLTRVSLELGGKNAIAVLADANIDEILPTLLQATFVHQGQVCASPERFFVHRAKYDELVEKLSKALSSFKIGSAMDEGSMFGPLSNQPHFHKVKHYLDMAKAKNQIIAGGETLDRSGYFVQPTLISFKNTDDPLFSEETFGPVVGIMPFETDEELVQLMNQSRFGLTASIWTNDLSKALRLIPKIEAGTLWVNMHTFLDPSVPFGGVKASGIGREFSDAFIEDYTELKSVMIRY

SEQ ID NO: 105 - protein, 495 aa, Acinetobacter sp. RUH2624
MSEVQILQNVQQFMARQHGHFIDGKLVAAEHLDKVDIVNPSTEQVVAQISIGSQQDVASAVKSAKHAFQNAWAETTPYERGVKLNKLADLIEQHGEELAQLETLSTGKLINISRHLEVAQSVIFLRYFAGWATKINGQTMQPSIPSMQGEKYTAFTLRQPVGVVAGIVPWNFSLMIGIWKIGSALTTGCTIVLKPSEFASLSLLRLAELAIEAGIPAGVINVVTGKGDTGQYLIESPLVKKVSFTGSVPTGIAIGKLAMSSDLTRVSLELGGKNAIAVLADANIDEILPTLLQATFVHQGQVCASPERFFVHHTKHNELVEKLSKALSSLKIGSAMDEGSMFGPLSNQPHFHKVKHYLDMAKANNQIIAGGEALDRSGYFVQPTLISFKNTDDPLFSEETFGPVVGVMPFETDEELIQLMNQSRFGLTASIWTNDLSKALRLIPKIEAGTLWVNMHTFLDPSVPFGGVKASGIGREFSDAFIEDYTELKSVMIRY

SEQ ID NO: 106 - protein, 495 aa, Acinetobacter baumanni
MSEVQILESVQQFIARQHGHFIDGKLVAAELLDKVDIVNPSTEQVVAQISIGSQQDVESAVKSAEHAFQNAWAETTPYERGVKLNKLADLIEQHGEELAQLETLSTGKLINISRHLEVAQSVIFLRYFAGWATKINGQTMQPSIPSMQGEKYTAFTLRQPVGVVAGIVPWNFSLMIGVWKIGSALTTGCTIVLKPSEFASLSLLRLAELAIEAGIPAGVINVVTGKGETGQYLIESPLVKKVSFTGSVPTGIAIGKLAMSSDLTRVSLELGGKNAIAVLADANIDEILPTLLQATFVHQGQVCASPERFLVHRTKYDELVDKLSKALSQFKIGSAMDEGSMFGPLSNQPHFHKVKHYLDMAKANNQIIAGGEALDQTGYFVQPTLISFKNTDDPLFSEETFGPVVGVMSFDTDEELIQLMNQSRFGLTASIWTNDLSKALRLIPKIEAGTLWVNMHTFLDPSVPFGGVKASGIGREFSDAFIEDYTELKSVMIRY

SEQ ID NO: 107 - DNA, 52 bp, artificial
  synthetic oligonucleotide primer
atgatcgctagcaggagaaattaactatgttgacaaaagcaacaaaagaaca

SEQ ID NO: 108 - DNA, 40 bp, artificial
  synthetic oligonucleotide primer
gactatgctcagcttagagagctttcgttttcatgagttc

SEQ ID NO: 109 - DNA, 40 bp, artificial; identical to SEQ ID NO: 108
  synthetic oligonucleotide primer

SEQ ID NO: 110 - DNA, 42 bp, artificial
  synthetic oligonucleotide primer
tcctctaaatctctagaaagggtgccggcagcttgatatgtt

SEQ ID NO: 111 - DNA, 34 bp, artificial
  synthetic oligonucleotide primer
atgatcggtaccatgcctaagtaccgttccgcca

SEQ ID NO: 112 - DNA, 37 bp, artificial
  synthetic oligonucleotide primer
atgatcgctagcttaaccccccagtttcgatttatcg

SEQ ID NO: 113 - DNA, 40 bp, artificial
  synthetic oligonucleotide primer
gactatggtaccatgtatacagtaggagattacctattag

SEQ ID NO: 114 - DNA, 39 bp, artificial
  synthetic oligonucleotide primer
gactatgcatgcttatgatttattttgttcagcaaatag

SEQ ID NO: 115 - DNA, 39 bp, artificial
  synthetic oligonucleotide primer
gactattctagattatgatttattttgttcagcaaatag

SEQ ID NO: 116 - DNA, 52 bp, artificial
  synthetic oligonucleotide primer
gactatgcatgcaggagatataccatgcaacataagttactgattaacggag

SEQ ID NO: 117 - DNA, 40 bp, artificial
  synthetic oligonucleotide primer
gactattctagattaatgtttaaccatgacgtggcggacg

SEQ ID NO: 118 - DNA, 48 bp, artificial
  synthetic oligonucleotide primer
gactatgcatgcaggagatataccatgaccaataatcccccttcagca

SEQ ID NO: 119 - DNA, 39 bp, artificial
  synthetic oligonucleotide primer
gactattctagattagaacagccccaacggtttatccga

SEQ ID NO: 120 - DNA, 53 bp, artificial
  synthetic oligonucleotide primer
gactatgcatgcaggagatataccatgaattttcatcatctggcttactggca

SEQ ID NO: 121 - DNA, 60 bp, artificial
  synthetic oligonucleotide primer
ggcttatccagatggttttcagttcagtgaatttttcaagggcgtgcagggatttgtcgc

SEQ ID NO: 122 - DNA, 50 bp, artificial
  synthetic oligonucleotide primer
gactattctagattaggcctccaggcttatccagatggttttcagttcag

SEQ ID NO: 123 - DNA, 52 bp, artificial
  synthetic oligonucleotide primer
gactatgcatgcaggagatataccatgaaacttaacgacagtaacttattcc

SEQ ID NO: 124 - DNA, 38 bp, artificial
  synthetic oligonucleotide primer
gactattctagattaaagaccgatgcacatatatttga

SEQ ID NO: 125 - DNA, 48 bp, artificial
  synthetic oligonucleotide primer
ctagtagcatgcaaggagatataccatgacagagccgcatgtagcagt

SEQ ID NO: 126 - DNA, 40 bp, artificial
  synthetic oligonucleotide primer
gactattctagattaataccgtacacacaccgacttagtt

SEQ ID NO: 127 - DNA, 51 bp, artificial
  synthetic oligonucleotide primer
gactatgcatgcaggagatataccatggctaacgtgacttatacggatacg

SEQ ID NO: 128 - DNA, 34 bp, artificial
  synthetic oligonucleotide primer
gactattctagattagaccgccatcaccgtcacc

SEQ ID NO: 129 - DNA, 45 bp, artificial
  synthetic oligonucleotide primer
gactatgcatgcaggagatataccatgcccctcacaggcaacctg

SEQ ID NO: 130 - DNA, 38 bp, artificial
  synthetic oligonucleotide primer
gactattctagattagtcttcccgtttaccatcaagca

SEQ ID NO: 131 - DNA, 35 bp, artificial
  synthetic oligonucleotide primer
gactatggatccatgacagagccgcatgtagcagt

SEQ ID NO: 132 - DNA, 40 bp, artificial
  synthetic oligonucleotide primer
gactatggatccttaataccgtacacacaccgacttagtt

SEQ ID NO: 133 - DNA, 21 bp, artificial
  synthetic oligonucleotide primer
ttgcttacgccacctggaagt

SEQ ID NO: 134 - DNA, 22 bp, artificial
  synthetic oligonucleotide primer
gaacggtcgcatgagcagaaag

SEQ ID NO: 135 - DNA, 21 bp, artificial
  synthetic oligonucleotide primer
tgacgataatttctggcaagc

SEQ ID NO: 136 - DNA, 21 bp, artificial
  synthetic oligonucleotide primer
gcaggctgacattaagttcgt

SEQ ID NO: 137 - DNA, 20 bp, artificial
  synthetic oligonucleotide primer
agatttggcctgcggtgaaa

SEQ ID NO: 138 - DNA, 24 bp, artificial
  synthetic oligonucleotide primer
ctgttagttgttatttattggcgg

SEQ ID NO: 139 - DNA, 22 bp, artificial
  synthetic oligonucleotide primer
catttattgcgcgacgcattat

SEQ ID NO: 140 - DNA, 22 bp, artificial
  synthetic oligonucleotide primer
atagcgggcttttaacttgagg

SEQ ID NO: 141 - DNA, 22 bp, artificial
  synthetic oligonucleotide primer
cactgaagaggtatgcggaaaa

SEQ ID NO: 142 - DNA, 21 bp, artificial
  synthetic oligonucleotide primer
ctgggcattttatgccggtag

SEQ ID NO: 143 - DNA, 48 bp, artificial
  synthetic oligonucleotide primer
ctagtagagctcaaggagatataccatgacagagccgcatgtagcagt

SEQ ID NO: 144 - DNA, 40 bp, artificial
  synthetic oligonucleotide primer
gactatgagctcttaataccgtacacacaccgacttagtt

SEQ ID NO: 145 - DNA, 54 bp, artificial
  synthetic oligonucleotide primer
tgcatcgaattcaggagaaattaactatgaacgagtacgcccccctgcgtttgc

SEQ ID NO: 146 - DNA, 36 bp, artificial
  synthetic oligonucleotide primer
tgcatcaagctttcagatatgcaaggcgtggcccag

SEQ ID NO: 147 - DNA, 52 bp, artificial
  synthetic oligonucleotide primer
gggcccaagcttaggagaaattaactatgatgaacttcaacaatgttttccg

SEQ ID NO: 148 - DNA, 38 bp, artificial
  synthetic oligonucleotide primer
gggccctctagattatgagtcatgatttactaaaggct

SEQ ID NO: 149 - DNA, 55 bp, artificial
  synthetic oligonucleotide primer
gggcccaagcttaggagaaattaactatgatgagtcaggcgctaaaaaatttact

SEQ ID NO: 150 - DNA, 38 bp, artificial
  synthetic oligonucleotide primer
gggccctctagattaattgtgattacgcatcacccctt

(19) United States
(12) Patent Application Publication
(10) Pub. No.: US 2014/0065697 A1
(43) Pub. Date: Mar. 6, 2014

(54) CELLS AND METHODS FOR PRODUCING ISOBUTYRIC ACID

(71) Applicants: Kechun Zhang, Roseville, MN (US); Mingyong Xiong, Saint Paul, MN (US); Adam P. Woodruff, Minneapolis, MN (US)

(72) Inventors: Kechun Zhang, Roseville, MN (US); Mingyong Xiong, Saint Paul, MN (US); Adam P. Woodruff, Minneapolis, MN (US)

(73) Assignee: Regents of the University of Minnesota, Saint Paul, MN (US)

(21) Appl. No.: 13/984,502

(22) PCT Filed: Feb. 10, 2012
(86) PCT No.: PCT/US12/24640

§ 371 Date: Nov. 8, 2013

Related U.S. Application Data
(60) Provisional application No. 61/441,939, filed on Feb. 11, 2011.

(51) Int. Cl. C12N 15/70

(52) U.S. Cl. CPC C12N 15/70
USPC 435/252.33

(57) ABSTRACT

Disclosed herein are cells and methods for renewably producing isobutyrate. In some cases, the cells can include a heterologous DNA that encodes at least one enzyme that catalyzes the conversion of isobutyraldehyde to isobutyrate. In other cases, the cells can include a genetically modified enzyme that catalyzes the conversion of isobutyraldehyde to isobutyrate to a degree greater than the wild-type version of the enzyme. In other cases, the cells can include one or more enzyme that catalyze the conversion of 2-ketovaline to isobutyrate. Generally, methods include growing the cells in a medium that includes a carbon source that the cells are able to convert to isobutyrate.

### CROSS-REFERENCE TO RELATED APPLICATION

This application claims priority to U.S. Provisional Patent Application Ser. No. 61/441,939, filed Feb. 11, 2011.

### BACKGROUND

Isobutyric acid (also referred to herein as “isobutyrate”) is used in the production of fibers, resins, plastics, and dyestuffs, and is used as an intermediate in the manufacture of pharmaceuticals, cosmetics, and food additives. Isobutyrate also can be further converted to methacrylate (i.e., methacrylic acid—MAA) which is a commodity chemical.

MAA is often esterified to MMA (methyl methacrylate), a major commodity used in the production of plastics. MMA is often used to produce polymethyl methacrylate plastics, but also is used to produce, for example, ethylene methacrylate (EMA), butyl methacrylate (BMA), acrylic acid dope, adhesives, ion exchange resin, leather treatment chemicals, lubrication additives, and crosslinking agent. There are many routes to making MAA via traditional chemical synthesis techniques. Most routes begin with either natural gas or crude oil as the feedstock.

There is a need for new methods of producing commodity chemicals from renewable feedstocks. Producing commodity chemicals from renewable materials reduces the likelihood of economic impact from exhauting non-renewable feedstock materials and can spur economic development providing the renewable feedstocks.

### SUMMARY OF THE INVENTION

In one aspect, the invention provides a recombinant microbial cell modified to exhibit increased biosynthesis of isobutylc acid compared to a wild type control. In some cases, the recombinant microbial cell is a fungal cell such as, for example, a member of the Saccharomycetaceae family such as, for example, Saccharomyces cerevisiae. In other cases, the recombinant cell can be a bacterial cell such as, for example, a member of the phylum Protobacteria such as, for example, a member of the Enterobacteriaceae family (e.g., Escherichia coli) or a member of the Pseudomonaceae family (e.g., Pseudomonas putida). In other cases, the recombinant cell can be a bacterial cell such as, for example, a member of the phylum Firmicutes such as, for example, a member of the Bacillaceae family (e.g., Bacillus subtilis) or a member of the Streptococcaceae family (e.g., Lactococcus lactis).

In some embodiments, the recombinant microbial cell comprises at least one heterologous DNA molecule that encodes a polypeptide that catalyzes conversion of isobutyraldehyde to isobutyrate.

In some cases, the polypeptide that catalyzes conversion of isobutyraldehyde to isobutyrate comprises an aldehyde dehydrogenase such as, for example, E. coli phenylacetaldehyde dehydrogenase (PadA), E. coli acetaldehyde dehydrogenase (AldB), E. coli 3-hydroxypropionaldehyde dehydrogenase (AldH), E. coli succinate semialdehyde dehydrogenase (GabD), E. coli γ-aminobutyraldehyde dehydrogenase (YdcW), B. ambifaria α-ketoglutaric semialdehyde dehydrogenase (KDH_{ba}), or P. putida α-ketoglutaric semialdehyde dehydrogenase (KDHₚₚ).

In some embodiments, the polypeptide that catalyzes conversion of isobutyraldehyde to isobutyrate comprises a polypeptide having at least 80% amino acid sequence similarity to the amino acid sequence of any one of SEQ ID NO:1 through SEQ ID NO:106. In other embodiments, the polypeptide that catalyzes conversion of isobutyraldehyde to isobutyrate comprises a polypeptide having at least 80% amino acid sequence identity to the amino acid sequence of any one of SEQ ID NO:1 through SEQ ID NO:106.

In some embodiments, the heterologous DNA molecule comprises a DNA molecule that encodes a polypeptide having at least 80% amino acid sequence similarity to the amino acid sequence of any one of SEQ ID NO:1 through SEQ ID NO:106. In other embodiments, the heterologous DNA molecule comprises a DNA molecule that encodes a polypeptide having at least 80% amino acid sequence identitity to the amino acid sequence of any one of SEQ ID NO:1 through SEQ ID NO:106.

In still other embodiments, the recombinant cell can include at least one heterologous DNA molecule that encodes a polypeptide that is a member of a pathway that catalyzes conversion of 2-ketovaline to isobutyrate. In of these embodiments, the polypeptide that is a member of a pathway that catalyzes conversion of 2-ketovaline to isobutyrate comprises a branched-chain keto acid dehydrogenase. In some embodiments, the polypeptide that is a member of a pathway that catalyzes conversion of 2-ketovaline to isobutyrate comprises a thioesterase. In some of these embodiments, the thioesterase can include TesA or TesB.

In another aspect, the invention provides a genetically modified cell comprising at least one endogenous enzyme modified to increase its ability to convert isobutyraldehyde to isobutyrate. In some cases, the modified enzyme catalyzes the conversion of isobutyraldehyde to isobutyrate. In other cases, the modified enzyme increases the ability of the cell to tolerate an environment comprising a high level of isobutyrate.

In some embodiments of the recombinant microbial cell or genetically modified microbial cell, the cell further comprises a genetically modified version of a polypeptide that catalyzes the conversion of isobutyraldehyde to isobutanol, wherein the genetically modified version polypeptide exhibits a decrease in catalytic activity compared to the wild type polypeptide. In some cases, the genetically modified polypeptide comprises an alcohol dehydrogenase such as, for example, a polypeptide encoded by a genetically modified adhE or a genetically modified adhP. In other cases, the genetically modified polypeptide comprises an ethanolamine utilization protein such as, for example, a polypeptide encoded by a genetically modified eutG. In still other cases, the genetically modified polypeptide comprises a polypeptide encoded by a genetically modified yiaY, a genetically modified yqhD, or a genetically modified yigB.

In some embodiments of the recombinant microbial cell or genetically modified microbial cell, the cell further comprises a genetically modified version of a polypeptide that catalyzes the conversion of pyruvate to any one or more of lactate, formate, and acetate, wherein the genetically modified version polypeptide exhibits a decrease in catalytic activity compared to the wild type polypeptide. In some cases, the genetically modified polypeptide comprises a lactate dehydrogenase such as, for example, a polypeptide encoded by a genetically modified IdhA. In other cases, the genetically modified polypeptide comprises a pyruvate formate lyase I such as, for example, a polypeptide encoded by a genetically modified pflB. In other cases, the genetically modified polypeptide comprises a pyruvate oxidase such as, for example, a polypeptide encoded by a genetically modified poxB.

In some embodiments of the recombinant microbial cell or genetically modified microbial cell, the cell further comprises a genetically modified version of a polypeptide that catalyzes the conversion of acetyl-CoA to ethanol or acetyl-P, wherein the genetically modified version polypeptide exhibits a decrease in catalytic activity compared to the wild type polypeptide. In some cases, the genetically modified polypeptide comprises an alcohol dehydrogenase such as, for example, a polypeptide encoded by a genetically modified adhE. In other cases, the genetically modified polypeptide comprises a phosphate acetyltransferase such as, for example a polypeptide encoded by a genetically modified pta.

In some embodiments of the recombinant microbial cell or genetically modified microbial cell, the cell further comprises a polypeptide that catalyzes conversion of 2-ketoisovalerate to isobutyraldehyde such as, for example, a 2-ketoacid decarboxylase.

In some embodiments of the recombinant microbial cell or genetically modified microbial cell, the cell further comprises a plurality of polypeptides that sequentially catalyze conversion of pyruvate to 2-ketoisovalerate such as, for example, a dihydroxyacid dehydratase, a ketol-acid reductoisomerase, and an acetolactate synthase.

In another aspect, the invention provides a method that includes incubating a recombinant cell oe genetically modified cell as described herein in medium that comprises a carbon source under conditions effective for the cell to produce isobutyrate, wherein the carbon source comprises one or more of: glucose, Compound 6 of FIG. 1, Compound 7 of FIG. 1, Compound 8 of FIG. 1, Compound 9 of FIG. 1, and Compound 10 of FIG. 1. In some cases, the method further includes one or more steps converting isobutyrate to another compound.

In another aspect, the invention provides a method that includes introducing into a host cell a heterologous polynucleotide encoding a polypeptide that catalyzes conversion of isobutyraldehyde to isobutyrate operably linked to a promoter so that the modified host cell catalyzes conversion of isobutyraldehyde to isobutyrate.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. (a) Chemical synthesis of isobutyric acid from petrochemical feedstock and its representative applications. (b) Design of a metabolic pathway for biosynthesis of isobutyric acid from renewable carbon source glucose. Enzyme “X” efficiently converts isobutyraldehyde (10) into isobutyric acid (1).

FIG. 2. Biosynthesis of isobutyric acid with the synthetic metabolic pathway. (a) Construction of two synthetic operons for gene overexpression to drive the carbon flux towards isobutyric acid. (b) Production level with different aldehyde dehydrogenases: (i) no aldehyde dehydrogenase; (ii) aldB; (iii) aldH; (iv) gabD; (v) kdh_{ba}; (vi) kdhₚₚ; (vii) padA; (viii) ydcW.

FIG. 3. The effect of deleting competing pathway on biosynthesis. (a) Endogenous alcohol dehydrogenases such as YqhD compete with PadA for isobutyraldehyde and produce byproduct isobutanol. (b) The yqhD knockout greatly increases isobutyric acid production and decreases isobutanol level.

FIG. 4. Plasmid map of pIBA1.

FIG. 5. Plasmid map of pIBA3.

FIG. 6. Isobutyrate synthetic pathway in E. coli. Abbreviations: AlsS, acetolactate synthase; IlvC, 2,3-dihydroxy-isovalerate:NADP+oxidoreductase; IlvD, 2,3-dihydroxy-isovalerate dehydratase; KTVD, α-ketoisovalerate decarboxylase; PadA, phenylacetaldehyde dehydrogenase.

FIG. 7. Effect of alcohol dehydrogenase knockouts on isobutyrate fermentation in shake flask. (A) Isobutyrate production in different knockout strains. (B) Isobutanol formation in corresponding knockout strains. (i) IBA1-1C, ΔyqhD; (ii) IBA11-1C, ΔyqhDΔadhE; (iii) IBA12-1C, ΔyqhDΔadhP; (iv) IBA13-1C, ΔyqhDΔeutG; (v) BIA14-1C, ΔyqhDΔyiaY; (vi) IBA15-1C, ΔyqhDΔyjgB.

FIG. 8. Effect of PadA expression level on isobutyrate production in shake flask. (A) Isobutyrate level in different knockout strains with two copies of PadA. (B) Corresponding isobutanol formation. (i) IBA1-2C, ΔyqhD; (ii) IBA13-2C, ΔyqhDΔeutG; (iii) IBA14-2C, ΔyqhDΔyiaY; (iv) IBA15-2C, ΔyqhDΔyjgB.

FIG. 9. Scale-up fermentation of isobutyate by fed-batch culture in a bioreactor. (A) 50% NH₄OH; IBA15-2C strain. (B) 10N NaOH; IBA15-2C strain. (C) 20% Ca(OH)₂ suspension; IBA15-2C strain. (D) 20% Ca(OH)₂ suspension, IBA1-2C strain. Symbols: closed square, biomass; closed up triangle, acetate; open circle, isobutyrate.

FIG. 10. Isobutyrate synthetic pathway in E. coli. Abbreviations: AlsS, acetolactate synthase; IlvC, 2,3-dihydroxy-isovalerate:NADP+oxidoreductase; IlvD, 2,3-dihydroxy-isovalerate dehydratase; BKDH, branched-chain keto acid dehydrogenase; TesA, thioesterase A; TesB, thioesterase B.

FIG. 11. Plasmid map of pIBA16.

FIG. 12. Plasmid map of pIBA17.

FIG. 13. Plasmid map of pIBA18.

### DETAILED DESCRIPTION OF ILLUSTRATIVE EMBODIMENTS

Isobutyric acid is a platform chemical with many and varied applications. Current processes for manufacturing isobutyric acid involve the use of nonrenewable, unsustainable petroleum feedstocks and/or toxic materials. No natural organism can produce a commercially significant amount of isobutyric acid. We have, however, constructed recombinant cells that possess synthetic metabolic pathways for high-level biosynthesis of isobutyric acid from renewable feedstock such as, for example, glucose. Thus, we provide a novel route for synthesizing isobutyric acid that is not dependent on petroleum. We further provide novel recombinant microbes for synthesizing isobutyric acid.

As used in the description that follows, the term “and/or” means one or all of the listed elements or a combination of any two or more of the listed elements; the term “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Fossil-based resources are commonly exploited for energy and as chemical feedstocks. Due to the depletion of oil reserves, there is growing interest in exploring alternatives to petroleum-based products. Biosynthesis is a promising approach that enables sustainable production of certain fuels or certain chemicals from renewable carbon sources. (Atsumi et al., 2008 Nature 451:86-89; Steen et al., 2010 Nature 463:559-562; Causey et al., 2003 Proc. Natl. Acad. Sci. USA 100:825-832; Lin et al., 2005 Metab. Eng. 7:116-127; Zeng and Biebl, 2002 Adv. Biochem. Eng. Biotechnol. 2002:239-259; Alper et al., 2005 Nat. Biotechnol. 23:612-616). One challenge is that many useful chemicals are not naturally produced by biological systems. Therefore, it is often necessary to design or evolve novel metabolic pathways for the production of non-natural metabolites. (Zha et al., 2004 J. Am. Chem. Soc. 126:4534-4535; Zhang et al., 2008 Proc. Natl. Acad. Sci. USA 105:20653-20658; Yan et al., 2005 Appl. Environ. Microbiol. 71:3617-3623; Zhang et al., 2010 Proc. Natl. Acad. Sci. USA 107:6234-6239). Here we report the development of a biosynthetic route for producing isobutyric acid.

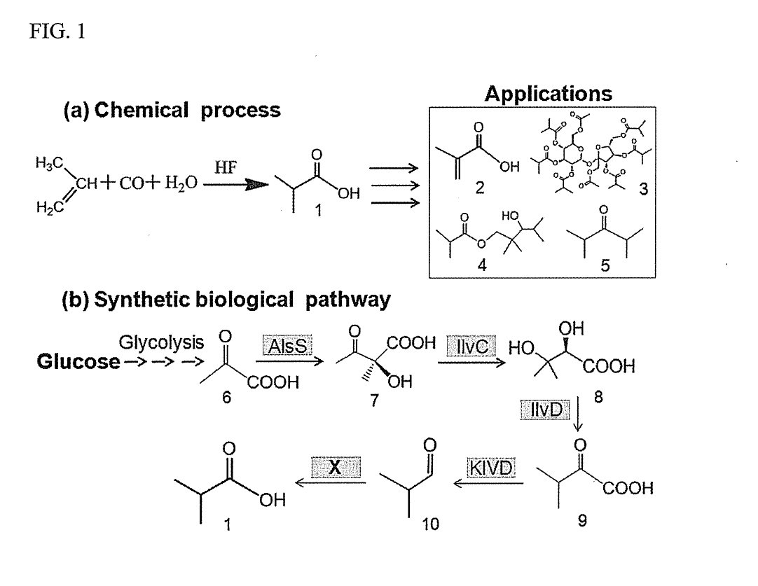

Isobutyric acid (FIG. 1(a), Compound 1) is a useful platform chemical. It can be converted to methacrylic acid (FIG. 1(a), Compound 2) by catalytic oxidative dehydrogenation. (Millet, 1998 Catal. Rev.-Sci. Eng. 40:1-38). Ester of methacrylic acid, methyl methacrylate, is produced in the quantity of 2.2 million tons per year for the synthesis of poly(methyl methacrylate). (Nagai, 2001 Appl. Catal. A-Gen. 221:367-377). Isobutyric acid also can be used to manufacture sucrose acetate isobutyrate (FIG. 1(a), Compound 3), an emulsifier that is used in printing inks, automotive paints, and beverage additives with a market size of 100,000 tons annually. (Godshall, “Sustainability of the Sugar and Sugar-Ethanol Industries,” ACS Symposium Series 1058; American Chemical Society: Washington, D.C., 2010; pp 253-268). Another application of isobutyric acid is for the synthesis of 2,2,4-trimethyl-1,3-pentanediol monoisobutyrate (FIG. 1(a), Compound 4; TEXANOL, Eastman Chemical Co., Kingsport, Tenn.) or diisobutyrate (TXIB). TXIB is a non-phthalate plasticizer and TEXANOL is a commonly used coalescent. (“Screening Information Data Set (SIDS) for High Production Volume Chemicals,” Organization for Economic Cooperation and Development 2005). Other exemplary applications of isobutyric acid include preparation of isopropyl ketones such as isobutyrone (FIG. 1(a), Compound 5) by decarboxylative coupling (see, e.g., U.S. Pat. No. 4,754,074).

One current manufacturing process of isobutyric acid involves an acid-catalyzed Koch carbonylation of propylene (FIG. 1(a); see, e.g., U.S. Pat. No. 4,452,999). This chemical process produces at least two concerns. First, propylene, the starting material, is produced by cracking larger hydrocarbon molecules that are most commonly derived from non-renewable resources such as petroleum and natural gas, whose long-term sustainable supply is not guaranteed. Second, the use of carbon monoxide and hydrogen fluoride may cause environmental damage. Such problems could be alleviated by replacing chemical synthesis with microbial biosynthesis.

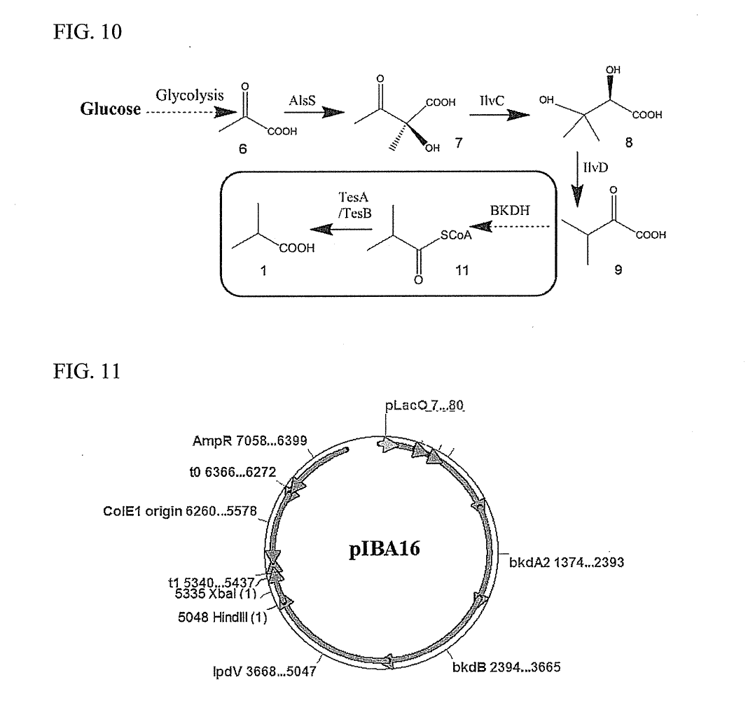

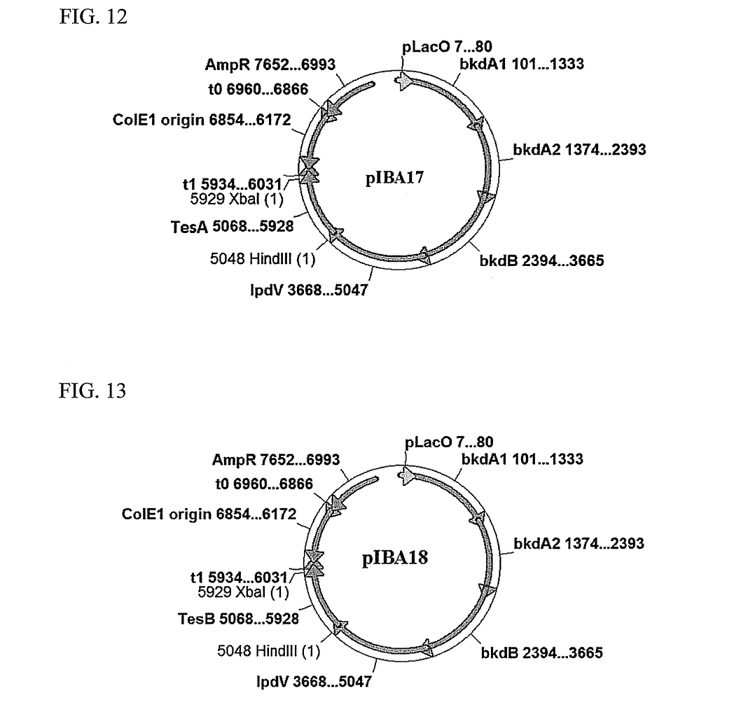

While there are some bacteria that can overproduce butyric acid (Liu et al., 2006 Enzyme Microb. Technol. 38:521-528), no natural organism is known to produce a commercially useful amount of isobutyric acid. We have developed a synthetic metabolic pathway that is based on the natural metabolic route for generating isobutyraldehyde from, for example, glucose. The natural metabolic pathway is augmented with at least one engineered metabolic step that diverts this natural metabolic pathway toward the production of isobutyrate (e.g., FIG. 1(b) and FIG. 10).

In one engineered pathway, shown in FIG. 1(b), glucose is metabolized to pyruvate (Compound 6) through glycolysis. Pyruvate is then converted into 2-ketovaline (Compound 9) by valine biosynthetic enzymes AlsS, IlvC, and IlvD. (Atsumi et al., 2008 Nature 451:86-89). 2-Ketovaline can be decarboxylated into isobutyraldehyde by Ehrlich pathway enzyme 2-ketoacid decarboxylase (KIVD) from Lactococcus lactis. (de la Plaza et al., 2004 FEMS Microbiol. Lett. 238:367-74). For this synthetic pathway, we needed to identify an enzyme, indicated in FIG. 1(b) as “X”, that could effectively catalyze the conversion of isobutyraldehyde into isobutyrate.

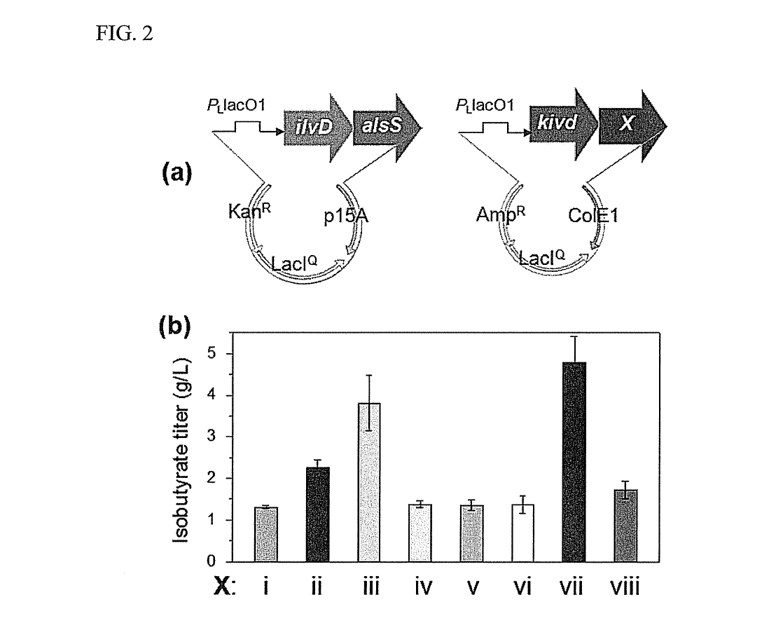

We identified enzymes capable of catalyzing the oxidation of isobutyraldehyde to isobutyrate even though isobutyraldehyde was a known natural or experimental substrate for none of the enzymes. We chose seven aldehyde dehydrogenases as possible candidate enzymes: E. coli acetaldehyde dehydrogenase AldB, E. coli 3-hydroxypropionaldehyde dehydrogenase AldH, E. coli succinate semialdehyde dehydrogenase GabD, E. coli phenylacetaldehyde dehydrogenase PadA, E. coli γ-aminobutyraldehyde dehydrogenase YdcW, Burkholderia ambifaria α-ketoglutaric semialdehyde dehydrogenase (KDH_{ba}), and Pseudomonas putida KT2440 α-ketoglutaric semialdehyde dehydrogenase (KDHₚₚ). These enzymes share little homology and cover a wide range of aldehyde substrates, although the wide range of aldehyde substrates does not include isobutyraldehyde. An oligonucleotide encoding one of the aldehyde dehydrogenases was cloned after KIVD to build an expression cassette kivd-x on a high copy plasmid (FIG. 2(a), with X representing the aldyhyde dehydrogenase-encoding oligonucleotide). Another operon on a medium copy plasmid in the transcriptional order ilvD-alsS (FIG. 2(a)) was also constructed to drive the carbon flux towards 2-ketovaline (ilvC was not cloned since the chromosomal copy could be overexpressed upon induction by its substrate acetolactate; Wek and Hatfield, 1988 Mol. Biol. 203:643-663). This was repeated for each of the aldehyde dehydrogenases, resulting in a library of expression cassettes, each expressing one of the aldehyde dehydrogenases.

The cloned plasmids were transformed into wild type E. coli strain BW25113. Shake flask fermentation was performed at 30° C. for 48 hours. Cultures were grown in M9 minimal medium containing 40 g/L glucose as carbon source, and 0.1 mM IPTG was added to induce protein expression. Fermentation products were quantified by HPLC analysis with refractive index detection. As can be seen from FIG. 2(b), the aldehyde dehydrogenases provided varying levels of isobutyrate production. Without any plasmid-encoded aldehyde dehydrogenase, 1.3 g/L isobutyrate was detected (i, FIG. 2(b)), which should come from the function of endogenous aldehyde dehydrogenases. GabD, Kdh_{ba}, Kdhₚₚ and YdcW slightly increased the production level of isobutyrate (FIG. 2(b) iv, v, vi, and viii, respectively). In comparison, transformants possessing AldB and AldH produced 2.3 g/L and 3.8 g/L isobutyrate (FIG. 2(b) ii, iii, respectively). Transformants possessing PadA produced 4.8 g/L isobutyrate (FIG. 2(b), vii).

Because it produced the greatest amount of isobutyrate, we selected PadA for further study. To characterize the enzyme, PadA was tagged with an N-terminal 6×His-tag, overexpressed, and purified through a Ni-NTA column. The kinetic parameters for conversion of isobutyraldehyde by PadA were determined by measuring the reduction of NAD+ to NADH at 340 nm. Results are shown in Table 1. PadA activity toward isobutyraldehyde is much lower than that toward its natural physiological substrate phenylacetaldehyde. Though the k_{cat} value is only 4-fold lower (1494 min⁻¹ versus 5810 min⁻¹), the Kₘ value is 230-fold higher (2.67 mM versus 0.0116 mM). Thus the specificity constant k_{cat}/Kₘ of PadA towards phenylacetaldehyde is almost 1000-fold higher than towards the non-natural substrate isobutyraldehyde.

TABLE 1
Kinetic parameters of PadA.

| | | | k_{cat}/Kₘ |
|---|---|---|---|
| Substrate | Kₘ (mM) | k_{cat} (min⁻¹) | (mM⁻¹ · min⁻¹) |
| Isobutyraldehyde | 2.67 ± 0.17 | 1494 ± 30 | 560 |
| Phenylacetaldehyde* | 0.0116 | 5810 | 501,000 |

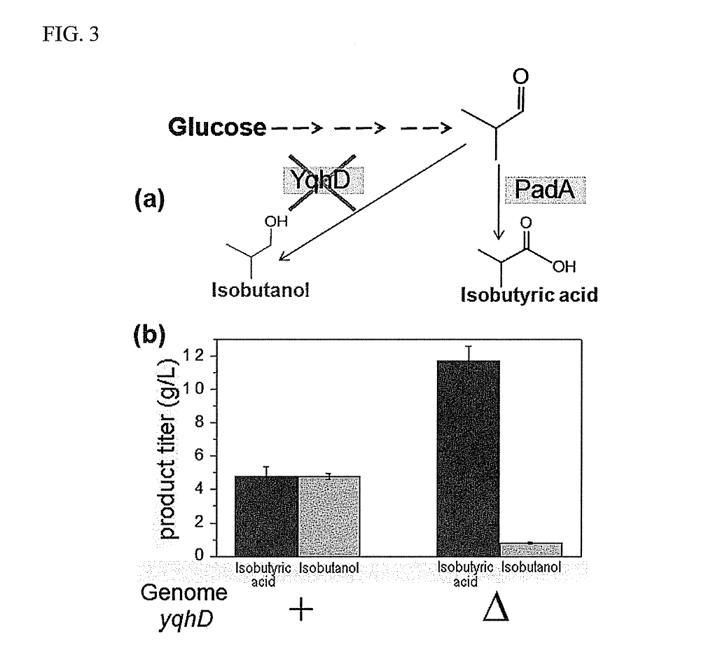

Since PadA has a relatively high Kₘ for isobutyraldehyde, endogenous alcohol dehydrogenases such as YqhD (Kₘ 1.8 mM; Atsumi et al., 2010 Appl. Microbiol. Biotechnol. 85:651-657) can compete for the aldehyde substrate and produce isobutanol rather than isobutryric acid (FIG. 3(a)). This may explain, in part, the accumulation of 4.8 g/L isobutanol in the fermentation product, equal to the concentration of isobutyrate (FIG. 3(b)). We deleted the yqhD gene from the chromosome of BW25113. Compared to the wild type strain, the ΔyqhD mutant decreased the isobutanol production to 0.8 g/L and increased the isobutyrate production to 11.7 g/L (FIG. 3(b)). Thus, in shake flask fermentation this modified strain can produce isobutyrate with a yield of 0.29 g/g glucose (FIG. 3(b)) which is 59% of the theoretical maximum.

Thus, we have developed a synthetic metabolic pathway for biosynthesis of isobutyrate from glucose. We discovered that each of the seven aldehyde dehydrogenases we investigated converted isobutyraldehyde to isobutyrate. Of these seven aldehyde dehydrogenases, PadA was the most effective enzyme in oxidizing isobutyraldehyde to isobutyrate in vivo. Deleting from chromosome the yqhD gene, which encodes an enzyme that competes with PadA for isobutyraldehyde, further increased isobutyrate production to 11.7 g/L from 40 g/L glucose.

In an alternate engineered, pathway, shown in FIG. 10, 2-ketovaline (Compound 9) is converted to isobutyrate (Compound 1). The branched-chain keto acid dehydrogenase BKDH can convert 2-ketovaline to a branched-chain CoA, which can, in turn, be converted to isobutyrate by a thioesterase.

We cloned bkdh from Pseudomonas putida genomic DNA, and tesA and tesB from wild type E. coli genomic DNA. Plasmid pIBA16 contains bkdh, pIBA17 contains bkdh and TesA, and pIBA18 contains bkdh and TesB. The construct that includes BKDH without TesB or TesA (pIBA16) accumulated isobutyrate to a concentration of 5.61±0.67 g/L (Table 4), somewhat higher than the isobutyrate production exhibited by the PadA construct prior to knocking out of yqhD (FIG. 3(a)). The addition of a thioesterase, however, further increased the isobutyrate yield. For example, BKDH plus TesB (pIBA18) produced 8.6 g/L isobutyrate from 40 g/L glucose (0.22 g/g glucose, or about 44% of the theoretical maximum yield).

Consequently, we have demonstrated various modified metabolic routes to achieve isobutyrate biosynthesis. Moreover, we have demonstrated that one can achieve efficient isobutyrate biosynthesis by diverting biosynthesis from various points along an endogenous biosynthetic pathway that does not natively produce isobutyrate. We have, therefore, established a general platform for biosynthesis of isobutyrate.

### Effect of Knockouts on Isobutyrate Production

We next built on our initial findings by investigating whether further engineering could further increase carbon yield. We constructed six E. coli knockout strains and found that one double knockout (ΔyqhD, ΔyjgB) produced 17% more isobutyrate than the single knockout strain (ΔyqhD). We then introduced an additional copy of aldehyde dehydrogenase under a constitutive promoter on a plasmid. PadA overexpression further reduced isobutanol formation and further increased isobutyrate production. Thus we were successful in constructing an engineered strain that has an isobutyate yield of 0.39 g/g glucose, 80% of the theoretical maximum.

We also scaled up the fermentation process from shake flasks to a bioreactor. We found that Ca(OH)₂ was much better than NH₄OH or NaOH as the base for pH adjustment during fetmentation. The use of Ca(OH)₂ to maintain the pH of the fermentation culture increases cell density, decreases acetate accumulation, and increased the final accumulation of isobutyrate to 90 g/L.

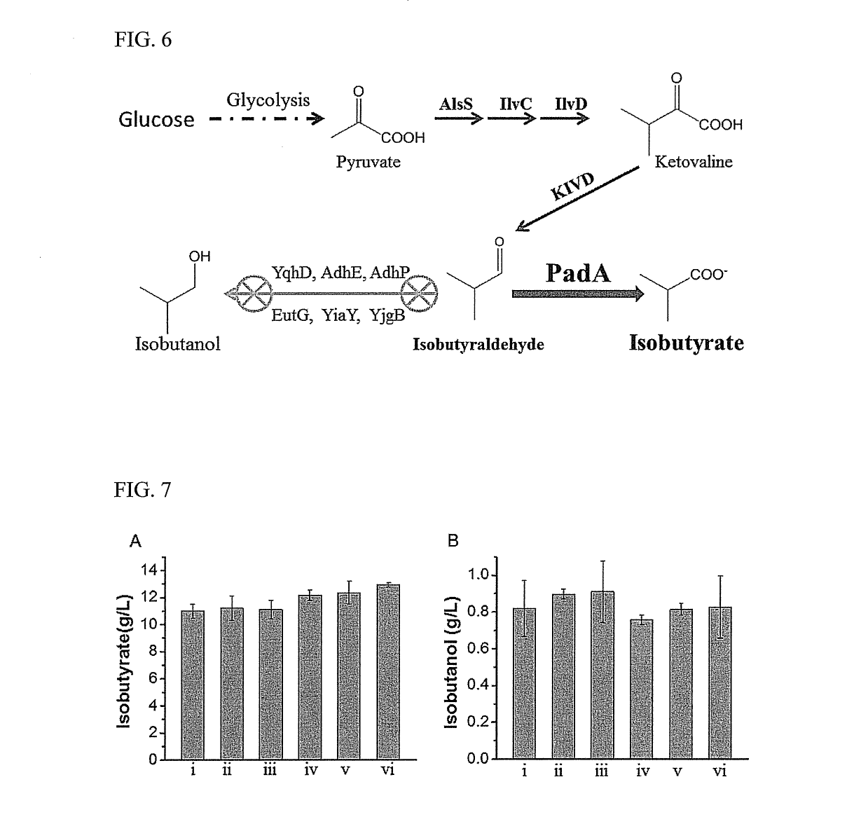

In the engineered biosynthetic pathway illustrated in FIG. 1(b) and FIG. 6, isobutyraldehyde is the immediate precursor to isobutyrate. In many organisms, isobutyraldehyde is naturally reduced to isobutanol by an endogenous alcohol dehydrogenase such as, for example, AdhE, AdhP, EutG, YiaY, YjgB and YqhD in E. coli. YqhD is known to be involved in isobutanol formation since yqhD knockouts can exhibit a 50% increase in isobutyrate production. (Zhang et al., 2011 ChemSusChem 4:1068-1070). However, even after yqhD knockout, isobutanol was still present as a fermentation byproduct with a concentration of 0.8 g/L (FIG. 7B, i). Therefore, we investigated whether knockouts of other alcohol dehydrogenase genes—in combination with a deletion of yqhD—can decrease conversion of isobutyraldehyde to isobutanol and thus increase the amount of isobutyraldehyde available to the cell for conversion to isobutyrate. The additional deletion of adhE or adhP slightly increased isobutanol accumulation to 0.90 g/L (FIG. 7B, ii and iii, respectively), while isobutyrate production was not affected (FIG. 7A, ii and iii, respectively). Knocking out eutG decreased isobutanol level to 0.76 g/L and increased isobutyrate concentration to 12.2 g/L (FIG. 7B, iv and FIG. 7A, iv, respectively). Interestingly, while the additional deletion of either yiaY or yjgB did not reduce isobutanol formation (FIG. 7B, v and vi, respectively), they increased isobutyrate production level to 12.4 g/L and 12.9 g/L (FIG. 7A, v and vi, respectively). Thus, compared to IBA1-1C strain (ΔyqhD, i), IBA15-1C strain (ΔyqhD, ΔyjgB, vi) exhibited an increase in isobutyrate production. The results in ΔadhE strain were different from a recently published report (Trinh et al., 2011 Appl. Environ. Microbiol. 77:4894-4904). However, that study used anaerobic condition to investigate the function of adhE for isobutanol production, while our fermentation condition was semianaerobic. AdhE enzyme is known to be inactivated by oxygen (Holland-Staley et al., 2000 J. Bacteriol. 182:6049).

### Effect of PadA Expression Level on Isobutyrate Production

Next we examined an alternative approach to directing conversion of isobutyraldehyde to isobutyrate that involved increasing the protein expression level of PadA to be more competitive against alcohol dehydrogenases that convert isobutyraldehyde to isobutanol. An additional copy of padA was introduced under a constitutive promoter on a high copy plasmid. We added the second copy of padA to the single knockout strain IBA1 (ΔyqhD), and to double knockout strains IBA13 (ΔyqhDΔeutG), IBA14 (ΔyqhDΔyiaY), and IBA15 (ΔyqhDΔyjgB), each of which produced more isobutyrate than the single knockout IBA1 strain when carrying one copy of padA (FIG. 7A).

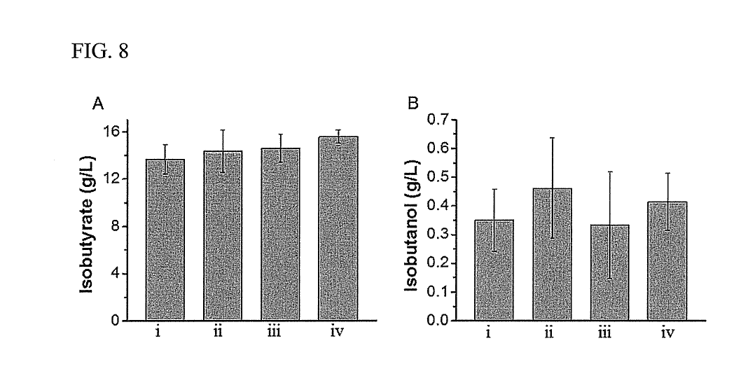

The double-PadA strain IBA1-2C produced 13.7 g/L isobutyrate (FIG. 8A, i) as compared to 11 g/L from the single-PadA parental strain, IBA1-1C (FIG. 7A, i). On the other hand, isobutanol concentration was reduced to 0.35 g/L (FIG. 8B, i) from 0.82 g/L (FIG. 7B, i). These results demonstrate that increasing expression of PadA decreases isobutanol accumulation and increases isobutyrate production. The isobutanol decrease (0.47 g/L) was less than the isobutyrate increase (2.7 g/L). One possible reason for the disparity in the difference may be that producing isobutyrate is less stressful on the cells than producing isobutanol so that the cells containing two copies of padA produced smaller amounts of byproducts and therefore direct more biosynthetic energy toward production of isobutyrate. For example, accumulation of acetate, another byproduct, also was reduced: from 0.6 g/L in IBA1-1C to 0.1 g/L in IBA1-2C.

The effect of PadA overexpression was confirmed in IBA13-2C, IBA14-2C, and IBA15-2C strains as well. With two copies of padA, these strains generated around 0.4 g/L isobutanol (FIG. 8B, ii-iv), significantly lower than the strains carrying one copy of PadA (FIG. 7B, iv-vi). More importantly, IBA13-2C, IBA14-2C, and IBA15-2C also increased accumulation of isobutyrate (14.3 g/L, 14.6 g/L, and 15.6 g/L (FIG. 8A, ii-iv), respectively) compared to their respective single-PadA parental strains (FIG. 7A, iv-yl).

Moreover, isobutyrate accumulation in the PadA overexpressing double knockouts IBA-13-2C, IBA14-2C, and IBA15-2C were higher than the isobutyrate accumulation in the PadA overexpressing single (Ayqh) knockout in IBA1-2C, confirming that double knockouts increase isobutyrate production.

Thus, one can engineer microbes to favor production of isobutyrate rather than isobutanol by overexpressing PadA, which favors conversion of isobutyraldehyde to isobutyrate, and/or knocking out one or more aldehyde dehydrogenases that can compete with PadA for isobutyraldehyde but favor conversion of isobutyraldehyde to isobutanol. The PadA overexpressing double knockout (Δyqh, ΔyjgB) strain IBA15-2C yielded 0.39 g/g glucose, 80% of the theoretical maximum.

### Optimize the Fermentation Conditions in a Fed-Batch Bioreactor

We next investigated whether the effects described above are maintained when fermentation is scaled up from a shake flask to a bioreactor. We performed bioreactor fermentation experiments with the strain IBA15-2C. To avoid overaccumulation of acetate in the bioreactor, the glucose feeding rate was adjusted to keep glucose at a level below 10 g/L. Since two molecules of NADH are generated for each molecule of isobutyrate produced, the dissolved oxygen (DO) level was maintained at 10% to burn excess NADH. Higher DO levels were avoided in order to prevent excessive oxidation of substrate into CO₂ through the TCA cycle.

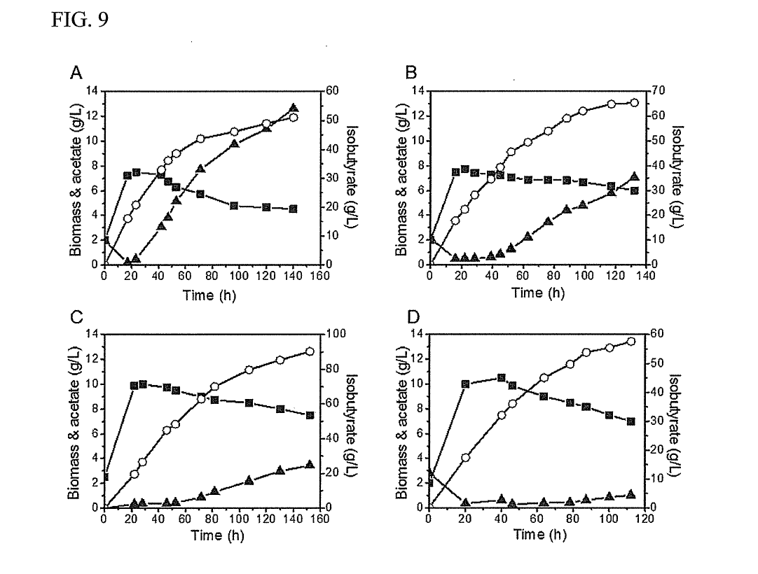

During biosynthesis of isobutyrate, pH can drop sharply if base is not added to the fermentation culture medium. We investigated the effect of three different bases, NH₄OH, NaOH, and Ca(OH)₂ to maintain a pH of 7.0. As seen in FIG. 9A-C (closed square), for all conditions, the biomass increased exponentially at the first 20 hours, and then decreased gradually. The maximum biomass obtained using either NH₄OH or NaOH was about 7.5 g/L, whereas the maximum biomass obtained Ca(OH)₂ was about 10 g/L. This result suggests that excessive ammonium ion or sodium ion might have a negative impact on cell growth. Ammonium hydroxide has previously been used to control pH and provide a supply a source of nitrogen, but this base apparently is less than optimal for maximizing isobutyrate production. Isobutyrate accumulation reached 51.1 g/L after 140 hours using NH₄OH (FIG. 9A, open circle), 65.4 g/L with NaOH (FIG. 9B, open circle), and 90.3 g/L with Ca(OH)₂ (FIG. 9C, open circle). Generally, the final accumulation of isobutyrate was inversely related to the final accumulation of acetate in each culture: the NH₄OH-adjusted culture accumulated 12.6 g/L acetate, while acetate decreased to 7.1 g/L in the NaOH-adjusted culture and only 3.4 g/L in the Ca(OH)₂-adjusted culture (FIG. 9A-C, closed triangle). This is consistent with previous reports that acetate was a major inhibitor of E. coli fermentation (Eiteman and Altman, 2006 Trends Biotechnol. 24:530-536; Koh et al., 1992 Biotechnol. Lett. 14:1115-1118). In summary, using Ca(OH)₂ to maintain a culture pH of 7.0 increased cell density, increased isobutyrate accumulation and decreased acetate byproduct compared to the use of NH₄OH or NaOH.

As a control, the fermentation of the PadA overexpressing single gene (yqhD) knockout strain IBA1-2C in a bioreactor was also investigated. This strain produced 57.6 g/L isobutyrate and 1.0 g/L acetate after 122 hours (FIG. 9D), confirming that calcium hydroxide helped decrease acetate formation and increase isobutyrate production. However, IBA15-2C strain produced 57% more isobutyrate than IBA1-2C strain under the same conditions, which suggests that the increased isobutyrate production observed shake flask cultures of the ΔyqhD/ΔygjB double knockout can be scaled up to bioreactor volumes.

This work demonstrates that isobutyrate can be produced from engineered microbes with a high accumulation and high yield. Since the production of isobutyrate described in this work is amenable to microbial fermentation, the modified microbial strains and the methods described herein can provide a new platform for commercial production of isobutyrate.

We have, therefore, developed a platform for producing isobutyrate in a renewable fashion. We have addressed problems associated with chemical synthesis such as the use of unsustainable petroleum feedstocks and toxic materials. Our biosynthetic approach provides an attractive option for the benefit of both economy and environment (Dale, 2003 J. Chem. Technol. Biotechnol. 78:1093-1103).

Thus, in one aspect, the invention provides a recombinant microbial cell modified to exhibit increased biosynthesis of isobutylc acid compared to a wild type control. As used herein, “increased production” can be characterized as a relative increase in biosynthesis of isobutyrate compared to a wild type control, as biosynthesis sufficient for a culture of the microbial cell to accumulate isobutyrate to a predetermined concentration, as an increase in the ratio of isobutyrate:isobutanol produced by the cell, or as an increase in the percentage of maximum theoretical yield using a specified reference feedstock such as, e.g., glucose.

Specifying a reference feedstock such as glucose does not require that the microbial culture be grown using the specified reference feedstock as a carbon source or energy source. Indeed, as described in more detail below, feedstock can include, for example, any of Compounds 6-9 shown in FIG. 1(b). Those of ordinary skill in the art, however, are able to arithmetically convert a theoretical maximum yield using any alternative feedstock to a corresponding theoretical maximum yield based on a metabolically equivalent amount of any reference feedstock.

Thus, in some cases, a modified microbial cell can exhibit an increase in biosynthsis of isobutyrate that reflects at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the isobutyrate produced by an appropriate wild type control, up to and including the fold increase necessary for a given host cell to produce isobutyrate at the theoretical maximum of 0.49 g isobutyrate/g glucose.

In other cases, a modified microbial cell can exhibit an increase in the biosynthesis of isobutyrate reflected by accumulation of isobutyrate to a predetermined concentration when the microbial cell is grown for a specified time in culture. The predetermined concentration may be any predetermined concentration of isobutyrate suitable for a given application. Thus, a predetermined concentration may be, for example, a concentration of at least 0.1 g/L such as, for example, at least 0.5 g/L, at least 1.0 g/L, at least 2.0 g/L, at least 3.0 g/L, at least 4.0 g/L, at least 5.0 g/L, at least 6.0 g/L, at least 7.0 g/L, at least 8.0 g/L, at least 9.0 g/L, at least 10 g/L, at least 20 g/L, at least 50 g/L, at least 55 g/L, at least 60 g/L, at least 65 g/L, at least 70 g/L, at least 75 g/L, at least 80 g/L, at least 85 g/L, at least 90 g/L, at least 95 g/L, at least 100 g/L, at least 110 g/L, at least 120 g/L, at least 130 g/L, at least 140 g/L, at least 150 g/L, at least 160 g/L, at least 170 g/L, at least 180 g/L, at least 190 g/L, or at least 200 g/L.

In batch culture, the specified time may have a minimum of at least 12 hours such as, for example, at least 24 hours, at least 36 hours, at least 48 hours, at least 60 hours, at least 72 hours, at least 84 hours, at least 96 hours, at least 108 hours, at least 120 hours, at least 132 hours, or at least 144 hours. In batch culture, the specified time may have a miximum of no more than 240 hours such as no more than 216 hours, no more than 192 hours, no more than 168 hours, no more than 144 hours, no more than 120 hours, no more than 108 hours, no more than 96 hours, no more than 84 hours, no more than 72 hours, no more than 60 hours, or no more than 48 hours. In batch culture, the specified time also may be expressed as a range with endpoints defined by any minimum time and any appropriate maximum time. In continuous culture, the specified time may be expressed as an absolute amount of time in the same way as for a batch culture. Alternatively, the specified time in continuous culture may be expressed in terms of a stage of the culture such as, for example, homeostasis.

In certain embodiments, therefore, a modified cell can exhibit an increase in the biosynthesis of isobutyrate that can be characterized as producing at least 4.7 g/L isobutyrate after 48 hours. In other exemplary embodiments, the increase in isobutyrate production may be expressed in terms of accumulating at least 90 g/L isobutyrate after 120 hours of culture.

In other cases, a modified microbial cell can exhibit an increase in biosynthesis of isobutyrate that is characterized in terms of the ratio of isobutyrate:isobutanol produced by the cell. An increase in the biosynthesis of isobutyrate can be expressed as an isobutyrate:isobutanol ratio of at least 1:1 such as, for example, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 20:1, at least 25:1, at least 30:1, at least 50:1, at least 60:1, at least 70:1, at least 80:1, at least 90:1, or at least 100:1.

In still other cases, a modified microbial cell can exhibit an increase in biosynthesis of isobutyrate that reflects a predetermined isobutyrate yield of at least 40% of the theoretical yield from a specified reference feedstock such as, for example, glucose. The predetermined isobutyrate yield can be, for example, at least 40% of the theoretical maximum yield, at least 50% of the theoretical maximum yield, at least 60% of the theoretical maximum yield, at least 70% of the theoretical maximum yield, at least 80% of the theoretical maximum yield, at least 90% of the theoretical maximum yield, at least 95% of the theoretical maximum yield, at least 96% of the theoretical maximum yield, at least 97% of the theoretical maximum yield, at least 98% of the theoretical maximum yield, or at least 99% of the theoretical maximum yield. Certain embodiments can produce isobutyrate at about 44%, about 59%, or about 80% of the theoretical maximum yield from glucose.

The recombinant cell can be, or be derived from, any suitable microbe including, for example, a prokaryotic microbe or a eukaryotic microbe. In some embodiments, the cell can include at least one heterologous DNA molecule. As used herein, the term “or derived from” in connection with a microbe simply allows for the “host cell” to possess one or more genetic modifications before being modified to include a heterologous DNA molecule that encodes a polypeptide that is involved in an engineered biosynthetic pathways that results in biosynthesis of isobutyrate. Thus, the term “recombinant cell” encompasses a “host cell” that may contain nucleic acid material from more than one species before having the heterologous DNA molecule that encodes a polypeptide that is involved in, for example, either the conversion of isobutyraldehyde to isobutyrate or the conversion of 2-ketovaline to isobutyrate introduced into the cell.

In some embodiments, the recombinant cell may be, or be derived from, a eukaryotic microbe such as, for example, a fungal cell. In some of these embodiments, the fungal cell may be, or be derived from, a member of the Saccharomycetaceae family such as, for example, Saccharomyces cerevisiae, a member of the genus Candida such as, for example, Candida albicans, a member of the genus Kluyvermyces, or a member of the genus Pichia such as, for example, Pichia pastoris. In other embodiments, the fungal cell may be a member of the family Dipodascaceae such as, for example, Yarrowia lipolytica.

In other embodiments, the recombinant cell may be, or be derived from, a prokaryotic microbe such as, for example, a bacterium. In some of these embodiments, the bacterium may be a member of the phylum Protobacteria. Exemplary members of the phylum Protobacteria include, for example, members of the Enterobacteriaceae family (e.g., Escherichia coli) and, for example, members of the Pseudomonaceae family (e.g., Pseudomonas putida). In other cases, the bacterium may be a member of the phylum Firmicutes. Exemplary members of the phylum Firmicutes include, for example, members of the Bacillaceae family (e.g., Bacillus subtilis) and, for example, members of the Streptococcaceae family (e.g., Lactococcus lactis).

In the description that follows, descriptions of various embodiments refer to a heterologous DNA molecule that encodes a genetic modification. Combinations of the various embodiments are also possible. In such embodiments, more than one genetic modification can be included on a single heterologous DNA molecule such as, for example, a plasmid vector. Alternatively, different genetic modifications may be included on different vactors, each opf which is introduced into the host cell.

In some embodiments, the cell can include at least one heterologous DNA molecule that encodes a polypeptide that catalyzes conversion of isobutyraldehyde to isobutyrate. In some embodiments, the polypeptide that catalyzes conversion of isobutyraldehyde to isobutyrate comprises an aldehyde dehydrogenase. As used herein, the term “aldehyde dehydrogenase” refers to a polypeptide that, regardless of its common name or native function, catalyzes the conversion of isobutyraldehyde to isobutyrate. Exemplary aldehyde dehydrogenases include, for example, E. coli phenylacetaldehyde dehydrogenase (P adA), E. coli acetaldehyde dehydrogenase (AldB), E. coli 3-hydroxypropionaldehyde dehydrogenase (AldH), E. coli succinate semialdehyde dehydrogenase (GabD), E. coli γ-aminobutyraldehyde dehydrogenase (YdcW), B. ambifaria α-ketoglutaric semialdehyde dehydrogenase (KDH_{ba}), or P. putida α-ketoglutaric semialdehyde dehydrogenase (KDHₚₚ). In certain embodiments, the recombinant cell can include a heterologous DNA molecule—or a plurality of heterologous DNA molecules—that encodes a combination of two or more aldehyde dehydrogenases.

In other embodiments, the polypeptide encoded by the heterologous DNA molecule (i.e., the heterologously-encoded polypeptide) that catalyzes conversion of isobutyraldehyde to isobutyrate comprises, or is structurally similar to, a reference polypeptide that comprises the amino acid sequence of one or more of SEQ ID NO:1 through SEQ ID NO:106.

As used herein, a heterologously-encoded polypeptide is “structurally similar” to a reference polypeptide if the amino acid sequence of the heterologously-encoded polypeptide possesses a specified amount of similarity and/or identity compared to the reference polypeptide. Structural similarity of two polypeptides can be determined by aligning the residues of the two polypeptides (for example, a heterologously-encoded polypeptide and the polypeptide of, for example, any one of SEQ ID NO:1 through SEQ ID NO:106) to optimize the number of identical amino acids along the lengths of their sequences; gaps in either or both sequences are permitted in making the alignment in order to optimize the number of identical amino acids, although the amino acids in each sequence must nonetheless remain in their proper order.

A pair-wise comparison analysis of amino acid sequences can be carried out using the BESTFIT algorithm in the GCG package (version 10.2, Madison, Wis.). Alternatively, polypeptides may be compared using the Blastp program of the BLAST 2 search algorithm, as described by Tatiana et al., (1999 FEMS Microbiol Lett, 174:247-250), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide of the invention may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, biologically active analogs of a polypeptide containing deletions or additions of one or more contiguous or noncontiguous amino acids that do not eliminate a functional activity of the polypeptide are also contemplated.

A heterologously-encoded polypeptide can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to the reference amino acid sequence.

A heterologously-encoded polypeptide can include a polypeptide with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

Exemplary reference amino acid sequences include the amino acid sequence of any one of SEQ ID NO:1 through SEQ ID NO:106.

1WNB is the crystal structure of E. coli protein YdcW complexed with NADH and betaine aldehyde (Gruez et al. 2004 J. Mol. Biol. 343:29-41). Based on the crystal structure, residues Y150, D279, F436, and L438 are within a radius of 5 Å of the α-carbon of betaine aldehyde substrate. While the homology between PadA and YdcW is low, the binding pocket is well conserved. The corresponding residues in the active site of PadA are F175, V305, T461, and 1463. Similar analyses may be performed to identify amino acids residues that may be modified without interfering with the catalytic activity of the polypeptide and, just as important, to identify amino acid residues that are likely to be involved in substrate binding and/or catalytic activity.

In some embodiments, the recombinant cell can include a heterologous DNA molecule that encodes a polypeptide having at least 80% amino acid sequence similarity to the amino acid sequence of any one of SEQ ID NO:1 through SEQ ID NO:106. Thus, exemplary heterologous DNA molecules include those that encode a polypeptide having, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to the reference amino acid sequence.

In other embodiments, the heterologous DNA molecule encodes a polypeptide having at least 80% amino acid sequence identity to the amino acid sequence of any one of SEQ ID NO:1 through SEQ ID NO:106. Thus, exemplary heterologous DNA molecules include those that encode a polypeptide having, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

A heterologously-encoded polypeptide can further be designed to provide additional sequences, such as, for example, the addition of coding sequences for added C-terminal or N-terminal amino acids that would facilitate expression or purification by trapping on columns or use of antibodies. Such tags include, for example, histidine-rich tags that allow purification of polypeptides on nickel columns. Such gene modification techniques and suitable additional sequences are well known in the molecular biology arts.

In other embodiments, a recombinant cell can include at least one heterologous DNA molecule that encodes a polypeptide that catalyzes conversion of 2-ketovaline to isobutyrate.

In some embodiments, the polypeptide that catalyzes conversion of isobutyraldehyde to isobutyrate comprises a branched-chain keto acid dehydrogenase (BKDH). As used herein, the term “branched-chain keto acid dehydrogenase” refers to a polypeptide that, regardless of its common name or native function, catalyzes the conversion of 2-ketovaline to a branched-chain CoA. Exemplary branched-chain keto acid dehydrogenases can include, for example, BKDH of Pseudomonas putida. In some of these embodiments, the recombinant cell can include at least one heterologous DNA that encodes a thioeserase. As used herein, the term “thioesterase” refers to a polypeptide that, regardless of its common name or native function, catalyzes the conversion of a a branched-chain CoA to isobutyrate. Exemplary thioesterases can include, for example, TesA or TesB of E. coli.

In some embodiments, a recombinant cell can further include one or more polypeptides that catalyze a biosynthetic conversion illustrated in FIG. 1(b). Thus, for example, the recombinant cell can further include a polypeptide that catalyzes conversion of 2-ketoisovalerate to isobutyraldehyde such as, for example, 2-ketoacid decarboxylase; or, for example, any one or more of the polypeptides that catalyze a step in the conversion of pyruvate to 2-ketoisovalerate such as, for example, one or more of: a dihydroxyacid dehydratase, a ketol-acid reductoisomerase, and an acetolactate synthase.

In another aspect, the invention provides a genetically modified cell in which at least one endogenous enzyme is modified to increase its ability to convert isobutyraldehyde to isobutyrate. In some embodiments, the genetically modified cell can include one or mutations to one or more endogenous enzymes. In other embodiments, the genetically modified cell can include one or more mutations to one or more polypeptides that increase the ability of the cells to tolerate high levels of isobutyrate in culture. The mutations may be produced using molecular biology techniques including, for example, one or more of: transcriptome analysis, genome sequencing, cloning, site-specific mutagenesis, and transformation of the microbe with a vector that includes a polynucleotide that encodes the modified enzyme or enzymes. Alternatively, the mutations may be produced using classical microbial genetic techniques such as, for example, growth in or on a medium designed to select and/or identify microbes possessing desired spontaneous mutations.

In some embodiments, the recombinant cell or genetically modified cell can further include a genetically modified version of a polypeptide that catalyzes the conversion of isobutyraldehyde to a product other than isobutyrate such as, for example, isobutanol, lactate, ethanol, or acetyl-P, and thereby directs more isobutyraldehyde toward the biosynthesis of isobutyrate. Generally, the genetically modified version polypeptide can exhibit reduced catalytic activity compared to the wild type polypeptide. Such a genetic modification can decrease the extent to which isobutyraldehyde is metabolized in a manner that results in biosynthesis of products other than isobutyrate such as, for example, isobutanol, lactate, ethanol, or acetyl-P, and thereby increase the extent to which isobutyraldehyde is converted to isobutyrate.

In some cases, the recombinant cell or genetically modified cell can include a genetically modified version of a polypeptide that catalyzes the conversion of isobutyraldehyde to isobutanol. An exemplary polypeptide of this type can include, for example, can be a genetically modified version of an alcohol dehydrogenase such as, for example, a polypeptide encoded by a genetically modified adhE or a genetically modified adhP. In other embodiments, the genetically modified polypeptide can be genetically modified version of an ethanolamine utilization protein such as, for example, a polypeptide encoded by a genetically modified eutG. In some embodiments, the genetically modified polypeptide can be a polypeptide encoded by a genetically modified dkgA, genetically modified yiaY, a genetically modified yqhD, or a genetically modified yjgB

In some cases, the recombinant cell or genetically modified cell can include a genetically modified version of a polypeptide that catalyzes the conversion of pyruvate to any one or more of lactate, formate, and acetate, wherein the genetically modified version polypeptide exhibits a decrease in catalytic activity compared to the wild type polypeptide. An exemplary polypeptide of this type can include, for example, a genetically modified version of a lactate dehydrogenase such as, for example, a polypeptide encoded by a genetically modified idhA; a genetically modified version of a pyruvate formate lyase I such as, for example, a polypeptide endcoded by a genetically modified pflB; or a genetically modified version of a pyruvate oxidase such as, for example, a polypeptide encoded by a genetically modified poxB.

In some case, the recombinant cell or genetically modified cell can include a genetically modified version of a polypeptide that catalyzes the conversion of acetyl-CoA to ethanol or acetyl-P, wherein the genetically modified version of the polypeptide exhibits a decrease in catalytic activity compared to the wild type polypeptide. An exemplary polypeptide of this type can include, for example, a genetically modified version of an alcohol dehydrogenase such as, for example, a polypeptide encoded by a genetically modified adhE; or a genetically modified version of a phosphate acetyltransferase such as, for example, a polypeptide encoded by a genetically modified pta.

A decrease in catalytic activity can be quantitatively measured and described as a percentage of the catalytic activity of an appropriate wild type control. The catalytic activity exhibited by a genetically modified polypeptide can be, for example, no more than 95%, no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 3%, no more than 2%, no more than 1% of the activity, or 0% of the activity of a suitable wild type control.

Alternatively, a decrease in catalytic activity can be expressed as an appropriate change in a catalytic constant. For example, a decrease in catalytic activity may be expressed as at a decrease in k_{cat} such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the k_{cat} value of the enzymatic conversion.

A decrease in catalytic activity also may be expressed in terms of an increase in Kₘ such as, for example, an increase in Kₘ of at least two-fold, at least three-fold, at least four-fold, at least five-fold, at least six-fold, at least seven-fold, at least an eight-fold, at least nine-fold, at least 10-fold, at least 15-fold, at least 20-fold, at least 25-fold, at least 30-fold, at least 35-fold, at least 40-fold, at least 45-fold, at least 50-fold, at least 75-fold, at least 100-fold, at least 150-fold, at least 200-fold, at least 230-fold, at least 250-fold, at least 300-fold, at least 350-fold, or at least 400-fold.

An increase in catalytic activity can be quantitatively measured and described as a percentage of the catalytic activity of an appropriate wild type control. The catalytic activity exhibited by a genetically modified polypeptide can be, for example, at least 110%, at least 125%, at least 150%, at least 175%, at least 200% (two-fold), at least 250%, at least 300% (three-fold), at least 400% (four-fold), at least 500% (five-fold), at least 600% (six-fold), at least 700% (seven-fold), at least 800% (eight-fold), at least 900% (nine-fold), at least 1000% (10-fold), at least 2000% (20-fold), at least 3000% (30-fold), at least 4000% (40-fold), at least 5000% (50-fold), at least 6000% (60-fold), at least 7000% (70-fold), at least 8000% (80-fold), at least 9000% (90-fold), at least 10,000% (100-fold), or at least 100,000% (1000-fold) of the activity of an appropriate wild type control.

Alternatively, an increase in catalytic activity may be expressed as at an increase in k_{cat} such as, for example, at least a two-fold increase, at least a three-fold increase, at least a four-fold increase, at least a five-fold increase, at least a six-fold increase, at least a seven-fold increase, at least an eight-fold increase, at least a nine-fold increase, at least a 10-fold increase, at least a 15-fold increase, or at least a 20-fold increase in the k_{cat} value of the enzymatic conversion.

An increase in catalytic activity also may be expressed in terms of a decrease in Kₘ such as, for example, at least a two-fold decrease, at least a three-fold decrease, at least a four-fold decrease, at least a five-fold decrease, at least a six-fold decrease, at least a seven-fold decrease, at least an eight-fold decrease, at least a nine-fold decrease, at least a 10-fold decrease, at least a 15-fold decrease, or at least a 20-fold decrease in the Kₘ value of the enzymatic conversion.

In another aspect, the invention provides a method that includes introducing into a host cell a heterologous polynucleotide encoding a polypeptide that catalyzes conversion of isobutyraldehyde to isobutyrate operably linked to a promoter so that the modified host cell catalyzes conversion of isobutyraldehyde to isobutyrate. In various embodiments, the method includes further introducing into the cell one or more heterologous polynucleotides that encode genetic modifications and/or polypeptides described above. Such heterologous polynucleotides can include, for example, a genetic modification that decreases biosynthetic competition for isobutyraldehyde and thereby promotes accumulation of isobutyraldehyde for subsequent conversion to isobutyrate. Such heterologous polynucleotides also may encode a polypeptide that catalyzes a step in the conversion of a carbon source substrate to isobutyraldehyde.

In another aspect, the invention provides a method that includes incubating a recombinant cell as described herein in medium that comprises a carbon source under conditions effective for the recombinant cell to produce isobutyrate. Referring to FIG. 1(b), in some embodiments, the recombinant cell can convert glucose to isobutyrate by converting glucose to pyruvate, then converting pyruvate to isobutyraldenyde by, for example, the biosynthetic pathway illustrated in FIG. 1(b), then converting the isobutyraldehyde to isobutyrate through the activity of the heterologously-encoded polypeptide. In other embodiments, however, the isobutyraldehyde need not necessarily result from the metabolism of any particular feedstock through any particular biosynthetic pathway. For example, isobutyraldehyde may be provided directly to the recombinant cell in the culture medium. In other examples, the culture medium can include one or more intermediates of the biosynthetic pathway shown in FIG. 1(b) or any other biosynthetic pathway that produces isobutyraldehyde or feeds into the biosynthetic pathway shown in FIG. 1(b) to produce isobutyraldehyde. Thus, in various embodiments, the carbon source can include one or more of: glucose, Compound 6 of FIG. 1(b), Compound 7 of FIG. 1(b), Compound 8 of FIG. 1(b), Compound 9 of FIG. 1(b), and Compound 10 of FIG. 1(b).

Because isobutyrate is a commodity chemical, isobutyrate synthesis can be extended to the synthesis of other compounds. For example, isobutyrate may be a starting material for the synthesis of 3-hydroxy butyrate (Formula 5 of Reaction Scheme I, below) which can be dehydrated to produce methacrylic acid. Reaction Scheme I illustrates the synthesis of 3-hydroxy butyrate. Reaction Scheme I consists of four steps downstream of isobutyrate synthesis. Candidate genes coding enzymes for catalyzing each of these steps have been identified. Isobutyrate may be converted to isobutyryl-CoA (Formula 2 of Reaction Scheme I) by the enzyme butyryl-CoA:acetoacetate CoA-transferase (I, Reaction Scheme I) from, for example, Clostridium SB4 or Fusobacterium nucleatum. Isobutyryl-CoA may then be dehydrogenated to methylacrylyl-CoA (Formula 3 of Reaction Scheme I) by the enzyme 2-methylacyl-CoA dehydrogenase (II, Reaction Scheme I)—e.g., acdH from Streptomyces avermitilis or Acadsb from Rattus norvegicus. Methylacrylyl-CoA may then be hydrated to 3-hydroxy isobutyryl-CoA (Formula 4, Reaction Scheme I) by enoyl-CoA hydratase (III, Reaction Scheme I)—e.g., ECHS1 from Bos taurus or ech from Pseudomonas fluorescens. 3-hydroxy isobutyryl-CoA is converted to 3-hydroxy butyrate by 3-hydroxyisobutyryl-CoA hydrolase (IV, Reaction Scheme I)—e.g., Hibch from Rattus norvegicus.

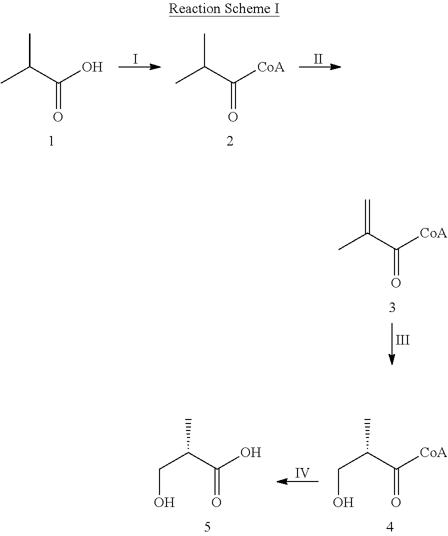

### Exemplary Enzymes Involved in Reaction Scheme I:

I: Enzyme: butyryl-CoA:acetoacetate CoA-transferase
Species: Clostridium SB4
Species: Fusobacterium nucleatum (Entrez Gene IDs 993155, 991616, or 992527, 992528)

II: Enzyme: 2-methylacyl-CoA dehydrogenase
Gene: acdH Accession Number: G-9098 (MetaCyc) Species: Streptomyces avermitilis
Gene: Acadsb Accession Number: G-9097 (MetaCyc) Species: Rattus norvegicus

III: Enzyme: short chain enoyl-CoA hydratase
Gene: ECHS1 Accession Number: G-9101 (MetaCyc) Species: Bos taurus

Enzyme: enoyl-CoA hydratase
Gene: ech Accession Number: G-9099 (MetaCyc) Species: Pseudomonas fluorescens

IV: Enzyme: 3-hydroxyisobutyryl-CoA hydrolase
Gene: Hibch Accession Number: G-9102 (MetaCyc) Species: Rattus norvegicus

The biosynthesis of other compounds from isobutyrate may be accomplished by co-culturing a recombinant cell described herein or a genetically modified cell described herein with a microbe that (a) can use isobutyrate as a sole carbon source and (b) possesses the metabolic ability to prodice the desired product, whether naturally or through genetic manipulation.

For example, engineered E. coli can be employed as the isobutyrate source during fermentation. To synthesize, for example, (S)-3-hydroxyisobutyrate, the engineered E. coli may be co-cultured with a strain of Pseudomonas putida (ATCC 21244) that can produce the S isomer of 3-hydroxyacid from isobutyrate with 48% yield. To synthesize the R isomer, one can co-culture the engineered E. coli with a yeast strain Candida rugosa (ATCC 10571). This species can produce 150 g/L (R)-3-hydroxyisobutyrate with a molar conversion yield of 81.8% from isobutyrate.

Alternatively, the biosynthesis of other compounds from isobutyrate may be accomplished by further modifying a recombinant cell described herein or a genetically modified cell described herein (collectively, a “biocatalyst”) by introducing isobutyrate-assimilation capability into the microbe so that a single biocatalyst is needed for biotransformation. For example, isobutyrate may be converted into isobutyryl-CoA by acyl-CoA synthetase (Acs). Isobutyryl-CoA may then be turned into methylacrylyl-CoA by acyl-CoA dehydrogenase (AcdH). Hydration of methylacrylyl-CoA by enoyl-CoA hydratase (Ech) can generate 3-hydroxy-isobutyryl-CoA, which may be hydrolyzed into 3-hydroxyisobutyrate by 3-hydroxyisobutyryl-CoA hydrolase (Hibch). 3-hydroxyisobutyrate may be oxidized to methylmalonate-semialdehyde by 3-hydroxyisobutyrate dehydrogenase (MmsB). Finally, the aldehyde may be converted to propanoyl-CoA by methylmalonate-semialdehyde dehydrogenase (MmsA). Propanoyl-CoA can enter central metabolism for biosynthesis to support growth. Acs, AcdH, Hibch, MmsB, and MmsA have been cloned from various organisms into E. coli and have demonstrated suitable expression levels and enzymatic activities in the new host. One can clone genes encoding such proteins and organize them into synthetic operons for optimal expression.

In contrast, Ech has not been cloned into and expressed in E. coli. No study has been performed to identify and characterize this catabolic enzyme in bacteria. From KEGG pathway database, for Pseudomonas putida KT2440, 10 enzymes (PP_{—}1412, PP_{—}1845, PP_{—}2136, PP_{—}2217, PP_{—}3283, PP_{—}3284, PP_{—}3358, PP_{—}3491, PP_{—}3726, PP_{—}3732 and PP_{—}4030) have been annotated to be Ech candidates. One can clone these proteins individually into the E. coli strain harboring other pathway enzymes and test the growth of transformants in medium with isobutyrate as the carbon source. This growth-based selection strategy can also be used to evolve any enzyme in the pathway if improved enzymatic activities in E. coli are desired. One can clone Acs, AcdH, Ech, and Hibch into the isobutyrate-producing E. coli strain. The resultant novel E. coli strain will be able to biosynthesize 3-hydroxybutyrate from glucose.

For any method disclosed herein that includes discrete steps, the steps may be conducted in any feasible order. And, as appropriate, any combination of two or more steps may be conducted simultaneously.

The present invention is illustrated by the following example. It is to be understood that the particular examples, materials, amounts, and procedures are to be interpreted broadly in accordance with the scope and spirit of the invention as set forth herein.

### EXAMPLES

### Example 1

### 1. Vector Construction

All cloning procedures were carried out in the E. coli strain XL10-gold (Stratagene, Agilent Technologies, Inc.; Santa Clara, Calif.). Primers (Table 2) were purchased from Eurofins MWG Operon. PCR reactions were performed with PHUSION High-Fidelity DNA polymerase (New England Biolabs, Inc.; Ipswich, Mass.) according to the manufacturer's instructions. The sequences of the plasmids produced from all cloning steps were verified using restriction mapping and DNA sequencing.

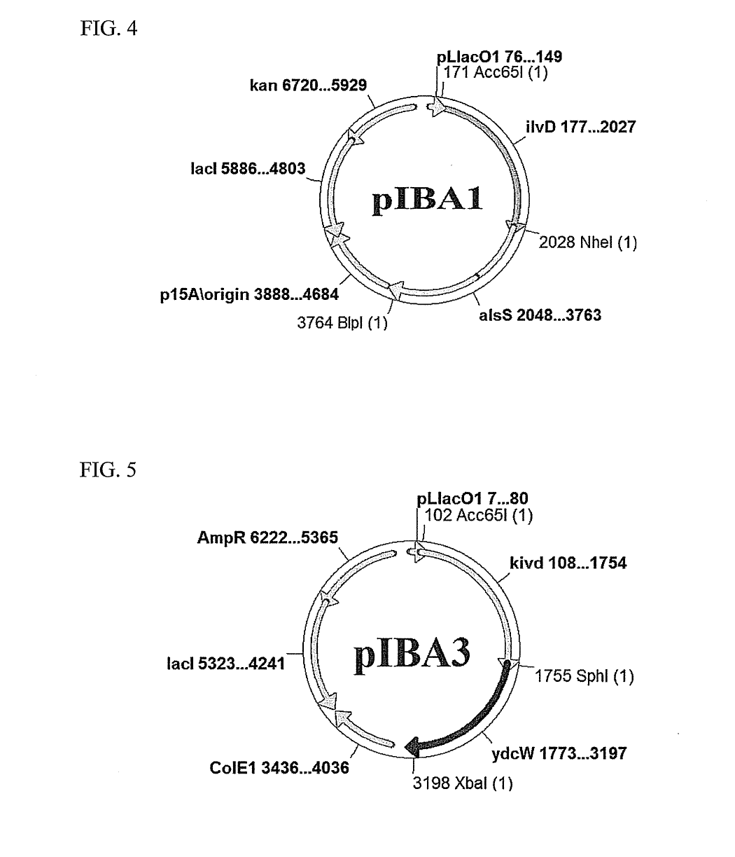

A gene fragment encoding lac repressor Lad was inserted respectively into the SacI site of plasmid pZE12 and pZA22 (Lutz and Bujard, 1997 Nucleic Acids Res. 25:1203-1210) to yield plasmid pZElac and pZAlac. E. coli genomic DNA was amplified with primers ilvd_accfwd and ilvd_nherev. The obtained ilvD gene fragment was digested with Acc65I and NheI. The Bacillus acetolactate synthase gene alsS was amplified from the genomic DNA of Bacillus subtilis with primers als_accremov/als_accremov_rev (to remove Acc65I site in alsS), as well as the flanking oligos als_nhefwd and als blprev using overlap PCR. The alsS PCR product was then digested with NheI and BlpI. Purified ilvD and alsS gene fragments were then ligated into pZAlac to create plasmid pIBA1 (see FIG. 4 for plasmid map).

The 2-ketoacid decarboxylase gene kivd was amplified from the genomic DNA of Lactococcus lactis (ATCC) using the primers kivd_accfwd and kivd_xbarev. The PCR product was digested with Acc65I and XbaI, and ligated into pZElac to yield plasmids pIBA2. Kivd was also amplified with kivd_accfwd and kivd_sphrev, and the PCR product was digested with Acc65I and SphI. YdcW was amplified from the E. coli genomic DNA with primers ydcw_sphfwd and ydcw_xbarev, which was then digested with SphI and XbaI. Purified kivd and ydcW gene fragments were then ligated into pZElac to create plasmid pIBA3 (see FIG. 5 for plasmid map). AldB was amplified from the E. coli genomic DNA with primers aldB_sphfwd and aldB_xbarev, digested with SphI and XbaI, and then inserted into pIBA3 to yield plasmid pIBA4. AldH was amplified from the E. coli genomic DNA with primers aldH_sphfwd and aldH_sphremov (to remove SphI site in aldH). And the PCR product was amplified again with primers aldH_sphfwd and aldB_xbarev, digested with SphI and XbaI, and then inserted into pIBA3 to yield plasmid pIBA5. Similarly, gabD was amplified with primers gabD_sphfwd and gabD_xbarev, and padA was amplified with primers padA_sphfwd and padA_xbarev from the E. coli genomic DNA. They were cloned into pIBA3 to yield plasmid pIBA6 and pIBA7. While kdh_{ba} was amplified from Burkholderia ambifaria (ATCC BAA-244) with primers kdh_{ba}_{_{—}}sphfwd and kdh_{ba}_{_{—}}xbarev, digested and ligated into into pIBA3 to yield plasmid pIBA8. And kdhₚₚ was amplified from Psuedomonas putida KT2440 (ATCC 47054D-5) with primers kdhₚₚ_{_{—}}sphfwd and kdhₚₚ_{_{—}}xbarev, digested and ligated into into pIBA3 to yield plasmid pIBA9.

PadA gene fragment was amplified using primers padA_bamfwd and padA_bamrev. After digestion with BamHI, the gene fragments were inserted into expression plasmid pQE9 (Qiagen, Inc. Valencia, Calif.) to yield pIBA10.

TABLE 2
Oligonucleotides for cloning.

| | | SEQ |
|---|---|---|
| Name | Sequence | ID NO: |
| als_nhefwd | ATGATCGCTAGCAGGAGAAATTAACTATGTTGACAAAAGCAACAAAAGAACA | 107 |
| als_blprev | GACTATGCTCAGCTTAGAGAGCTTTCGTTTTCATGAGTTC | 108 |
| als_accremov | AACATATCAAGCTGCCGGCACCCTTTCTAGAGATTTAGAGGA | 109 |
| als_accremov_rev | TCCTCTAAATCTCTAGAAAGGGTGCCGGCAGCTTGATATGTT | 110 |
| ilvd_accfwd | ATGATCGGTACCATGCCTAAGTACCGTTCCGCCA | 111 |
| ilvd_nherev | ATGATCGCTAGCTTAACCCCCCAGTTTCGATTTATCG | 112 |
| kivd_accfwd | GACTATGGTACCATGTATACAGTAGGAGATTACCTATTAG | 113 |
| kivd_sphrev | GACTATGCATGCTTATGATTTATTTTGTTCAGCAAATAG | 114 |
| kivd_xbarev | GACTATTCTAGATTATGATTTATTTTGTTCAGCAAATAG | 115 |
| ydcw_sphfwd | GACTATGCATGCAGGAGATATACCATGCAACATAAGTTACTGATTAACGGAG | 116 |
| ydcw_xbarev | GAC TATTCTAGATTAATGTTTAACCATGACGTGGCGGACG | 117 |
| aldB_sphfwd | GACTATGCATGCAGGAGATATACCATGACCAATAATCCCCCTTCAGCA | 118 |
| aldB_xbarev | GACTATTCTAGATTAGAACAGCCCCAACGGTTTATCCGA | 119 |
| aldH_sphfwd | GACTATGCATGCAGGAGATATACCATGAATTTTCATCATCTGGCTTACTGGCA | 120 |
| aldH_sphremov | GGCTTATCCAGATGGTTTTCAGTTCAGTGAATTTTTCAAGGGCGTGCAGGGATTTGTCGC | 121 |
| aldH_xbarev | GACTATTCTAGATTAGGCCTCCAGGCTTATCCAGATGGTTTTCAGTTCAG | 122 |
| gabD_sphfwd | GACTATGCATGCAGGAGATATACCATGAAACTTAACGACAGTAACTTATTCC | 123 |
| gabD_xbarev | GACTATTCTAGATTAAAGACCGATGCACATATATTTGA | 124 |
| padA_sphfwd | CTAGTAGCATGCAAGGAGATATACCATGACAGAGCCGCATGTAGCAGT | 125 |
| padA_xbarev | GACTATTCTAGATTAATACCGTACACACACCGACTTAGTT | 126 |
| kdh_{ba}_{_{—}}sphfwd | GACTATGCATGCAGGAGATATACCATGGCTAACGTGACTTATACGGATACG | 127 |
| kdh_{ba}_{_{—}}xbarev | GACTATTCTAGATTAGACCGCCATCACCGTCACC | 128 |
| kdhₚₚ_{_{—}}sphfwd | GACTATGCATGCAGGAGATATACCATGCCCCTCACAGGCAACCTG | 129 |
| kdhₚₚ_{_{—}}xbarev | GACTATTCTAGATTAGTCTTCCCGTTTACCATCAAGCA | 130 |
| padA_bamfwd | GACTATGGATCCATGACAGAGCCGCATGTAGCAGT | 131 |
| padA_bamrev | GACTATGGATCCTTAATACCGTACACACACCGACTTAGTT | 132 |

### 2. Fermentation Procedure and Product Analysis

### Host Strain

A wild type E. coli K-12 strain BW25113 (rrnB_{T14} ΔlacZ_{WJ16} hsdR514 ΔaraBAD_{AH33 ΔrhaBAD}_{LD78}) was transformed with pIBA1, and any plasmid from pIBA2 to pIBA9 for isobutyrate production.

The yqhD gene deletion strain was from the Keio collection (Baba et al., 2006 Mol. Syst. Biol. 2:10.1038). It was transformed with plasmid pCP20 to remove the kanamycin resistance marker. This ΔyqhD strain was transformed with pIBA1 and pIBA7 for isobutyrate production.

### Fermentation Process

Overnight cultures incubated in LB medium were diluted 25-fold into 5 mL M9 medium supplemented with 0.5% yeast extract and 4% glucose in 125-ml conical flasks. Antibiotics were added appropriately (ampicillin 100 mg/L and kanamycin 25 mg/L). 0.1 mM isopropyl-b-D-thiogalactoside (IPTG) was added to induce protein expression. The culture medium was buffered by addition of 0.5 g CaCO₃. Cultures were placed in a 30° C. shaker (250 rpm) and incubated for 48 hours.

Fermentation products were quantified by HPLC analysis with refractive index detection using an Agilent 1100 Capillary HPLC.

### 3. Enzymatic Assay

### Protein Overexpresion and Purification

pIBA10 was transformed into BL-21 E. coli host harboring the pREP4 plasmid (Qiagen; Valencia, Calif.). An overnight pre-culture was diluted 100-fold in 300 mL 2×YT rich medium containing 50 mg/L ampicillin, 25 mg/L kanamycin and 0.1 mM IPTG. Expression of recombinant protein was performed at 30° C. overnight.

The cell pellet was sonicated in 30 mL lysis buffer (250 mM NaCl, 2 mM DTT, 5 mM imidazole and 50 mM Tris pH 8.0). The cellular debris was removed by centrifugation at 15,000 RPM for 20 minutes. The supernatant was passed through a Ni-NTA column. Then the column was washed with 10 mL wash buffer (250 mM NaCl, 20 mM imidazole and 50 mM Tris pH 8.0) four times. Finally, the target protein was eluted with 10 mL elution buffer (250 mM NaCl, 250 mM imidazole and 50 mM Tris pH 8.0). The eluate was buffer-exchanged and concentrated into storage buffer (100 μM tris buffer, pH 8.0, and 20% glycerol) using AMICON ULTRA centrifugal filter (Millipore Corp.; Billerica, Mass.). Protein concentration was determined by measuring the UV absorbance at 280 nm (extinction coefficient, 75070 cm⁻¹ M⁻¹). The concentrated protein solution was aliquoted (100 μl) into PCR tubes and flash frozen at −80° C. for long term storage.

### Measurement of PadA Activity

Substrate isobutyraldehyde was purchased from Fisher Scientific International, Inc. (Hampton, N.H.), and NAD⁺ was from New England Biolabs, Inc., (Ipswich, Mass.). The reaction mixture contained 0.5 mM NAD⁺ and 0.2-4 mM isobutyraldehyde in assay buffer (50 mM NaH₂PO4, pH 8.0, 1 mM DTT) with a total volume of 80 μl. The reactions were started by adding 2 μl KIVD (final enzyme concentration 25 nM), and the generation of NADH was monitored at 340 nm (extinction coefficient, 6.22 mM⁻¹ cm⁻¹). Kinetic parameters (kₐₜ and Kₘ) were determined by fitting initial velocity data to the Michaelis-Menten equation using Origin software.

### Example 2

### Bacterial Strains and Plasmids

All the primers were from Eurofins MWG Operon (Huntsville, Ala.) and listed in Table 3. The E. coli strains used in this study were listed in Table 3, which were all derived from the wild type E. coli K-12 strain BW25113 with yqhD deletion. All cloning procedures were carried out in the E. coli strain XL10-gold (Stratagene; Santa Clara, Calif.). Plasmids pIBA1 and pIBA7 used to produce isobutyrate were from previous work (Zhang et al., 2011 ChemSusChem 4:1068-1070). To build the pIBA1 plasmid 1 carrying two copies of padA, the padA gene was amplified by PCR with oligos padA_Saclfwd and padA_SacIrev, digested with SacI and then ligated into pIBA7 to create pIBA11. In pIBA11, the additional copy of padA is in the same operon with ampicillin resistance gene bla, under the regulation of a constitutive promoter. P1 phages of adhE, adhP, eutG, yiaY and yjgB were obtained from the Keio collection (Baba et al., 2006 Mol. Syst. Biol. 2:10.1038). The phages were used to transfect the IBA1 strain to construct double knockout strains. All the knockout strains were then transformed with pCP20 plasmid to remove the kanamycin marker. The correct knockouts were verified by PCR. To produce isobutyrate, each strain was transformed with plasmids pIBA1 plus pIBA7, or pIBA1 plus pIBA11.

TABLE 3
Strains, plasmids and primers used in this study

| Name | Relevant genotype | Reference |
|---|---|---|
| Strains | | |
| BW25113 | rrnB_{T14} ΔlacZ_{WJ16} hsdR514 ΔaraBAD_{AH33} ΔrhaBAD_{LD78} | Datsenko and Wanner, |
| | | 2000 Proc. Natl. Acad. |
| | | Sci. U.S.A. 97: 6640-6645 |
| IBA1 | BW25113 ΔyqhD | Zhang et al., 2011 |
| | | ChemSusChem 4: 1068-1070 |
| IBA11 | BW25113 ΔyqhD ΔadhE | This work |
| IBA12 | BW25113 ΔyqhD ΔadhP | This work |
| IBA13 | BW25113 ΔyqhD ΔeutG | This work |
| IBA14 | BW25113 ΔyqhD ΔyiaY | This work |
| IBA15 | BW25113 ΔyqhD ΔygjB | This work |
| IBA1-1C | BW25113 ΔyqhD + pIBA1 and pIBA7 | This work |
| IBA11-1C | BW25113 ΔyqhD ΔadhE + pIBA1 and pIBA7 | This work |
| IBA12-1C | BW25113 ΔyqhD ΔadhP + pEBA1 and pIBA7 | This work |
| IBA13-1C | BW25113 ΔyqhD ΔeutG + pIBA1 and pIBA7 | This work |
| IBA14-1C | BW25113 ΔyqhD ΔyiaY + pIBA1 and pIBA7 | This work |
| IBA15-1C | BW25113 ΔyqhD ΔygiB + pIBA1 and pIBA7 | This work |
| IBA1-2C | BW25113 ΔyqhD + pIBA1 and pIBA11 | This work |
| IBA15-2C | BW25113 ΔyqhD ΔygjB + pIBA1 and pIBA11 | This work |
| plasmids | | |
| pIBA1 | p15A ori, Kan^{R}, P_{L}lacO₁::alsS ilvD | Zhang et al., 2011 |
| | | ChemSusChem 4: 1068-1070 |
| pIBA7 | ColE1 ori, Amp^{R}, P_{L}lacO₁::kivD padA | Zhang et al., 2011 |
| | | ChemSusChem 4: 1068-1070 |
| pIBA11 | ColE1 ori, Amp^{R}, P_{L}lacO₁::kivD padA padA | This work |
| Primers | | SEQ ID NO: |
| adhEKOC-F | TTGCTTACGCCACCTGGAAGT | 133 |
| adhEKOC-F | GAACGGTCGCATGAGCAGAAAG | 134 |
| adhPKOC-F | TGACGATAATTTCTGGCAAGC | 135 |
| adhPKOC-R | GCAGGCTGACATTAAGTTCGT | 136 |
| eutGKOC-F | AGATTTGGCCTGCGGTGAAA | 137 |
| eutGKOC-R | CTGTTAGTTGTTATTTATTGGCGG | 138 |
| yiaYKOC-F | CATTTATTGCGCGACGCATTAT | 139 |
| yiaYKOC-R | ATAGCGGGCTTTTAACTTGAGG | 140 |
| yjgBKOC-F | CACTGAAGAGGTATGCGGAAAA | 141 |
| yjgBKOC-R | CTGGGCATTTTATGCCGGTAG | 142 |
| padA_SacIfwd | ctagtagagctcaAGGAGATATACCatgacagagccgcatgtagcagt | 143 |
| padA_SacIrev | GACTATGAGCTCTTAATACCGTACACACACCGACTTAGTT | 144 |

### Cell Cultivation and Shake Flask Fermentation.

Unless otherwise stated, cells were grown in test tubes at 37° C. in 2XYT rich medium (16 g/L Bacto-tryptone, 10 g/L yeast extract and 5 g/L NaCl) supplemented with 100 mg/L ampicillin and 50 mg/L kanamycin. 200 μl of overnight cultures incubated in 2XYT medium were transferred into 5 ml M9 minimal medium supplemented with 5 g/L yeast extract, 40 g/L glucose, 100 mg/L ampicillin and 50 mg/L kanamycin in 125 ml conical flasks. Isopropyl-β-D-thiogalactoside (IPTG) was added at a concentration of 0.1 mM to induce protein expression. The fermentation broth was buffered by the presence of 0.5 g CaCO₃. Fermentation cultures were placed at 30° C. in a shaker with a speed of 250 rpm.

### Culture Media for Fermentor.

The following composition is the seeding medium for E. coli culture, in grams per liter: glucose, 10; (NH₄)₂SO₄, 1.8; K₂HPO₄, 8.76; KH₂PO₄, 2.4; sodium citrate, 1.32; yeast extract, 15; ampicillin, 0.1; kanamycin, 0.05. Fermentation media for bioreactor cultures contained the following composition, in grams per liter: glucose, 30; (NH₄)₂SO₄, 3; K₂HPO₄, 14.6; KH₂PO₄, 4; sodium citrate, 2.2; yeast extract, 25; MgSO₄.7H₂O, 1.25; CaCl₂.2H₂O, 0.015, calcium pantothenate, 0.001; Thiamine, 0.01; ampicillin, 0.1; kanamycin, 0.05; and 1 mL/L of trace metal solution. Trace metal solution contained, in grams per liter: NaCl, 5; ZnSO₄.7H₂O, 1; MnCl₂.4H₂O, 4; CuSO₄.5H₂O, 0.4; H₃BO₃, 0.575; Na2MoO4.2H₂O, 0.5; FeCl₃.6H₂O, 4.75; 6NH₂SO₄, 12.5 mL. The feeding solution contained, in grams per liter: glucose, 600; (NH₄)₂SO₄, 5; MgSO₄.7H₂O, 1.25; yeast extract, 5; CaCl₂.2H₂O, 0.015; calcium pantothenate, 0.001; Thiamine, 0.01; ampicillin, 0.1; kanamycin, 0.05, 0.2 mM of IPTG; and 1 mL/L of trace elements.

### Fermentor Culture Conditions.

Cultures of E. coli were performed in a 1.3 L Bioflo 115 fermentor (NBS; Edison, N.J.) using a working volume of 0.6 L. The fermentor was inoculated with 10% of overnight pre-culture with seeding medium and then the cells were grown at 37° C., 30% dissolved oxygen (DO) level, and pH 7.0. After OD₆₀₀ reached 8.0, 0.2 mM IPTG was added and the temperature was shifted to 30° C. to start isobutyrate production. The pH was controlled at 7.0 by automatic addition of 10 M sodium hydroxide solution, 50% ammonia hydroxide, or 200 g/L calcium hydroxide suspension, respectively. Air flow rate was maintained at 1 vvm in the whole process. DO was maintained at about 10% with respect to air saturation by adjusting stirring speed (from 300 to 800 rpm). The glucose level in the fermentor was kept around 10 g/L by adding feeding medium automatically. When DO went over 40% and isobutyrate level did not increase, the fermentation process was stopped. Fermentation samples at different time points were collected to determinate optical density and metabolite concentration.

### Metabolite Analysis and Dry Cell Weight Determination.

Fermentation products were analyzed using an Agilent 1260 Infinity HPLC equipped with an Aminex HPX 87H column (Bio-Rad; Hercules, Calif.) and a refractive-index detector. The mobile phase was 5 mM H₂SO₄ with a flow rate 0.6 mL/min. The column temperature and detection temperature were 35° C. and 50° C., respectively. Cell dry weight was determined by filtering 5 mL culture through a 0.45 μm glass fiber filter (Michigan Fiberglass Sales; St. Clair Shores, Mich.). After removal of medium, the filter was washed with 15 mL of MilliQ water, dried in an oven and then weighed. Cell dry weight was determined in triplicate.

### Example 3

### Cloning Procedure

BKDH enzyme complex genes were amplified from Pseudomonas Putida KT2440 genomic DNA with primers bkdh_ecofwd (TGCATCGAATTCAGGAGAAATTAACTAT GAACGAGTACGCCCCCCTGCGTTTGC (SEQ ID N0:145)) and bkdh_hindrev (TGCATCAAGCTTTCAGATATGCAAGGCGTGGCCCAG (SEQ ID NO:146)). The PCR product was then digestion with EcoRI and HindIII, and inserted into pZE12 to make pIBA16.

The tesA gene was amplified from E. coli strain K12 genomic DNA using the primer pair TesA_HindIII_F (GGGCCCAAGCTTAGGAGAAATTAACTATGATGAACTTCAAC AATGTTTTCCG (SEQ ID NO:147)) and TesA_Xba1_R (GGGCCCTCTAGATTATGAGT CATGATTTACTAAAGGCT (SEQ ID NO:148)); tesB was amplified with primer pair TesB_HindIII_F (GGGCCCAAGCTTAGGAGAAATTAACTATGATGAGTCAGGCGCT AAAAAATTTACT (SEQ ID NO:149)) and TesB_Xba1_R (GGGCCCTCTAGATTAATT GTGATTACGCATCACCCCTT (SEQ ID NO:150)). After PCR, the DNA fragments were purified and digested using the restriction enzymes HindIII and Xba1. The digested fragments containing tesA were inserted into pIBA16 to make pIBA17; the digected fragments containing tesB were inserted into pIBA16 to make pIBA18.

### Fermentation Process

Overnight cultures incubated in LB medium were diluted 25-fold into 5 mL M9 medium supplemented with 0.5% yeast extract and 4% glucose in 125-mL conical flasks. Antibiotics were added appropriately (ampicillin 100 mg/L and kanamycin 25 mg/L). 0.1 mM isopropyl-b-D-thiogalactoside (IPTG) was added to induce protein expression. The culture medium was buffered by addition of 0.5 g CaCO₃. Cultures were placed in a 30° C. shaker (250 rpm) and incubated for 48 hours.

Fermentation products were quantified by HPLC analysis with refractive index detection using an Agilent 1100 Capillary HPLC. Results are shown in Table 4.

TABLE 4
Production of isobutyrate with the new pathway.

| Strain | Modification | Isobutyrate (g/L) |
|---|---|---|
| pIBA1 + pIBA16 | BKDH | 5.61 ± 0.67 |
| pIBA1 + pIBA17 | BKDH + TesA | 6.47 ± 0.22 |
| pIBA1 + pIBA18 | BKDH + TesB | 8.62 ± 0.10 |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1
SeqID: AP_002012.1 GI: 89108232
Protein name: phenylacetaldehyde dehydrogenase PadA
[Escherichia coli str. K-12 substr. W3110].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadhltrvt lelggknpai vlkdadpqwv ieglmtgsfl |
| 301 | nqgqvcaass riyieaplfd tivsgfeqav kslqvgpgms pvaqinplvs rahcdkvcsf |
| 361 | lddaqaqqae lirgsngpag egyyvaptlv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldgwc etksvcvry |

SEQ ID NO: 2
SeqID: AP_004204.1 GI: 89110424
Protein name: acetaldehyde dehydrogenase AldB
[Escherichia coli str. K-12 substr. W3110].

| | |
|---|---|
| 1 | mtnnppsaqi kpgeygfplk lkarydnfig gewvapadge yyqnltpvtg qllcevassg |
| 61 | krdidlalda ahkvkdkwah tsvqdraail fkiadrmeqn lellataetw dngkpirets |
| 121 | aadvplaidh fryfascira qeggisevds etvayhfhep lgvvgqiipw nfpllmaswk |
| 181 | mapalaagnc vvlkparltp lsvlllmeiv gdllppgvvn vvngaggvig eylatskria |
| 241 | kvaftgstev gqqimqyatq niipvtlelg gkspniffad vmdeedaffd kalegfalfa |
| 301 | fnqgevctcp sralvqesiy erfmerairr vesirsgnpl dsvtqmgaqv shgqletiln |
| 361 | yidigkkega dvltggrrkl legelkdgyy leptilfgqn nmrvfqeeif gpvlavttfk |
| 421 | tmeealelan dtqyglgagv wsrngnlayk mgrgiqagrv wtncyhaypa haafggykqs |
| 481 | gigrethkmm lehyqqtkcl lvsysdkplg lf |

SEQ ID NO: 3
SeqID: AP_001926.1 GI: 89108146
Protein name: gamma3-hydroxypropionaldehyde dehydrogenase AldH
[Escherichia coli str. K-12 substr.
W3110].

| | |
|---|---|
| 1 | mnfhhlaywq dkalslaien rlfingeyta aaenetfetv dpvtqaplak iargksvdid |
| 61 | ramsaargvf ergdwslssp akrkavinkl adlmeahaee lalletldtg kpirhslrdd |
| 121 | ipgaarairw yaeaidkvyg evattsshel amivrepvgv iaaivpwnfp llltcwklgp |
| 181 | alaagnsvil kpseksplsa irlaglakea glpdgvlnvv tgfgheagqa lsrhndidai |
| 241 | aftgstrtgk qllkdagdsn mkrvwleagg ksanivfadc pdlqqaasat aagifynqgq |
| 301 | vciagtrlll eesiadefla llkqqaqnwq pghpldpatt mgtlidcaha dsvhsfireg |
| 361 | eskgqllldg rnaglaaaig ptifvdvdpn aslsreeifg pvlvvtrfts eeqalqland |
| 421 | sqyglgaavw trdlsrahrm srrlkagsvf vnnyndgdmt vpfggykqsg ngrdkslhal |
| 481 | ekftelktiw islea |

SEQ ID NO: 4
SegID: NP_417147.1 GI: 16130575
Protein name: succinate semialdehyde dehydrogenase GabD
[Escherichia coli str. K-12 substr. MG1655].

| | |
|---|---|
| 1 | mklndsnlfr qqalingewl danngeaidv tnpangdklg svpkmgadet raaidaanra |
| 61 | lpawraltak eratilrnwf nlmmehqddl arlmtleqgk plaeakgeis yaasfiewfa |
| 121 | eegkriygdt ipghqadkrl ivikqpigvt aaitpwnfpa amitrkagpa laagctmvlk |
| 181 | pasqtpfsal alaelairag vpagvfnvvt gsagavgnel tsnplvrkls ftgsteigrq |
| 241 | lmeqcakdik kvslelggna pfivfddadl dkavegalas kfrnagqtcv canrlyvqdg |
| 301 | vydrfaeklq qavsklhigd gldngvtigp lidekavakv eehiadalek garvvcggka |
| 361 | herggnffqp tilvdvpana kvskeetfgp laplfrfkde adviaqandt efglaayfya |
| 421 | rdlsrvfrvg ealeygivgi ntgiisneva pfggikasgl gregskygie dyleikymci |
| 481 | gl |

SEQ ID NO: 5
SeqID: YP_775718.1 GI: 115358580
Protein name: α-ketoglutaric semialdehyde dehydrogenase
[Burkholderia ambifaria AMMD].

| | |
|---|---|
| 1 | manvtytdtq llidgewvda asgktidvvn patgkaigkv ahagiadldr alaaaqrgfe |
| 61 | awrkvpaner aatmrkaaal vreradaiaq lmtqeqgkpl tearvevlsa adiiewfade |
| 121 | grrvygrivp prnlgaqqmv vkepvgpvaa ftpwnfpvnq vvrklsaala tgcsflvkap |
| 181 | eetpaspaal lrafvdagvp agviglvyge paeissylia hpvirkvtft gstpvgkqla |
| 241 | alagqhmkra tmelgghapv ivaedadval avkaaggakf rnagqvcisp trflvhnsir |
| 301 | deftralvqh aeglkigngl eegttlgala nprrltamas vvenarkvga sietggerig |
| 361 | segnffaptv ianvpleadv fnnepfgpva airgfdkled aiaeanrlpf glagyaftrs |
| 421 | fanvhllsqr levgmlwinq patpwpempf ggvkdsgygs eggpealepy lvtksvtvma |
| 481 | v |

SEQ ID NO: 6
SegID: NP_743416.1 GI: 26987991
Protein name: ketoglutarate semialdehyde dehydrogenase
[Pseudomonas putida KT2440].

| | |
|---|---|
| 1 | mpltgnllig qrpvtgsrda iraidpttgq tlepaylggt gehvaqacal awaafdayre |
| 61 | tsleqraefl eaiatqieal gdalidravi etglpkariq gergrtctql rtfartvrag |
| 121 | ewldvridsa lperqplpra dlrqrqvalg pvavfgasnf plafsvaggd tasalaagcp |
| 181 | vvvkahsahp gtselvgqav aqavkqcglp egvfsllygs grevgialvs dprikavgft |
| 241 | gsrsggmalc qaagarpepi pvyaemssin pvflfdaalq araealaqgf vasltqgagq |
| 301 | fctnpglvia rqgpalqrfi taaagyvqqg aaqtmltpgi fsayqagiaa ladnphaqai |
| 361 | tsgqagqgpn qcqaqlfvtq aeafladpal qaevfgaasl vvactddeqv rqvaehlegq |
| 421 | ltatlqldea didsarallp tlerkagril vngwptgvev cdamvhggpf patsdartts |
| 481 | vgtaailrfl rpvcyqdvpd allpqalkhg nplqlrrlld gkred |

SEQ ID NO: 7
SegID: NP_415961.1 GI: 16129403
Protein name: γ-aminobutyraldehyde dehydrogenase YdcW
[Escherichia coli str. K-12 substr. MG1655].

| | |
|---|---|
| 1 | mqhkllinge lvsgegekqp vynpatgdvl leiaeasaeq vdaavraada afaewgqttp |
| 61 | kvraecllkl advieengqv faelesrncg kplhsafnde ipaivdvfrf fagaarclng |
| 121 | laageylegh tsmirrdplg vvasiapwny plmmaawkla palaagncvv lkpseitplt |
| 181 | alklaelakd ifpagvinil fgrgktvgdp ltghpkvrmv sltgsiatge hiishtassi |
| 241 | krthmelggk apvivfddad ieavvegvrt fgyynagqdc taacriyaqk giydtivekl |
| 301 | gaavatlksg apddestelg plsslahler vgkaveeaka tghikvitgg ekrkgngyyy |
| 361 | aptllagalq ddaivqkevf gpvvsvtpfd neeqvvnwan dsqyglassv wtkdvgrahr |
| 421 | vsarlqygct wvnthfmlvs emphggqkls gygkdmslyg ledytvvrhv mvkh |

SEQ ID NO: 8
SeqID: ZP_06662203.1 GI: 293433775
Protein name: phenylacetaldehyde dehydrogenase
[Escherichia coli B088].

| | |
|---|---|
| 1 | mmtephvavl sqvqqfldrq hglyidgrpg paqsekrlai fdpatgqeia stadaneadv |
| 61 | dnavmsawra fvsrrwagrl paererillr fadlveqhse elaqletleq gksiaisraf |
| 121 | evgctlnwmr ytaglttkia gktldlsipl pqgaryqawt rkepvgvvag ivpwnfplmi |
| 181 | gmwkvmpala agcsivikps ettpltmlry aelaseagip dgvfnvvtgs gavcgaalts |
| 241 | hphvakisft qstatgkgia rtaadhltrv tlelggknpa ivlkdadpqw vieglmtgsf |
| 301 | lnqgqvcaas sriyieaplf dtivsgfeqa vkslqvgpgm spvaqinplv srahcdkvcs |
| 361 | flddaqaqqa elirgsngpa gegyyvaptl vvnpdaklrl treevfgpvv nlvrvadgee |
| 421 | alqlandtey gltasvwtqn lsqaleysdr lqagtvwvns htlidanlpf ggmkqsgtgr |
| 481 | dfgpdwldgw cetksvcvry |

SEQ ID NO: 9
SeqID: AP_002012.1 GI: 89108232
Protein name: phenylacetaldehyde dehydrogenase
[Escherichia coli str. K-12 substr. W3110].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadhltrvt lelggknpai vlkdadpqwv ieglmtgsfl |
| 301 | nqgqvcaass riyieaplfd tivsgfeqav kslqvgpgms pvaqinplvs rahcdkvcsf |
| 361 | lddaqaqqae lirgsngpag egyyvaptiv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldgwc etksvcvry |

SEQ ID NO: 10
SeqID: ZP_07162545.1 GI: 300948446
Protein name: aldehyde dehydrogenase family protein
[Escherichia coli MS 116-1].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkivg ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadhltrvt lelggknpai vlkdadpqwv ieglmtgsfl |
| 301 | nqgqvcaass riyieaplfd tivsgfeqav kslqvgpgms pvaqinplvs rahcdkvcsf |
| 361 | lddaqaqqae lirgsngpag egyyvaptlv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldgwc etksvcvry |

SEQ ID NO: 11
SegID: YP_002386828.1 GI: 218553915
Protein name: phenylacetaldehyde dehydrogenase
[Escherichia coli IAI1].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadhltrvt lelggknpai vlkdadpqwv ieglmtgsfl |
| 301 | nqgqvcaass riyieaplfd tlvsgfeqav kslqvgpgms pvaqinpvvs rahcdkvcsf |
| 361 | lddaqaqqae lirgsngpag egyyvaptiv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldgwc etksvcvry |

SEQ ID NO: 12
SegID: ZP_07103419.1 GI: 300823287
Protein name: aldehyde dehydrogenase family protein
[Escherichia coli MS 119-7].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsipfp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadhltrvt lelggknpai vlkdadpqwv ieglmtgsfl |
| 301 | nqgqvcaass riyieaplfd tlvsgfeqav kslqvgpgms pvaqinplvs rahcdkvcsf |
| 361 | lddaqaqqae lirgsngpag egyyvaptlv vnpdaklrlt reevfgpvvn lvrvadgeea |
| 421 | lglandteyg ltasvwtqnl sgaleysdrl qagtvwvnsh tlidanlpfg gmkgsgtgrd |
| 481 | fgpdwldgwc etksvcvry |

SEQ ID NO: 13
SegID: ZP_07561137.1 GI: 307280084
Protein name: Phenylacetaldehyde dehydrogenase
[Escherichia coli KO11].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadrltrvt lelggknpai vlkdadpqwv ieglmtgsfl |
| 301 | nqgqvcaass riyieaplfd tivsgfeqav kslqvgpgms pvaqinplvs rahcdkvcsf |
| 361 | lddaqaqqae lirgsngpag egyyvaptlv vnpdaklrlt reevfgpvvn lvrvadgeea |
| 421 | lglandteyg ltasvwtqnl sgaleysdrl qagtvwvnsh tlidanlpfg gmkqsgtgrd |
| 481 | fgpdwldgwc etksvcvry |

SEQ ID NO: 14
SeqID: YP_003036452.1 GI: 253773621
Protein name: Phenylacetaldehyde dehydrogenase
[Escherichia coli BL21-Gold(DE3)pLysS AG].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqlepleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadrltrvt lelggknpai vlkdadpqwv ieglmtgsfl |
| 301 | nqgqvcaass riyieaplfd tlvsgfeqav kslqvgpgms pvaqinplvs rahcgkvcsf |
| 361 | lddaqaqqae lirgsngpag egyyvaptlv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldgwc etksvcvry |

SEQ ID NO: 15
SeqID: ZP_07182441.1 GI: 301017791
Protein name: aldehyde dehydrogenase family protein
[Escherichia coli MS 69-1].

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg svcgaaltsh |
| 241 | phvakisftg statgkgiar taadrltrvt lelggknpai vlkdadpqwv ieglmtgsfl |
| 301 | nqgqvcaass riyieaplfd tivsgfeqav kslqvgpgms pvaqinplvs rahcdkvrsf |
| 361 | lddaqaqqae lirgasgpag egyyvaptiv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldgwc etksvcvry |

SEQ ID NO: 16
SeqID: ZP_07099886.1 GI: 300819696
Protein name: aldehyde dehydrogenase family protein
[Escherichia coli MS 107-1]

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadrltrvt lelggknpai vlkdadpqwv ieglmtgsfl |
| 301 | nqgqvcaass riyieaplfd tivsgfeqav kslqvgpgms pvaqinplvs rahcdkvcsf |
| 361 | lddaqaqqae lirglngpag egyyvaptlv vnpdaklrlt reevfgpvvn lvrvadgeea |
| 421 | lqlandteyg ltasvwtqnl sqaleysdrl qagtvwvnsh tlidanlpfg gmkqsgtgrd |
| 481 | fgpdwldswr etksvcvry |

SEQ ID NO: 17
SeqID: ZP_07099886.1 GI: 300819696
Protein name: aldehyde dehydrogenase family protein
[Escherichia coli MS 107-1]

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phvakisftg statgkgiar taadrltrvt lelggknpai vlkdadpqwv ieglmtgsfl |
| 301 | nqgqvcaass riyieaplfd tivsgfeqav kslqvgpgms pvaqinplvs rahcdkvcsf |
| 361 | lddaqaqqae lirglngpag egyyvaptlv vnpdaklrlt reevfgpvvn lvrvadgeea |
| 421 | lqlandteyg ltasvwtqnl sqaleysdrl qagtvwvnsh tlidanlpfg gmkgsgtgrd |
| 481 | fgpdwldswr etksvcvry |

SEQ ID NO: 18
SeqID: YP_002382755.1 GI: 218548964
Protein name: phenylacetaldehyde dehydrogenase
[Escherichia fergusonii ATCC 35469]

| | |
|---|---|
| 1 | mtephvavls qvqqfldrqh glyidgrpgp aqsekrlaif dpatgqeias tadaneadvd |
| 61 | navmsawraf vsrrwagrlp aererillrf adlveqhsee laqletmeqg ksiaisrafe |
| 121 | vgctlnwmry taglttkiag ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phiakisftg statgkgiar taadrltrvt lelggknpai vlkdadpqwv ieglmtgsfl |
| 301 | nqgqvcaass riyieaplfd nlvsgfeqav kslqvgpgms pvaqinplvs rahcdkvrsf |
| 361 | lddaqaqkae lirgasgpag egyyvaptlv vnpdaklrlt reevfgpvvn lvrvadgeea |
| 421 | lqlandteyg ltasvwtqnl tqaleysdrl qagtvwvnsh tlidanlpfg gmkqsgtgrd |
| 481 | fgpdwldgwc etksvcvry |

SEQ ID NO: 19
SeqID: ZP_03064187.1 GI: 194431897
Protein name: phenylacetaldehyde dehydrogenase
[Escherichia fergusonii ATCC 35469]

| | |
|---|---|
| 1 | mtephvavls qvqqfldrqh glyidgrpgp aqsekrlpif npatgqeias tadaneadvd |
| 61 | navmsawraf vsrrwagrlp aererillrf adlveqhsee laqletleqg ksiaisrafe |
| 121 | vgctlnwmry taglttkiag ktldlsiplp qgaryqvwtr kepvgvvagi vpwnfplmig |
| 181 | mwkvmpalaa gcsivikpse ttpltmlrva elaseagipd gvfnvvtgag avcgaaltsh |
| 241 | phvakisftg statgkgiar taadrltrvt lelggknpai vlkdadpqwv ieglmtgsfl |
| 301 | nqgqvcaasl riyieaplfd tlvsgfeqav kslqvgpgms pvaqinplvs rahcdkvcsf |
| 361 | lddaqaqqae lirgsngpag egyyvaptlv vnpdaklrlt reevfgpvvn lvrvadgeea |
| 421 | lqlandteyd ltasvwtqnl sraleysdrl qagtvwvnsh tlidanlpfg gmkqsgtgrd |
| 481 | fgpdwldgwc etksvcvry |

SEQ ID NO: 20
SeqID: YP_003612657.1 GI: 296102511
Protein name: phenylacetaldehyde dehydrogenase
[Enterobacter cloacae subsp. cloacae ATCC 13047].

| | |
|---|---|
| 1 | msesqvaiqp gvqqfldrhh glwiegrqaa sesekrlniy npatgevias tadasvddvd |
| 61 | ravmsgwraf varnwagklp aererillrf adlveqhsee laqletleqg ksinisrlfe |
| 121 | vgctlnwmry taglttkitg ktldlsiplp qgaryqawtr kepvgvvagi vpwnfplmig |
| 181 | mwkvmpalaa gcsivikpse ttpltmlrva elaseagipd gvfnvvtgsg avcgaaltsh |
| 241 | phiakvsftg statgkqiar aaadtltgvt lelggknpai vlkdadpawv ieglmtgsfl |
| 301 | nqgqvcaass riyieaplfd tlvsgfeqav kslsvgpgms peafinplvs rahcdkvqtf |
| 361 | ldeaksrnae litgnrgpdg kgyyisptlv vnpdpglrlt reevfgpvvn lvrvadgeea |
| 421 | ltlandteyg ltasvwtqni skaleytdrl qagtvwvnsh tlidanlpfg gmkqsgtgrd |
| 481 | fgpdwldgwc etksvcvry |

SEQ ID NO: 21
SeqID: ZP_05967560.2 GI: 288549605
Protein name: phenylacetaldehyde dehydrogenase
[Enterobacter cancerogenus ATCC 35316]

| | |
|---|---|
| 1 | makplldcin ilrritkeyp msesqvavlp cvqqfldrhh glwiegreva sdgekrlnvy |
| 61 | npatgevias tadasvddvd ravmsgwraf vsrswagtlp aererillhf adlveqhgee |
| 121 | laqletleqg ksinisrafe vgctlnwmry taglttkiag ktldlsiplp qgaryqawtr |
| 181 | kepvgvvagi vpwnfplmig mwkvmpalaa gcsivikpse ttpltllrva elaseagipd |
| 241 | gvfnvitgsg atcgaaltsh priakvsftg statgkqiar taaetltgvt lelggknpai |
| 301 | vlkdadpawv ieglmtgsfl nqgqvcaass riyieaplfd tlvsgfeqav kslsvgpgms |
| 361 | pqafinplvs rahcdkvqtf ldeaasrkae lisgsrgpdg kgyyvsptlv vnpdaslrlt |
| 421 | reevfgpvvn lvrvadgeea lrlandteyg ltasvwtqni skaleytdrl qagtvwvnsh |
| 481 | tlidanlpfg gmkqsgtgrd fgpdwldgwc etksvcvry |

SEQ ID NO: 22
SeqID: YP_001335117.1 GI: 152970008
Protein name: phenylacetaldehyde dehydrogenase
[Klebsiella pneumoniae subsp. pneumoniae MGH 78578]

| | |
|---|---|
| 1 | mstsqialla svqqfldrqh glyidgapca aqsenrltvw dpatgqaiat tadaspadvd |
| 61 | ravmsawraf vdrrwagrtp adrerillrf adlveqhgee laqletleqg ksiaisrafe |
| 121 | vgctlnwmry taglttkisg rtldvsipfp qgaryqawtk kepvgvvagi vpwnfplmig |
| 181 | mwkvmpalaa gcsivikpse ttpltllrva elatqagipd gvfnvvtgsg agcgaaltah |
| 241 | pqvakvsftg statgkqiar vaadrltrvt lelggknpai vlkdadpqwv ieglmtgsfl |
| 301 | nqgqvcaass riyieaplfd tlvsgfeqav kslqvgpgmq etaqinpvvs rahcdkvaay |
| 361 | leearqqkae lisgsagpda ggyyipptlv vnpdaglrls reevfgpvvn lvrvadgeea |
| 421 | lrlandsdfg ltasvwtrdl tqalnytdrl qagtvwvnsh tlidanlpfg gmkqsgtgrd |
| 481 | fgpdwldgwc etksvcvry |

SEQ ID NO: 23
SeqID: YP_003941961.1 GI: 311279730
Protein name: Aldehyde Dehydrogenase
[Enterobacter cloacae SCF1]

| | |
|---|---|
| 1 | msdsqvaile rvqqflarqh glfidgaqqp srsdkrltvw npatgeaiat tadansadvd |
| 61 | savmsawraf vdrrwagrlp aererillrf tdlveqhsee laqletleqg ksinisrafe |
| 121 | vactlnwmry taglttkitg qtldvsiplp pgaryqawtr kepvgvvagi vpwnfplmig |
| 181 | mwkvmpalaa gcsivikpse ttpltllrva elaveagipe gvfnvvtgsg aecgaaltsh |
| 241 | phvakvsftg stqtgkqiar taadrltrvt lelggknpai vlkdadpqwv ieglmmgsfl |
| 301 | nqgqvcaass riyieaplfd tlvtgfeqav kslsvgpgms eaaqinplas rahcdkvaaf |
| 361 | lhdaqqhhae lihgnegpgg qgyyirptlv vnpdarlrlt reevfgpvvn lvrvadgeea |
| 421 | lrlaneteyg ltasvwtrdl sqalgysdrl qagtvwvnsh tlidpnlpfg gmkqsgtgrd |
| 481 | fgtdwldgwc etksvcvry |

SEQ ID NO: 24
SeqID: YP_002238842.1 GI: 206580270
Protein name: phenylacetaldehyde dehydrogenase
[Klebsiella pneumoniae 342]

| | |
|---|---|
| 1 | mstsqialla svqqfldrqh glyidgapca aqsenrltvw dpatgqaiat tadaspadvd |
| 61 | ravmsawraf vdrrwagrtp adrerillrf adlveqhgee laqletleqg ksiaisrafe |
| 121 | vgctlnwmry taglttkisg rtldvsipfp qgaryqawtk kepvgvvagi vpwnfplmig |
| 181 | mwkvmpalaa gcsivikpse ttpltllrva elatlagipd gvfnvvtgsg agcgaaltah |
| 241 | pqvakvsftg statgkqiar vaadrltrvt lelggknpai vlkdadpqwv ieglmtgsfl |
| 301 | nqgqvcaass riyieaplfd tlvsgfeqav kslqvgpgmq etaqinpvvs rahcdkvaay |
| 361 | leearqqkae lisgsagpda ggyyipptlv vnpdaglrlt reevfgpvvn lvrvadgeea |
| 421 | lrlandsdfg ltasvwtrdl tqalnytdrl qagtvwvnsh tlidanlpfg gmkqsgtgrd |
| 481 | fgpdwldgwc etksvcvry |

SEQ ID NO: 25
SeqID: YP_002919209.1 GI: 238894475
Protein name: phenylacetaldehyde dehydrogenase
[Klebsiella pneumoniae NTUH-K2044]

| | |
|---|---|
| 1 | mstsqialla svqqfldrqh glyidgapca aqsenrltvw dpatgqaiat tadaspadvd |
| 61 | ravmsawraf vdrrwvgrtp adrerillrf adlveqhgee laqletleqg ksiaisrafe |
| 121 | vgctlnwmry taglttkisg rtldvsipfp qgaryqawtk kepvgvvagi vpwnfplmig |
| 181 | mwkvmpalaa gcsivikpse ttpltllrva elatqagipd gvfnvvtgsg agcgaaltah |
| 241 | pqvakvsftg statgkqiar vaadrltrvt lelggknpai vlkdadpqwv ieglmtgsfl |
| 301 | nqgqvcaass riyieaplfd tlvsgfeqav kslqvgpgmq etaqinpvvs rahcdkvaay |
| 361 | leearqqkae lisgsagpda ggyyipptlv vnpdaglrls reevfgpvvn lvrvadgeea |
| 421 | lrlandsdfg ltasvwtrdl tqalnytdrl qagtvwvnsh tlidanlpfg gmkqsgtgrd |
| 481 | fgpdwldgwc etksvcvry |

SEQ ID NO: 26
SeqID: ZP_06549170.1 GI: 290509799
Protein name: phenylacetaldehyde dehydrogenase
[Klebsiella sp. 1_1_55]

| | |
|---|---|
| 1 | mstsqialla svqqfldrqh glyidgapca aqsenrltvw dpatgqaiat tadaspadvd |
| 61 | ravmsawraf vdrrwvgrtp adrerillrf adlveqhgee laqletleqg ksiaisrafe |
| 121 | vgctlnwmry taglttkisg rtldvsipfp qgaryqawtk kepvgvvagi vpwnfplmig |
| 181 | mwkvmpalaa gcsivikpse ttpltllrva elatlagipd gvfnvvtgsg agcgaaltah |
| 241 | pqvakvsftg statgkqiar vaadrltrvt lelggknpai vlkdadpqwv ieglmtgsfl |
| 301 | nqgqvcaass riyieaplfd tlvsgfeqav kslqvgpgmq etaqinpvvs rahcdkvaay |
| 361 | leearqqkae lisgsagpdt ggyyipptlv vnpdaglrlt reevfgpvvn lvrvadgeea |
| 421 | lrlandsdfg ltasvwtrdl tqalnytdrl qagtvwvnsh tlidanlpfg gmkqsgtgrd |
| 481 | fgpdwldgwc etksvcvry |

SEQ ID NO: 27
SeqID: ZP_06636953.1 GI: 293392633
Protein name: phenylacetaldehyde dehydrogenase
[Serratia odorifera DSM 4582]

| | |
|---|---|
| 1 | mpdntvtild svsqfldrqh glyidgqsqp sqaeqrlpvh npadgqqist tadasaedva |
| 61 | ravtsahraf tagvwaqrlp aererillry adlveqhaeq laqletleqg ksimisrdfe |
| 121 | vgctlnwmry taglatkitg qtldvsipmp pgaryqvytr kepigvvagi vpwnfplmig |
| 181 | iwkvmpalaa gcsivikpse ttpltllrma elaseagipp gvfnvvtgrg avcgkalteh |
| 241 | plvakvsftg stpvgkgiar aaadrltrvt lelggknpai vlqdaditqv veglmmgsfl |
| 301 | nqgqvcaass riyieapiyd nvvagfeqav kslsvgpgmd traqitplvs rshrdrvaaf |
| 361 | lddakakhae liagangpvg dgfyipptlv inpaanlnlt reevfgpvvn lirvadgeea |
| 421 | lrlandseyg ltaslwtrsl qaamaytpri qagtvwvnth tlidanmpfg gfkqsgsgrd |
| 481 | fgpdwldayt esksvciry |

SEQ ID NO: 28
SeqID: ZP_06191742.1 GI: 270263473
Protein name: hypothetical protein SOD_e00970
[Serratia odorifera 4Rx13]

| | |
|---|---|
| 1 | mpdntqavmd svsrfldrqh glyidgqwce ssaddrlavy npadgqqiss tadanaqdva |
| 61 | ravqsahkaf ttgawaqrlp aererillry adlveqhaee laqletleqg ksiniarafe |
| 121 | vgctlnwmry taglttkivg qtldvsipmp pgakyqvytr kepigvvagi vpwnfplmig |
| 181 | mwkvmpalaa gcsivikpse ttpltllrma elateagvpp gvfnvvtgrg tgcgkalteh |
| 241 | pliakvsftg stpvgksiar saadrltrvt lelggknpai vlkdadpqqv ieglmmgsfl |
| 301 | nqgqvcaass riyieapiyd rlvagfeqav kslsvgpgmd ataqinplvs rdhrnkvaay |
| 361 | lddarakhae lisgaagpda qgfyipptlv inpddklnlt reevfgpvvn lirvadaeea |
| 421 | lskandtdyg ltaslwttsl qqamaltpri qagtvwvnth tlidanmpfg gfkqsgsgrd |
| 481 | fgpdwldayt etksvciry |

SEQ ID NO: 29
SeqID: YP_001477217.1 GI: 157369228
Protein name: phenylacetaldehyde dehydrogenase
[Serratia proteamaculans 568]

| | |
|---|---|
| 1 | mpdntlavmd svsrfldrhh glyidgqwce ssaehrlavf npadgkqiss tadanaqdva |
| 61 | ravqsahqaf tsgvwaqrlp aererillry adlleqhtee laqletleqg ksiniarmfe |
| 121 | vgctlnwmry taglttkitg qtldvsipmp pgakyqvytr kepigvvagi vpwnfplmig |
| 181 | mwkvmpalaa gcsivikpse ttpltllrma elaseagipp gvfnvvtgkg tgcgkalteh |
| 241 | pliakvsftg stpvgkgiar aaadrltrvt lelggknpai vlkdadqqqv ieglmagsfl |
| 301 | nqgqvcaass riyieapiyd slvagfeqav ksltvgpgmd anaqinplvs sdhrnkvaay |
| 361 | lddarskhae lisgaagpds qgfyipptlv inpdeqlnla reevfgpvvn lirvadaeea |
| 421 | lskandtdyg ltaslwttsl qaamaytpri qagtvwvnth tlidanmpfg gfkqsgsgrd |
| 481 | fgpdwldayt esksvciry |

SEQ ID NO: 30
SeqID: YP_003188703.1 GI: 258543270
Protein name: aldehyde dehydrogenase
[Acetobacter pasteurianus IFO 3283-01]

| | |
|---|---|
| 1 | mdsllasvan fvsishelyi nggavpssgd arlpiydpst gmqiastvda taqdvdravt |
| 61 | safnsfksgi wkdmrpaere rillrladlv erdaeilaql etleqgksla israleagga |
| 121 | qtwiryvagl attitgktfd vsipfppdar ytsytrrtpv gvvagiipwn fplligvwkv |
| 181 | lpalaagcsv vakpaettpl tllylarlat eagvpdgvfn vvtgrgtvag sqlvnhplvs |
| 241 | kisftgstpv gkaiarscad slkrfslelg gknpaivldd adleqtvqgl mlasflnqgq |
| 301 | vcaacsriyv tdkmfeplrn altqaiqnmt vgagmnlqaq inpvvsaaqq kkilsyvqna |
| 361 | dteaeqviig qngpnaegyy vpptliinps peaacvteei fgpvltltrt sdgnealqla |
| 421 | ntssfglaas vwtqnlqaam tlpaqleagt vwvnshvmid pnmpfgglkq sgsgadfgsd |
| 481 | wldsftiqks icirh |

SEQ ID NO: 31
SeqID: ZP_07575384.1 GI: 307295548
Protein name: Aldehyde Dehydrogenase
[Sphingobium chlorophenolicum L-1]

| | |
|---|---|
| 1 | mtytgpftld ptaaaflgra palfidgrsv aadgrgclpv ydpssgtiia evadasapdv |
| 61 | dravcsahaa fvdgrwrnlr padrervllr ladllevrae afaqlesleq gksiniarmi |
| 121 | evgasidwir yaaglatkis grtfdlslpg gpthwtaytr repvgvvaai apwnfplmia |
| 181 | lwkvlpalas gcsivlkpse vtpltallla emaleagvpa gvfnvvtgsg avagralaeh |
| 241 | plvakisftg statgkaigh aaidgmkrft lelggknpal ilrdaklekv vpglmaggfl |
| 301 | nggqvcaavs riyveaplyd dlvaalsgai aavtvgpgld peaqlnplvs athsakvksy |
| 361 | lddadaagak ivrgaavpee gyyvspalil napaeaklvr eevfgpvlni srvadaeegl |
| 421 | rlandndlgl aaslwtqdid qamaltrrie agtiwvnshv fidpnmpfgg fkqsglgrdf |
| 481 | gmdwldgyte eksiciah |

SEQ ID NO: 32
SeqID: ZP_03698599.1 GI: 224825494
Protein name: Aldehyde Dehydrogenase
[Lutiella nitroferrum 2002]

| | |
|---|---|
| 1 | mdqnlvpvlp vvsaflrkeh gllvdgtsvq araggrievr npatgevias vadgdeqdve |
| 61 | alvqsahraf aggvwsglrp adrerillkf aevieahgee laqletlnqg ksihisraie |
| 121 | vgasveyvry magwatkitg etmdvsiavp qgtrytaytr repagvvaai vpwnfplmia |
| 181 | iwklipalaa gctivlkpst etpltalrlg elaleagipp gvvnvltgrg sragqalash |
| 241 | plvskisftg stdigktvah aavdnmtrfs lelggknpmv vladadvdka iqgvmggfl |
| 301 | nqgqvcaaas rlyihrskfd qivegvadtv rgmtlgsgld ltaqvnplvs skqqqsvcry |
| 361 | ldiaraegar vlagggksdr pgyfvqptvl tnvdhsktvv reeifgpvlv ampfdsveea |
| 421 | iqlandtpyg laaslwtndl savmnltpsi qagtvwvnsh vpldpnlpfg ghkqsgvgre |
| 481 | fgrtavesft elksvciah |

SEQ ID NO: 33
SeqID: ZP_02376853.1 GI: 167584465
Protein name: Betaine-aldehyde dehydrogenase
[Burkholderia ubonensis Bu]

| | |
|---|---|
| 1 | mtsstfvavs dtvrrfvard fglfidgdmq pahasarldv hdpatgerla tvadadehdv |
| 61 | eravasarra fdarvwsglr padrerillk ladlierdae tlaqletlnq gksihvarav |
| 121 | evgasveyvr ymagwatkit gqtldvsipf ppgarytayt rkepvgvvaa ivpwnfplmi |
| 181 | avwklvpala agctivlkps petpltalrl aelareagvp pgvfnvvtgg rvcgaalash |
| 241 | psiakisftg statgklvga aavqnmtrfs lelggknpiv mlddvdvtqa ldgvaagaff |
| 301 | nqgqvcaaas riyvhrskfa qladglagia qsmklgpgld taaqvnplvs ahhrdkvvqh |
| 361 | ieqargdght flaggtpadd lpgyfvkpav iadprpdsai vrdevfgpvv vvlpfddaad |
| 421 | avrlanaspy glaasiwsnd ltrvmnlvpq ieagtvwvnc hipldpsmpf ggykqsgigr |
| 481 | efgqhaidgf tetksvciah |

SEQ ID NO: 34
SeqID: YP_371691.1 GI: 78061783
Protein name: betaine-aldehyde dehydrogenase
[Burkholderia sp. 383]

| | |
|---|---|
| 1 | msttnfvavs dtvrtfvard fglfidgemq pahaaarldv ydpatgerla tvadaderdv |
| 61 | dravasakha fdtrvwsglr padrerillk ladlierdae tlaqletlnq gksihvsrai |
| 121 | evgasveyvr ymagwatkit gqtldvsipf ppgarytayt rkepvgvvaa ivpwnfplmi |
| 181 | avwklipala agctivlkps petpltalrl aelaleagvp pgvfnvvtgg rvcgaalash |
| 241 | psiakisftg statgklvga aavqnmtrfs lelggknpiv mlddidvaqa ldgvaagaff |
| 301 | nqgqvcaaas riyvhrskfa qladglagva qsmklgagld ttaqinplvs ahhrdkvvqh |
| 361 | iegarraglt flaggtpadd lpgyyvkpav iadphpdsai vrdevfgpvi vvvpfddaad |
| 421 | avrlanaspy glaasiwsnd lkrvmnlvpq ieagtvwvnc hipldpsmpf ggykqsgigr |
| 481 | efgqyaiegf tetksvciah |

SEQ ID NO: 35
SeqID: ZP_02909600.1 GI: 171320577
Protein name: Betaine-aldehyde dehydrogenase
[Burkholderia ambifaria MEX-5]

| | |
|---|---|
| 1 | mtiietpmst tnfvavsdtv rtfvardfgl fidgamqpah saarldvydp atgerlatva |
| 61 | dadahdvdra vasakhafdt rvwsglrpad rerillklad lieadaetla qletlnqgks |
| 121 | ihvsraievg asveyvryma gwatkitgqt ldvsipfppg arytaytrke pvgvvaaivp |
| 181 | wnfplmiavw klvpalaagc tivlkpspet pltalrlael areagvppgv fnvvtggrtc |
| 241 | gaalsshpsi akisftgsta tgklvgaaav qnmtrfslel ggknpivmld dvdvaqaldg |
| 301 | vaagaffnqg qvcaaasriy vhrskfaqla dglagvaqsm klgagldtta qinplvsahh |
| 361 | rdkvlqhieg arragltfla ggtpaddlpg yyvkpaviad phpdsaivrd evfgpvivvv |
| 421 | pfddaadavr lanaspygla asiwsndlkr vmnlvpriea gtvwvnchip ldpsmpfggy |
| 481 | kqsgigrefg qyaiegftet ksvciah |

SEQ ID NO: 36
SeqID: YP_001811358.1 GI: 172063707
Protein name: betaine-aldehyde dehydrogenase
[Burkholderia ambifaria MC40-6]

| | |
|---|---|
| 1 | msttnfvavs dtvrtfvard fglfiegamq pahsaarldv ydpatgerla tvadadahdv |
| 61 | dravasakha fdtrvwsglr paererillr ladlieadae tlaqletlnq gksihvsrai |
| 121 | evgasveyvr ymagwatkit gqtldvsipf ppgarytayt rkepvgvvaa ivpwnfplmi |
| 181 | avwklipala agctivlkps petpltalrl aelardagvp pgafnvvtgg rtcgaalssh |
| 241 | psiakisftg statgklvga aavqnmtrfs lelggknpiv mlddvdvaqa ldgvaagaff |
| 301 | nqgqvcaaas riyvhrskfa qladglagia qsmklgagld ttaqinplvs ahhrdkvvqh |
| 361 | iegarraglt flaggtpadd lpgyyvkpav iadphpdsai vrdevfgpvi vvvpfddaad |
| 421 | avrlanaspy glaasiwsnd lkrvmnlvpr ieagtvwvnc hipldpsmpf ggykqsgigr |
| 481 | efgqyavegf tetksvciah |

SEQ ID NO: 37
SeqID: YP_002234586.1 GI: 206563823
Protein name: phenylacetaldehyde dehydrogenase
[Burkholderia cenocepacia J2315]

| | |
|---|---|
| 1 | msntsfvavs dtvrtfvard fglfidgamq pahaaarldv ydpatgerla tvadadahdv |
| 61 | dravasarha fdarvwcglr padrerillk ladlieadae tlaqletlnq gksihvsrai |
| 121 | evgasveyvr ymagwatkit gqtldvsipf ppgarytayt rkepvgvvaa ivpwnfplmi |
| 181 | avwklvpala agctivlkps petpltalrl aelareagvp pgvfnvvtgg rvcgaalash |
| 241 | psiakisftg statgklvga aavqnmtrfs lelggknpiv mlddvdvaqa ldgvaagaff |
| 301 | nqgqvcaaas riyvhrskfa qladglagva rsmklgagld ttaqinplvs ahhrdkvvqh |
| 361 | iegarraglt flaggtpadd lpgyyvkpav iadphpdsai vrdevfgpvi vvvpfddaad |
| 421 | avrlanaspy glaasvwsnd lkrvmnlvpq ieagtvwvnc hipldpsmpf ggykqsgigr |
| 481 | efgqyaiegf tetksvciah |

SEQ ID NO: 38
SeqID: ZP_02894048.1 GI: 170703247
Protein name: Betaine-aldehyde dehydrogenase
[Burkholderia ambifaria IOP40-10]

| | |
|---|---|
| 1 | msttnfvavs dtvrtfvard fglfidgamq pahsaarldv ydpatgerla tvadadahdv |
| 61 | dravasakha fdtrvwsglr paererillk laelieadae tlaqletlnq gksihvsrai |
| 121 | evgasveyvr ymagwatkit gqtldvsipf ppgarytayt rkepvgvvaa ivpwnfplmi |
| 181 | avwklipala agctivlkps petpltalrl aelareagvp pgafnvvtgg racgaalssh |
| 241 | psiakisftg statgklvga aavqnmtrfs lelggknpiv mlddvdvaqa ldgvaagaff |
| 301 | nqgqvcaaas rlyvhrskfa qladglagva qsmklgagld ttaqinplvs ahhrdkvvqh |
| 361 | iegarraglt flaggtpadd lpgyyvkpav iadphpdsai vrdevfgpvi vvvpfddaad |
| 421 | avrlanaspy glaasiwsnd lkrvmnlvpr ieagtvwvnc hipldpsmpf ggykqsgigr |
| 481 | efgqyavegf tetksvciah |

SEQ ID NO: 39
SeqID: ZP_04943579.1 GI: 254250259
Protein name: NAD-dependent aldehyde dehydrogenase
[Burkholderia cenocepacia PC184]

| | |
|---|---|
| 1 | mvrrpraspa rgpattinet pmsntnfvav sdtvrtfvar dfglfidgam qpshaaarvd |
| 61 | vydpatgerl atvadadahd vdravasakh afdtrvwsgl rpadrerill kladlieada |
| 121 | etlaqletln qgksihvsra vevgasveyv rymagwatki tgqtldvsip fppgarytay |
| 181 | trkepvgvva aivpwnfplm iavwklipal aagctivlkp spetpltalr laelareagv |
| 241 | ppgvfnvvtg grvcgaalas hpsiakisft gstatgklvg aaavqnmtrf slelggknpi |
| 301 | vmlddvdvaq aldgvaagaf fnqgqvcaaa sriyvhrskf aqladglagv arsmklgagl |
| 361 | dttaqinplv saqhrdkvvq hiegarragl tflaggtpad dlpgyyvkpa viadphpdsa |
| 421 | ivrdevfgpv ivvvpfddaa davrlanasp yglaasiwsn dlkrvmnlvp qieagtvwvn |
| 481 | chipldpsmp fggykqsgig refgqyaieg ftetksvcia h |

SEQ ID NO: 40
SeqID: YP_776051.1 GI: 115358913
Protein name: betaine-aldehyde dehydrogenase
[Burkholderia ambifaria AMMD].

| | |
|---|---|
| 1 | msttnfvavs dtvrtfvard fglfidgamq pahsaarfdv ydpatgerla tvadadahdv |
| 61 | dravasakha fdtrvwsglr padrerillk ladlierdae tlaqletlnq gksihvsrai |
| 121 | evgasveyvr ymagwatkit gqtldvsipf ppgarytayt rkepvgvvaa ivpwnfplmi |
| 181 | avwklipala agctivlkps petpltalrl aelareagvp pgafnvvtgg rtcgaalssh |
| 241 | psiakisftg statgklvga aavqnmtrfs lelggknpiv mlddvdvaqa ldgvaagaff |
| 301 | nqgqvcaaas rlyvhrskfa qladglagva qsmklgagld ttaqinplvs ahhrdkvvqh |
| 361 | iegarraglt flaggtpadd lpgyyvkpav iadphpdsai vrdevfgpvi vvvpfddaad |
| 421 | avrlanaspy glaasiwsnd lkrvmnlvpq ieagtvwvnc hipldpsmpf ggykqsgigr |
| 481 | efgqyaiegf tetksvciah |

SEQ ID NO: 41
SeqID: ZP_03569003.1 GI: 221195956
Protein name: phenylacetaldehyde dehydrogenase
[Burkholderia multivorans CGD2M]

| | |
|---|---|
| 1 | msttnfvavs dtvrtflard fglfidgamq pahaaarldv ydpatgerla tvadadeydv |
| 61 | dravasakra fdarvwrglr padrerillk laelmerdae tlaqletlnq gksihvsrav |
| 121 | evgasveyvr ymagwatkit gqtldvsipf ppgarytayt rkepvgvvaa ivpwnfplmi |
| 181 | atwklipala agctvvlkps petpltalrl aelaleagvp pgvfnvvtgg rtcgaalarh |
| 241 | psiakisftg stetgklvga aavqnmtrfs lelggknpiv mledvdvgqa ldgvaagaff |
| 301 | nqgqvcaaas riyvhrskfa rladglaeha ramtlgpgld taaqinplvs aqhrdkvvrh |
| 361 | ieqarrdgvt flaggtrvde lpgyyvrpav iadpradsai vrdevfgpvi vvlpfddaad |
| 421 | avrlanaspy glaasiwsnd ltrvmnlvpq ieagtvwvnc hipldpsmpf ggykqsgigr |
| 481 | efgqyaiegf tetksvciah |

SEQ ID NO: 42
SeqID: ZP_06296391.1 GI: 282887812
Protein name: Aldehyde Dehydrogenase
[Burkholderia sp. CCGE1001]

| | |
|---|---|
| 1 | mssdakvalc davrafvgre hglfidgaac lahsprrsnv fdpatgnvlt tvpdadatdv |
| 61 | dravtsarva fearvwrglr padrerillr fadvleahae elaqletlnq gksinvarai |
| 121 | dvgatleyvr ymagwatkiv getldvsipf ppgarytaft rhepvgvvag ivpwnfpmmi |
| 181 | avwklvpala agcsvvikps petpltalrl aelaveagip agafnvvtgg aecgaalaah |
| 241 | pginkisftg stptgkrvgi aavqnmtrfs lelggknpav mladidveqa vqgalaggll |
| 301 | nqgqvcaavs riyvhrskya kvvegladsv samtmgpgld psahinplvs ahhrarveeh |
| 361 | lvraraeglr faaggarvee pgyyvrpavi advppeaaiv rdevfgpvla lapfddvaha |
| 421 | lrlandspyg laaslwtndl raamnlvpqi eagtvwvnch vpldpgmpfg gykqsgigre |
| 481 | fgrhaiegft etksvciah |

SEQ ID NO: 43
SeqID: YP_623452.1 GI: 107025941
Protein name: betaine-aldehyde dehydrogenase
[Burkholderia cenocepacia AU 1054]

| | |
|---|---|
| 1 | msntnfvavs dtvrtfvard fglfidgamq tahaaarvdv ydpatgerla tvadadahdv |
| 61 | dravasakha fdtrvwsglr padrerillk ladlieadae tlaqletlnq gksihvsrav |
| 121 | evgasveyvr ymagwatkit gqtldvsipf ppgarytayt rkepvgvvaa ivpwnfplmi |
| 181 | avwklipala agctivlkps petpltalrl aelareagvp pgvfnvvtgg rvcgaalash |
| 241 | psiakisftg statgklvga aavqnmtrfs lelggknpiv mlddvdvaqa ldgvaagaff |
| 301 | nqgqvcaaas riyvhrskfa qladglagva qsmklgagld ttaqinplvs aqhrdkvvqh |
| 361 | iegarraglt flaggspadd lpgyyvkpav iadphpdsai vhdevfgpvi vvvpfddaad |
| 421 | avrlanaspy glaasiwsnd ltrvmnlvpq ieagtvwvnc hipldpsmpf ggykqsgigr |
| 481 | efgqyaiegf tetksvciah |

SEQ ID NO: 44
SeqID: ZP_03582658.1 GI: 221209677
Protein name: phenylacetaldehyde dehydrogenase
[Burkholderia multivorans CGD1]

| | |
|---|---|
| 1 | msttnfvavs dtvrtflard fglfidgamq rahaaarldv ydpatgerla tvadadehdv |
| 61 | dravasakra fdarvwrglr padrerillk ladlierdae tlaqletlnq gksihvsrav |
| 121 | evgasveyvr ymagwatkit gqtldvsipf ppgarytayt rkepvgvvaa ivpwnfplmi |
| 181 | atwklipala agctvvlkps petpltalrl aelaleagvp pgvfnvvtgg rtcgaalarh |
| 241 | psiakisftg stetgklvga aavqnmtrfs lelggknpiv mlddvdvgqa lggvaagaff |
| 301 | nqgqvcaaas riyvhrskfa qladglaeha ramtlgpgld ttaqinplvs aqhrdkvvrh |
| 361 | ieqarrdgvt flaggtrvde lpgyyvrpav iadpradsai vrdevfgpvi vvlpfddaad |
| 421 | avrlanaspy glaasiwsnd ltrvmnlvpq ieagtvwvnc hipldpsmpf ggykqsgigr |
| 481 | efgqyaiegf tetksvciah |

SEQ ID NO: 45
SeqID: ZP_04947622.1 GI: 254254305
Protein name: NAD-dependent aldehyde dehydrogenase
[Burkholderia dolosa AUO158]

| | |
|---|---|
| 1 | mtssptfvav sdtvrafvar dfglfidgdm qrahaaarld vydpatgerl atvadadahd |
| 61 | vdravasaks afdarvwsgl rpadrerill kladlierda etlaqletln qgksihvsra |
| 121 | vevgasveya rymagwatki tgqtldvsip fppgtrytay trkepvgvva aivpwnfplm |
| 181 | iavwklipal aagctivlkp spetpltalr laelareagv ppgafnvvtg grtcgaalas |
| 241 | hpsiakisft gstatgklvg aaavqnmtrf slelggknpi vmlddvdvaq aldgvaagaf |
| 301 | fnqgqvcaaa sriyvhrsrf sqladglagv anamklgpgl dmaaqvnplv sahhrdkvva |
| 361 | hierarrdgl tflaggtlad dlsgyfvkpa viadphpdsa ivrdevfgpv ivvvpfddaa |
| 421 | davrlanasp yglaasiwsn dlkrvmnlvp qieagtvwvn chipldpsmp fggykqsgig |
| 481 | refgqyaieg ftetksvcia h |

SEQ ID NO: 46
SeqID: ZP_02365539.1 GI: 167572665
Protein name: phenylacetaldehyde dehydrogenase
[Burkholderia oklahomensis C6786]

| | |
|---|---|
| 1 | mtqtdfvtvt dtvraftere fgifidgamr aahsprrldv fdpatgerla rvpdadahdv |
| 61 | daavasakra fdarawsglr padrerillk ladaleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtgg recgaalash |
| 241 | psirkisftg statgklvga aavqnmtrfs lelggknpiv mledvdvaqa legvaagaff |
| 301 | nqgqvcaaas riyvhrsrfr rladglagva asmrlgpgld paaqinplvs ahhrdkvveh |
| 361 | verarrdglt flaggapadd lpgyfvrpav iadathdsai vrdevfgpvv vvlpfddpae |
| 421 | avrlanaspy glaaslwsnd lkavmdlvtq ieagtvwvnc hipldpsmpf ggykqsgigr |
| 481 | efgqyaidgf tetksvciah |

SEQ ID NO: 47
SeqID: YP_001075236.1 GI: 126455560
Protein name phenylacetaldehyde dehydrogenase
[Burkholderia pseudomallei 1106a]

| | |
|---|---|
| 1 | mtqtdlvava dtvraftere fgvfidgamr aahsprrldv fdpatgarls rvpdadahdv |
| 61 | daavasaqra fdarawsglr paererillk ladvleahae elaqletlnq gksilvsrsv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtga racgaalash |
| 241 | pavrkisftg statgklvga aavqnmtrfs lelggknpiv mledvdvdaa lggvaagaff |
| 301 | nqgqvcaaas riyvhrsrfr rlaeglagva samrlgpgld paaqinplvs ahhrdtvarh |
| 361 | idaarreglt flaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 48
SeqID: ZP_04522864.1 GI: 237510149
Protein name phenylacetaldehyde dehydrogenase
[Burkholderia pseudomallei MSHR346]

| | |
|---|---|
| 1 | mraaraatgt srmihpqhet hmtqtdlvav adtvrafter efgvfidgam raahsprrld |
| 61 | vfdpatgarl srvpdadahd vdaavasaqr afdarawsgl rpaererill kladvleaha |
| 121 | eelaqletln qgksilvsrg vevgatieyv rymagwatki tgqtldvsip fppgarytay |
| 181 | trkepvgvva aivpwnfplm iavwklvpal aagctvvlkp spetpltalr laelaleagv |
| 241 | pagvfnvvtg aracgaalas hpavrkisft gstatgklvg aaavqnmtrf slelggknpi |
| 301 | vmledvdvda alggvaagaf fnqgqvcaaa sriyvhrsrf rrlaeglagv asamrlgpgl |
| 361 | dpaaqinplv sahhrdtvar hidaarregl tflaggtrad dlpgyfvrpa viadaahdsa |
| 421 | ivrdevfgpv vvvlpfddpa eavrlanasp yglaaslwsn dlkavmdlvp qieagtvwvn |
| 481 | chipldpsmp fggykqsgig refgqyaieg ftetksvcia h |

SEQ ID NO: 49
SeqID: ZP_02509455.1 GI: 167922364
Protein name phenylacetaldehyde dehydrogenase
[Burkholderia pseudomallei BCC215]

| | |
|---|---|
| 1 | mtqtdlvava dtvraftere fgvfidgamr aahsprrldv fdpatgarls rvpdadahdv |
| 61 | daavasaqra fdarawsglr paererillk ladvleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtga racgaalash |
| 241 | pavhkisftg statgklvga aavqnmtrfs lelggknpiv mledvdvdaa lggvaagaff |
| 301 | nqgqvcaaas riyvhrsrfr rlaeglagva samrlgpgld paaqinplvs ahhrdtvarh |
| 361 | idaarreglt flaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 50
SeqID: YP_110877.1 GI: 53721892
Protein name phenylacetaldehyde dehydrogenase
[Burkholderia pseudomallei K96243]

| | |
|---|---|
| 1 | mtqtdlvava dtvraftere fgvfidgamr aahsprrldv fdpatgarls rvpdadahdv |
| 61 | daavasaqra fdarawsglr paererillk ladvleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtga racgaalash |
| 241 | pavrkisftg statgklvga aavqnmtrfs lelggknpiv mledvdvdaa lggvaagaff |
| 301 | nqgqvcaaas riyvhrsrfr rlaeglagva samrlgpgld paaqinplvs ahhrdtvarh |
| 361 | idaarreglt flaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 51
SeqID: YP_105981.1 GI: 53717194
Protein name phenylacetaldehyde dehydrogenase
[Burkholderia mallei ATCC 23344]

| | |
|---|---|
| 1 | mtqtdlvava dtvraftere fgvfidgamr aahsprrldv fdpatgarls rvpdadahdv |
| 61 | daavasaqra fdarawsglr paererillk ladvleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtga racgaalash |
| 241 | pavrkisftg statgklvga aavqnmtrfs lelggknpiv mledvdvdaa lggvaagaff |
| 301 | nqgqvcaaas riyvhrsrfr rlaeglagva samrlgpgld paaqinplvs ahhrdtvarh |
| 361 | idaarreglt ffaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 52
SeqID: ZP_04880788.1 GI: 254174126
Protein name phenylacetaldehyde phenylacetaldehyde dehydrogenase
[Burkholderia mallei ATCC 10399]

| | |
|---|---|
| 1 | miqpqhethm tqtdlvavad tvrafteref gvfidgamra ahsprrldvf dpatgarlsr |
| 61 | vpdadahdvd aavasaqraf darawsglrp aererillkl advleahaee laqletlnqg |
| 121 | ksilvsrgve vgatieyvry magwatkitg qtldvsipfp pgarytaytr kepvgvvaai |
| 181 | vpwnfplmia vwklvpalaa gctvvlkpsp etpltalrla elaleagvpa gvfnvvtgar |
| 241 | acgaalashp avrkisftgs tatgklvgaa avqnmtrfsl elggknpivm ledvdvdaal |
| 301 | ggvaagaffn qgqvcaaasr iyvhrsrfrr laeglagvas amrlgpgldp aaqinplvsa |
| 361 | hhrdtvarhi daarregltf faggtraddl pgyfvrpavi adaahdsaiv rdevfgpvvv |
| 421 | vlpfddpaea vrlanaspyg laaslwsndl kavmdlvpqi eagtvwvnch ipldpsmpfg |
| 481 | gykqsgigre fgqyaiegft etksvciah |

SEQ ID NO: 53
SeqID: ZP_04889911.1 GI: 254183319
Protein name phenylacetaldehyde dehydrogenase
[Burkholderia pseudomallei 1655]

| | |
|---|---|
| 1 | mtqtdlvava dtvraftere fgvfidgamr aahsprrldv fdpatgarls rvpdadahdv |
| 61 | daavasaqra fdarawsglr paererillk ladvleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtga recgaalash |
| 241 | pavrkisftg statgklvga aavqnmtrfs lelggknpiv mledvdvdaa lggvaagaff |
| 301 | nqgqvcaaas riyvhrsrfr rlaeglagva samrlgpgld paaqinplvs ahhrdtvarh |
| 361 | idaarreglt flaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 54
SeqID: ZP_01766119.1 GI: 134279406
Protein name phenylacetaldehyde dehydrogenase
[Burkholderia pseudomallei 305]

| | |
|---|---|
| 1 | mihpqhethm tqtdlvavad tvrafteref gvfidgamra ahsprrldvf dpatgarlsr |
| 61 | vpdadahdvd aavasaqraf darawsglrp aererillkl advleahaee laqletlnqg |
| 121 | ksilvsrgve vgatieyvry magwatkitg qtldvsipfp pgahytaytr kepvgvvaai |
| 181 | vpwnfplmia vwklvpalaa gctvvlkpsp etpltalrla elaleagvpa gvfnvvtgar |
| 241 | acgaalashp avrkisftgs tatgklvgaa avqnmtrfsl elggknpivm ledvdvdaal |
| 301 | ggvaagaffn qgqvcaaasr iyvhrsrfrr laeglagvas amrlgpgldp aaqinplvsa |
| 361 | hhrdtvarhi daarregltf laggtraddl pgyfvrpavi adaahdsaiv rdevfgpvvv |
| 421 | vlpfddpaea vrlanaspyg laaslwsndl kavmdlvpqi eagtvwvnch ipldpsmpfg |
| 481 | gykqsgigre fgqyaiegft etksvciah |

SEQ ID NO: 55
SeqID: YP_001062271.1 GI: 126443343
Protein name phenylacetaldehyde dehydrogenase
[Burkholderia pseudomallei 668]

| | |
|---|---|
| 1 | mtqtdlvava dtvraftere fgvfidgamr aahsprrldv fdpatgarls rvpdadahdv |
| 61 | daavasaqra fdarawsglr paererillk ladvleahae elaqletlnq gksilvsrgv |
| 121 | evgatieyvr ymagwatkit gqtldvsipf ppdarytayt rkepvgvvaa ivpwnfplmi |
| 181 | avwklvpala agctvvlkps petpltalrl aelaleagvp agvfnvvtga racgaalash |
| 241 | pavrkisftg statgklvga aavqnmtrfs lelggknpiv mledvdvdaa lggvaagaff |
| 301 | nqgqvcaaas riyvhrsrfr rlaeglagva samrlgpgld paaqinplvs ahhrdtvarh |
| 361 | idaarreglt flaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 56
SeqID: YP_439732.1 GI: 83718371
Protein name phenylacetaldehyde dehydrogenase
[Burkholderia thailandensis E264]

| | |
|---|---|
| 1 | mkrtrrarrr tsarsarsda pyspaqpgrv paappnlpic anpprsrvvt lvrpiraams |
| 61 | gagrpiepqh ethmtqtdlv avadtvraft krefgifidg amraahsprr ldvydpatge |
| 121 | rlsrvpdada hdvdaavasa krafdarvws glrpadrerv llkladvlea haeelaqlet |
| 181 | lnqgksilvs rgvevgatie yvrymagwat kitgqtldvs ipfppgaryt aytrkepvgv |
| 241 | vaaivpwnfp lmiavwklvp alaagctvvl kpspetplta lrlaelalea gvpagvfnvv |
| 301 | tggrecgaal aghpsvrkis ftgstatgkl vgaaavqnmt rfslelggkn pivmlddvdv |
| 361 | daalggvaag affnqgqvca aasriyvhrs kfrrlaqgla dvaaamrlgp gldpaaqinp |
| 421 | lvsahhrdkv vqhievarre gltfltggtr addlpgyfvr paviadaahd saivrdevfg |
| 481 | pvvvvlpfdd paeavrlana spyglaaslw sndlkavmdl vpqieagtvw vnchipldps |
| 541 | mpfggykqsg igrefgqyai egftetksvc iah |

SEQ ID NO: 57
SeqID: ZP_02384933.1 GI: 167616298
Protein name phenylacetaldehyde dehydrogenase
[Burkholderia thailandensis Bt4]

| | |
|---|---|
| 1 | mtqtdlvava dtvraftkre fgifidgamr aahsprrldv ydpatgerls rvpdadahdv |
| 61 | daavasakra fdarvwsglr padrervllk ladvleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtgg recgaalagh |
| 241 | psvrkisftg statgklvga aavqnmtrfs lelggknpiv mlddvdvdaa lggvaagaff |
| 301 | nqgqvcaaas riyvhrskfr rlaqgladva aamrlgpgld paaqinplvs ahhrdkvvqh |
| 361 | ievarreglt fltggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 58
SeqID: ZP_02371041.1 GI: 167578167
Protein name phenylacetaldehyde dehydrogenase
[Burkholderia thailandensis TXDOH]

| | |
|---|---|
| 1 | mtqtdlvtva dtvraftere fgifidgamr aahsprrldv ydpatgerls rvpdadahdv |
| 61 | daavasakra fdarvwsglr padrervllk ladvleahae elaqletlnq gksilvsrgv |
| 121 | evgatieyvr ymagwatkit gqtldvsipf ppgarytayt rkepvgvvaa ivpwnfplmi |
| 181 | avwklvpala agctvvlkps petpltalrl aelaleagvp agvfnvvtgg recgaalsgh |
| 241 | psvrkisftg statgklvga aavqnmtrfs lelggknpiv mlddvdvdaa lggvaagaff |
| 301 | nqgqvcaaas riyvhrskfr rlaqgladva aamrlgpgld paaqinplvs ahhrdkvvqh |
| 361 | ievarreglt flaggtradd lpgyfvrpav iadaahdsai vrdevfgpvv vvlpfddpae |
| 421 | avrlanaspy glaaslwsnd lkavmdlvpq ieagtvwvnc hipldpsmpf ggykqsgigr |
| 481 | efgqyaiegf tetksvciah |

SEQ ID NO: 59
SeqID: YP_001895626.1 GI: 187923984
Protein name Aldehyde dehydrogenase (NAD(+))
[Burkholderia phytofirmans PsJN]

| | |
|---|---|
| 1 | mtehklaatl phtmfingek tgsaagktfp vfnpataeei aqipdasead idhavrtska |
| 61 | afesdawrrm ppavreglll kladlverhs delatletln qgkligfskm levagsvqwl |
| 121 | rymagwatki egstfdlsip fppgtrynas tkrvpagvva aivpwnfpll mavwkiapal |
| 181 | acgctvvlkp aeetpltair laelaheagf pagvfnvvtg rgetagaalv rhplvkkvtf |
| 241 | tgstevgrii grqcaddlkr aslelggksp vivlddcdpr kaiegaagai ffnhgqvcta |
| 301 | gsrlyvarsi ydevvqgiaa vadgitlgsg fdaatqmgpm vsarhrdkva gmiaqgkdeg |
| 361 | geivsrdarv eregyfvrpt vianrackpl avvkeevfgp vlvampyddl devlaganas |
| 421 | eyglgasvwt nqldkalrlv dgieagtvwv nthnmvdpam pfggfkasgi grehgksiie |
| 481 | sytesksvci ay |

SEQ ID NO: 60
SeqID: ZP_02406444.1 GI: 167723208
Protein name phenylacetaldehyde dehydrogenase
[Burkholderia pseudomallei DM98]

| | |
|---|---|
| 1 | rafgarawsg lrpaereril lkladvleah aeelaqletl nqgksilvsr gvevgatiey |
| 61 | vrymagwatk itgqtldvsi pfppgaryta ytrkepvgvv aaivpwnfpl miavwklvpa |
| 121 | laagctvvlk pspetpltal rlaelaleag vpagvfnvvt garacgaala shpavrkisf |
| 181 | tgstatgklv gaaavqnmtr fslelggknp ivmledvdvd aalggvaaga ffnqgqvcaa |
| 241 | asriyvhrsr frrlaeglag vasamrlgpg ldpaaqinpl vsahhrdtva rhidaarreg |
| 301 | ltflaggtra ddlpgyfvrp aviadaahds aivrdevfgp vvvvlpfddp aeavrlanas |
| 361 | pyglaaslws ndlkavmdlv pqieagtvwv nchipldpsm pfggykqsgi grefgqyaie |
| 421 | gftetksvci ah |

SEQ ID NO: 61
SeqID: Z5_02461778.1 GI: 167834895
Protein name phenylacetaldehyde dehydrogenase
[Burkholderia thailandensis MSMB43]

| | |
|---|---|
| 1 | mnlaalstqh qrqsgflarr qfgnwldgsa aeprsgrylp vvdpatemti aevaasdard |
| 61 | vdaavaaarr afdsgdwprm rpasrekllh rladrierha delaaletle tgklvgvara |
| 121 | idvlggaeyv ryvagwatkl egstldtsia apagteyfay trreavgvvg aivpwnfpla |
| 181 | ialwkiatal acgctvvlkp seetpltalr lgelaqeagl pdgvlnvvtg tgaeagaalv |
| 241 | ahpgvdkitf tgsvgvgrai ghaavdrmar ftlelggksp livfddadpd vaargaaqgi |
| 301 | ffnqgqvcta gsrvyvqkrl feqvvagiaa aaesmkigsg fdpntqigpl vskrhfervl |
| 361 | shvdaakeeg atlvtggtra ldggyfvkpt vfvdaapsmr ivreevfgpv vtvtpfdtld |
| 421 | davrlandtd fglaasvwsq nlsrvhrvvp rlkagivwvn thnmldsnlp fggfkqsgyg |
| 481 | relgraaleq ftelksvcia h |

SEQ ID NO: 62
SeqID: YP_003605161.1 GI: 295676637
Protein name Betaine-aldehyde dehydrogenase
[Burkholderia sp. CCGE1002]

| | |
|---|---|
| 1 | mkieallanl rtqmivggra vdarsgktfa vydpasgrei aqvpdgdaed vaaavaaaks |
| 61 | afesnewrrm ppaarehlll kladlveqhg delaaletln qgkllgfsrm levggsaqwl |
| 121 | rymagwatki egstvdlsls fppgvqyras tqrvpagvva aivpwnfpll mavwkiapal |
| 181 | acgctvvlkp aeetpltair laelaleagl pagvlnvvtg rgetagaalv rhpdvdkvtf |
| 241 | tgstevgrli gaqcgrdirr aslelggksp vivlddcdpr kaiegaagai ffnhgqvcta |
| 301 | gsrlyvprsm yaqivegvaq vanslvlgsg fdektqmgpl vsarhrdkvv gmiaegraqg |
| 361 | geiiaggsar dgdgyfvrpt vvanearrpl slvneevfgp vlvampyddl eeaisaanss |
| 421 | eyglgasvwt nqldkalrvv drmeagtvwv nshnmvdpal pfggfkssgv grehgraiid |
| 481 | aytetksvcf ay |

SEQ ID NO: 63
SeqID: ZP_06686890.1 GI: 293604485
Protein name phenylacetaldehyde dehydrogenase
[Achromobacter piechaudii ATCC 43553]

| | |
|---|---|
| 1 | mhgptsqram pllfwlhtfr wsfpmssars saadnaaadr pelaairefm lidgkpvheg |
| 61 | qgqpvpvhdp atgrviahqp dagpaqvdla vqaarrafds gtwrdmlpag rerlllklad |
| 121 | lieqhgvela rletlnngkl lgiaqglevg asaqwlryma gwatkitgdt lslsipfppg |
| 181 | thyhaytlaq pvgvvgaiip wnfpllmavw kiapalaagc tvvlkpaeet pltalrlael |
| 241 | vmqagfppgv vnvitgrget agaalvahpg idkiaftgst evgkligrra mddmkrvsle |
| 301 | lggkspvivl ddcdvdcavq gaaaaiffnq gqvctagsrl yvqrglyakv vqgladvass |
| 361 | mtlgsgfdpa tqigplissq hqqrvldyig igraeggrvl tggaagegdg yfvrptvfad |
| 421 | vpqhgriaqe eifgpvvvaq pfdslddavr landsafglg asiwsndltr vqrliprida |
| 481 | gtvwvnthnm ldpnmpfggf kqsgvgrehg kavlemylek ksvcmay |

SEQ ID NO: 64
SeqID: YP_003469437.1 GI: 290476532
Protein name phenylacetaldehyde dehydrogenase
[Xenorhabdus bovienii SS-2004]

| | |
|---|---|
| 1 | mseitllkpv tdflqrphgn yinglslpgq gnrtfsvvnp asdemiakvn qgeeaeinla |
| 61 | meaaskafhg swaqtspmer gkclnrladl lqkhgeelaq leslcsgkpi qlarmldvga |
| 121 | sadylryfag wsskisgetl nvslpslkge kytaftrrep igvvvgiipw nfsimiaiwk |
| 181 | lgaalacgct lvlkpseytp ltmlrvaela keagipdgvi nvvngsgary gsallahphc |
| 241 | akvtftgsvp tgtiigksai eyglsratle lggknaaafl sdmsvekive gvleagylnq |
| 301 | gqicaaaerf yipsvhmdav lkllserlsa mkigspldes temgplanke hyekilslfd |
| 361 | karqdgseiv ygghalegag ffvtptiira ksaedtlmqe etfgpigtfl syddeeeliv |
| 421 | mmnatpfgls aslwtndlsk amrmipriqv gtlwinmhtf ldpalpfggv kssgigrefg |
| 481 | safiehytel ksvmvry |

SEQ ID NO: 65
SeqID: ZP_03317190.1 GI: 212709062
Protein name hypothetical protein PROVALCAL_00094
[Providencia alcalifaciens DSM 30120]

| | |
|---|---|
| 1 | mseltllpev reflkrqhgh finglpvsgk geayfdvvnp ateqviakvk egtreevdia |
| 61 | mnvayaafkg swanttpmer gnclnrladl lekhleelaq letlssgkti qlsrflevgs |
| 121 | aaqflryfag watkisgetl nvslpsfnge kysaftqrep vgvvagiipw nfsimisiwk |
| 181 | laaaltcgct ivlkpseftp ltmlrvaela keagipdgvi nivngggrev gpalishelc |
| 241 | skvtftgsvp tglavgrsam egkltrvtle lggkngaafl adlsvdkivs giieagylnq |
| 301 | gqicaaaerf yvpstlmdav leelkarlsa mkigsplded tqmgplanka hynkilslfe |
| 361 | karqdgseii yggqpiagag yfvpptiira kspddvlmke etfgpigtfl ayddeeelik |
| 421 | mmnstpfgla asvwtndlsk amrmvsqiea gtvwvnmhtf ldpavpfggi kssgigrefg |
| 481 | safiehytel ksvmvry |

SEQ ID NO: 66
SeqID: YP_440610.1 GI: 83719867
Protein name phenylacetaldehyde dehydrogenase
[Burkholderia thailandensis E264]

| | |
|---|---|
| 1 | mnlaalstqh qrqsgflarr qfgnwidgaa aeprsgrylp vvdpatemti aevaasdard |
| 61 | vdaavaaarr afdsgdwprm rpasrekllh qladrierya delaaletle tgklvglara |
| 121 | idvlggaeyv rymagwatkl egstldtsia vpagteyfay trreavgvvg aivpwnfpla |
| 181 | ialwkvatal acgctvvlkp seetpltalr lgelaqeagl pdgvlnvvtg tgadtgaalv |
| 241 | ahpgvdkitf tgsvgvgkai ghaavdrmar ftlelggksp livfddadpd vaargaaqgi |
| 301 | ffnqgqvcta gsrvyvhksl fervvagiaa aaesmkigsg fdpntqigpl vskrhfervl |
| 361 | ghidaakeeg atlvtggtra ldggyfvkpt vfvdaapsmr ivreevfgpv vtvtpfdtvd |
| 421 | davrlandsd fglaasvwsq nlsrvhrvvp rlkagivwvn thnmldnnlp fggvkqsgyg |
| 481 | relgraaleq ftelksvcia h |

SEQ ID NO: 67
SeqID: ZP_02372174.1 GI: 167579300
Protein name phenylacetaldehyde dehydrogenase
[Burkholderia thailandensis TXDOH]

| | |
|---|---|
| 1 | mnlaalstqh qrqsgflarr qfgnwidgaa aeprsgrylp vvdpatemti aevaasdard |
| 61 | vdaavaaarr afdsgdwprm rpasrekllh rladrierya delaaletle tgklvgvara |
| 121 | idvlggaeyv rymagwatkl egstldtsia vpadteyfay trreavgvvg aivpwnfpla |
| 181 | ialwkvatal acgctvvlkp seetpltalr lgelaqeagl pdgvlnvvtg tgadtgaalv |
| 241 | ahpgvdkitf tgsvgvgkai ghaavdrmar ftlelggksp livfddadpd vaargaaqgi |
| 301 | ffnqgqvcta gsrvyvhksl fervvagiaa aaesmkigsg fdpntqigpl vskrhfervl |
| 361 | ghidaakeeg atlvtggtra ldggyfvkpt vfvdaapsmr ivreevfgpv vtvtpfdtvd |
| 421 | davrlandsd fglaasvwsq nlsrvhrvvp rlkagivwvn thnmldnnlp fggvkqsgyg |
| 481 | relgraaleq ftelksvcia h |

SEQ ID NO: 68
SeqID: YP_003978160.1 GI: 311105307
Protein name phenylacetaldehyde dehydrogenase
[Achromobacter xylosoxidans A8]

| | |
|---|---|
| 1 | mssasaiaag hagsdrpela aireamlidg kpvrvgqgap iavhdpatge viahqpdagp |
| 61 | lqvdlavqaa rrafesgpwr dmlpagrerl llkladlvel hgtelarlet lnngkllgva |
| 121 | qglevgsgaq wlrymagwtt kitgdtlsls ipfppgvrys aytlpqavgv vaaiipwnfp |
| 181 | llmaiwkiap alaagctvvl kpaeetplta lrlaelvlea gfppgvvnvv tgrgetagaa |
| 241 | lvahpgvdki aftgstevgk ligraamddm krvslelggk spvivlddcd vdravqgaaa |
| 301 | aiffnqgqvc tagsrlyvqr nlypkvvegl adlaagmrlg sgfdpatqvg plvsarhqkr |
| 361 | vmdyidigrn eggrvlaggg rgtgsgyfvq ptvfadvpsd ariareeifg pvvvaqpfdt |
| 421 | lddavrland sayglgaslw sndlsrvqsl iprvdagtvw vnthnmldpn mpfggfkqsg |
| 481 | igrehgravl emylerksvc iay |

SEQ ID NO: 69
SeqID: ZP_05974152.1 GI: 261346508
Protein name phenylacetaldehyde dehydrogenase
[Providencia rustigianii DSM 4541]

| | |
|---|---|
| 1 | mselallpev teflkrqhgh finglpvsgk gntyfdvvnp ateqviakvk egtlaevdaa |
| 61 | mdaaytafkg swanttpmer gnclsrladl lekhleelaq letlssgkti qlsrflevgs |
| 121 | aaqflryfag watkisgetl nvslpsfhge kysaftqrep vgvvagiipw nfsimisiwk |
| 181 | laaaltcgct ivlkpseftp ltmlrvaela keagipdgvi nivngggrev gpalihhslc |
| 241 | skvtftgsvp tglavgrsam eskltrvtle lggkngaafl adlpvdkivn giieagylnq |
| 301 | gqicaaaerf yipsklmddv ldalkvrlst mkigspldet temgplanka hydkilslfe |
| 361 | karqdgseii yggqpiagag yfvpptiira nspndilmqe etfgpvgtfl syddedelis |
| 421 | mmnstpfgla asvwtndlgk amrmvsqiea gtvwvnmhtf ldpavpfggi kssgigrefg |
| 481 | safiehytel ksvmiry |

SEQ ID NO: 70
SeqID: ZP_02361100.1 GI: 167568226
Protein name phenylacetaldehyde dehydrogenase
[Burkholderia oklahomensis C6786]

| | |
|---|---|
| 1 | mnladlstqh qrqsgflarr afgnwidgra aeprsgrylp vvdpatemti aevaasdard |
| 61 | vdaavaaarr afdsgdwprm rpanreklly glaelierha delaaletle tgklvgiara |
| 121 | idvlggaeyv rymagwatki egstldtsia vpadteyfay trreavgvvg aivpwnfpla |
| 181 | ialwkvatal acgctvvlkp seetpltalr lgelaqqagl pdgvlnivtg tggeagaalv |
| 241 | ahpgvdkitf tgsvgvgkai ghaavdrmar ftlelggksp liifddadpd vaargaaqgi |
| 301 | ffnqgqvcta gsrvyvqkrh fervvagiaa aaesmkigsg fdprtqigpl vskrhfervl |
| 361 | ghigaakteg atlvtggara fdggyfvkpt vfvdttpsmr ivreevfgpv vtvtpfdtvd |
| 421 | davrlandsd fglaasvwsq nlslvhrvvp rlkagivwvn thnmldnnlp fggfkqsgyg |
| 481 | relgraaleq ftelksvcia h |

SEQ ID NO: 71
SeqID: ZP_02361036.1 GI: 167568120
Protein name phenylacetaldehyde dehydrogenase
[Burkholderia oklahomensis EO147]

| | |
|---|---|
| 1 | mnladlstqh qrqsgflarr afgnwidgra aeprsgryfp vvdpatemti aevaasdard |
| 61 | vdaavaaarr afdsggwprm rpanreklly qlaelierha delaaletle tgklvgiara |
| 121 | idvlggaeyv rymagwatki egstldtsia vpadteyfay trreavgvvg aivpwnfpla |
| 181 | ialwkvatal acgctvvlkp seetpltalr lgelargagl pdgvlnivtg tggeagaalv |
| 241 | ahpgvdkitf tgsvgvgkai ghaavdrmar ftlelggksp liifddadpd vaargaaqgi |
| 301 | ffnqgqvcta gsrvyvqkrh fervvagiaa aaesmkigsg fdprtqigpl vskrhfervl |
| 361 | ghigaakaeg atlvtggara fdggyfvkpt vfvdttpsmr ivreevfgpv vtvtpfdtvd |
| 421 | davrlandsd fglaasvwsq nlslvhrvvp rlkagivwvn thnmldnnlp fggfkqsgyg |
| 481 | relgraaleq ftelksvcia h |

SEQ ID NO: 72
SeqID: ZP_06126412.1 GI: 268592191
Protein name phenylacetaldehyde dehydrogenase
[Providencia rettgeri DSM 1131]

| | |
|---|---|
| 1 | mseltllpev seflkrqhgh finglsysgk gdtffdvvnp ateqviakvk egtlaevdaa |
| 61 | mdaahtafkg vwanttpmer gnclnrladl lekhleelaq letlcsgkti qlsrflevgs |
| 121 | saqflryfag watkisgetl nvslpsfnge kysaftqrep vgvvagiipw nfsimisiwk |
| 181 | laaaltcgct ivlkpseftp ltmlrvvela keagvpdgvi nivngggrev gpalihhplc |
| 241 | skvtftgsvp tglavgrsam egkltrvtle lggkngaafl adlpvekivn giieagylnq |
| 301 | gqicaaaerf yipsklmdev laelktrlsa mkvgspldet temgplanka hyekilglfe |
| 361 | karqdgseii yggqpiagag yfvpptiira nspndvlmke etfgpigtfl syddeeelie |
| 421 | mmnstpfgla aslwtndlsk amrmisriea gtvwvnmhtf ldpavpfggv kssgigrefg |
| 481 | safieyytel ksvmvry |

SEQ ID NO: 73
SeqID: YP_003039308.1 GI: 253987952
Protein name phenylacetaldehyde dehydrogenase
[Photorhabdus asymbiotica]

| | |
|---|---|
| 1 | msditllqqv taflqrnhgh yingqsvhgq enqtfsvvnp avdeviatvn qggetevnaa |
| 61 | mqaahtafhg vwaqtspmer ghclnrladl llahreelaq leslcsgkti qlsrmleids |
| 121 | saqflryfag wsskisgetl nvslpsfkge qytaftrrep igvvvgiipw nfsimvaiwk |
| 181 | maaaltcgct ivlkpseytp ltmlrvaela keagipdgvi nvingsgsvl gpalighplc |
| 241 | akvtftgsvp tgitvgksam eqgltratle lggkngaafl admpvekivd gvleagylnq |
| 301 | gqicaaaerf yipashmdev lkllserlaa mkmgspldes temgplanke hynkilslfe |
| 361 | qargegseiv ygghaltgpg ffvaptvira nsaedslmke etfgpvgtff syndeeelie |
| 421 | lmnstpfgla aslwtndlsk amrmipriea gtvwvnmhtf ldpalpfggv kssgigrefg |
| 481 | safiehytel ksvmvry |

SEQ ID NO: 74
SeqID: YP_002438510.1 GI: 218889646
Protein name putative aldehyde dehydrogenase
[Pseudomonas aeruginosa LESB58]

| | |
|---|---|
| 1 | msiaidpsys aflrsphgll idgesgpars gadmplydpa tgaelarvar agaedvdrav |
| 61 | aaarrafegs wagqrpadre rlllrlaery eahgeqlaql etlnngksin lsralevgas |
| 121 | vefirymagw atkiegrsld lsiaavpgar yraytvpepv gvvgaivpwn fpllmaiwki |
| 181 | vpalacgctv vlkpadetpl talrlgqlcl eagippgvvn ivtgtgaeag aalaahpgid |
| 241 | klaftgstpv gklighaave nmtrfslelg gkspviildd tsldmaaags agaiffnqgq |
| 301 | vctagsrlyv qrkrfdqvle rlaaiagdls igpgldpttq inplvsarqq ervlgmiesg |
| 361 | vaegasvvcg garqgetgfy vqptiladvt pgmqvvreei fgpvlvatpf ddldeavrla |
| 421 | ndsiyglgas iwsndlrqvm dlvprikagt vwvnahnlld psmpfggfkq sgigremgha |
| 481 | aieaytenks vciay |

SEQ ID NO: 75
SeqID: ZP_04930284.1 GI: 254236961
Protein name hypothetical protein PACG_02985
[Pseudomonas aeruginosa C3719]

| | |
|---|---|
| 1 | msiaidpsvs aflrsphgll idgesgpars gadmplydpa tgtelarvar agaedvdrav |
| 61 | aaarrafegn wagqrpadre rlllrlaeri eahgeqlaql etlnngksin lsralevgas |
| 121 | vefirymagw atkiegrsld lsiaavpgar yraytvpepv gvvgaivpwn fpllmaiwki |
| 181 | vpalacgctv vlkpadetpl talrlgqlcl eagippgvvn ivtgtgaeag aalaahpgid |
| 241 | klaftgstpv gklighaave nmtrfslelg gkspviildd tsldmaaags agaiffnqgq |
| 301 | vctagsrlyv qrkrfdqvle rlaaiagdls igpgldpttq inplvsarqq ervlgmiesg |
| 361 | vaegasvvcg garqgetgfy vqptiladvt pgmqvvreei fgpvlvatpf ddldeavrla |
| 421 | ndsiyglgas iwsndlrqvm dlvprikagt vwvnahnlld psmpfggfkq sgigremgha |
| 481 | aieaytenks vciay |

SEQ ID NO: 76
SeqID: ZP_01367505.1 GI: 107103587
Protein name hypothetical protein PaerPA_01004657
[Pseudomonas aeruginosa PACS2]

| | |
|---|---|
| 1 | msiaidpsvs aflrsphgll idgesgpars gadmplydpa tgaelarvar agaedvdrav |
| 61 | aaarrafegn wagqrpadre rlllrlaery eahgeglaql etlnngksin lsralevgas |
| 121 | vefirymagw atkiegrsld lsiaavpgar yraytvpepv gvvgaivpwn fpllmaiwki |
| 181 | vpalacgctv vlkpadetpl talrlgqlcl eagippgvvn ivtgtgaeag aalaahpgid |
| 241 | klaftgstpv gklighaave nmtrfslelg gkspviildd tsldmaaags agaiffnqgq |
| 301 | vctagsrlyv qrkrfdqvle rlvaiagdls igpgldpttq inplvsarqq ervlgmiesg |
| 361 | vaegasvvcg garqgetgfy vqptiladvt pgmqvvreei fgpvlvatpf ddldeavrla |
| 421 | ndsiyglgas iwsndlrqvm dlvprikagt vwvnahnlld psmpfggfkq sgigremgha |
| 481 | aieaytenks vciay |

SEQ ID NO: 77
SeqID: NP_931461.1 GI: 37528116
Protein name phenylacetaldehyde dehydrogenase (PAD)
[Photorhabdus luminescens subsp. laumondii TTO1]

| | |
|---|---|
| 1 | msdinllqpv maflqhnhgh yingqpvsgq gsetfsvinp atdeiiatvn qggkaevnaa |
| 61 | mqaaqaafhg vwaqtspmer ghclnrladl llahreelaq letlcsgkti qlsrmleids |
| 121 | saqflryfag wsskisgetl nvslpsfkge qytaftrrep igvvvgiipw nfsimiaiwk |
| 181 | maaaltcgct ivlkpseytp ltmlrvaela kqagipdgvi nvingsgsvl gpalighplc |
| 241 | akvtftgsvp tgiavgksam eqgltratle lggkngaafl admsvekivd gileagylnq |
| 301 | gqicaaaerf yipashmddv lkllserlaa mkigspldds temgplanka hydkilslfe |
| 361 | qargegseiv ygghalagpg ffvaptvira nspedslmke etfgpvgtfl syndeeelig |
| 421 | lmnstpfgla aslwtndlsk amrmipriea gtvwvnmhtf ldpalpfggt kssgigrefg |
| 481 | safiehytel ksvmvry |

SEQ ID NO: 78
SeqID: NP_252762.1 GI: 15599268
Protein name aldehyde dehydrogenase
[Pseudomonas aeruginosa PAO1]

| | |
|---|---|
| 1 | msiaidpsys aflrsphgll idgesgpars gadmplydpa tgaelarvar agaedvdrav |
| 61 | aaarrafegn wagqrpadre rlllrlaeri eahgeqlaql etlnngksin lsralevgas |
| 121 | vefirymagw atkiegrsld lsiaavpgar yraytvpepv gvvgaivpwn fpllmaiwki |
| 181 | vpalacgctv vlkpadetpl talrlgqlcl eagippgvvn ivtgtgaeag aalaahpgid |
| 241 | klaftgstpv gklighaave nmtrfslelg gkspviildd tsldmaaags agaiffnqgq |
| 301 | vctagsrlyv qrkrfdqvle rlaaiagdls igpgldpttq inplvsarqq ervlgmiesg |
| 361 | vaegasvvcg garqgetgfy vqptiladvt pgmqvvreei fgpvlvatpf ddldeavrla |
| 421 | ndsiyglgas iwsndlrqvm dlvprikagt vwvnahnlld psmpfggfkq sgigremgha |
| 481 | aieaytenks vciay |

SEQ ID NO: 79
SeqID: YP_555177.1 GI: 91779969
Protein name putative phenylacetaldehyde dehydrogenase
[Burkholderia xenovorans LB400]

| | |
|---|---|
| 1 | madirilsev etflarqhrq yidgqavaam dtvatdiinp snrkvvasvr qatpaqvqha |
| 61 | vasaheaftg vwqqtsaarr gellnrladl mqahreelaq ieslssgkli glsrafeidy |
| 121 | siaflryyag watkihgqtm nlslpssnge qytgftlrqp igvvagivpw nfsimiavwk |
| 181 | fgsalacgct avikpseftp ltmlrvaela leagippgvl nivngngrtv gtaliahpkv |
| 241 | akvtftgsvp tgvgvgvaam qaglkhvtle lggknpagfl rdfpvertvd giieaaylhq |
| 301 | gevcasaerf yvhrsriddv lealhhtlat lkigsaldes aqfgplanaa hfakvmaffe |
| 361 | karaqdgeiv hggtvapgdg ffvqptaipa rsqadtimte etfgpvasfl ayddeeemlh |
| 421 | ymndthfglt asiwtndlsk alrfvprvea gtvwvnmhny idpampfggv kssgigrefg |
| 481 | eafieyftel ksvivry |

SEQ ID NO: 80
SeqID: YP_789047.1 GI: 116052109
Protein name putative aldehyde dehydrogenase
[Pseudomonas aeruginosa UCBPP-PA14]

| | |
|---|---|
| 1 | msiaidpsvs aflrsphgll iegesgpars gadmplydpa tgaelarvar agaedvdrav |
| 61 | aaarrafegn wagqrpadre rlllrlaeri eahgeglaql etlnngksin lsralevgas |
| 121 | vefirymagw atkiegrsld lsiaavpgar yraytvpepv gvvgaivpwn fpllmaiwki |
| 181 | vpalacgctv vlkpadetpl talrlgqlcl eagippgvvn ivtgtgaeag aalaahpgid |
| 241 | klaftgstpv gklighaave nmtrfslelg gkspviildd tsldmaaags agaiffnqgq |
| 301 | vctagsrlyv qrkrfdqvle rlaaiagdls igpgldpttq inplvsarqq ervlgmiesg |
| 361 | vaegasvvcg garqgetgfy vqptiladvt pgmqvvreei fgpvlvatpf ddldeavrla |
| 421 | ndsiyglgas iwsndlrqvm dlvprikagt vwvnahnlld psmpfggfkq sgigremgha |
| 481 | aieaytenks vciay |

SEQ ID NO: 81
SeqID: YP_164944.1 GI: 56708899
Protein name phenylacetaldehyde dehydrogenase
[Ruegeria pomeroyi DSS-3]

| | |
|---|---|
| 1 | mqvarpdslv sqilpavady ldspaklllg gtstaasdgr tmdvfnpatg kklaevpwgg |
| 61 | aaeidlavka aqaalegdws rmrpverqry llnladliea ngeelaqlet lnngksvmls |
| 121 | rlvevgnssn ylrymagwst kiegstidvs iavppgakyq aytrkepvgv vgaitpwnfp |
| 181 | lnmaiwklap alacgntvvl kpaeetplts lrlgelclea glppgvvnvv sgtgaeagaa |
| 241 | ltahpgvnkl tftgstevgk iigiqamrdm krvtlelggk apmvmfddmd ldqlseaari |
| 301 | gilfnsgqtc cagtriyaqr giydricetm anvvgalsvg sgldpanain pmvsakhqah |
| 361 | vsaciaggve egatplldtg aydgegyfvr pqiftdvrqd mrimqdevfg pvftitpfdd |
| 421 | pdeairmand tryglgasiw ttnlntmhry vpqlqagtvw vnshnvpdan mpfggykqsg |
| 481 | igrehgraal dayletksvc iavr |

SEQ ID NO: 82
SeqID: ZP_01166789.1 GI: 89093843
Protein name aldehyde dehydrogenase family protein
[Oceanospirillum sp. MED92]

| | |
|---|---|
| 1 | mstatfspse aaqaflqrqh kiligaewqa aqdgrtldvv npadgekiat vpsggaedid |
| 61 | ravsaakqaf edsewsrikp vdrqkllwdf adlieknapl laelealdng ksvviaehvd |
| 121 | irlavdflry magfatkieg rsvdvsvpfm pdaqfhgytr reavgvvgai vawnfpllla |
| 181 | cwklgpalat gctvvlkpae dtpltalkla elaleagypp gvfnvvtglg heagaalssh |
| 241 | pdvdkltftg stevgkligk aamdsmtrvt lelggkspti vlqdadlqna aagaanaiff |
| 301 | nqgqvccags rlyvhkkhfd nvvadisdia ngmtlgagld pnaqmgplvs akqqqrvcgy |
| 361 | idqgitsgak vvaggsaadg pgffvkptvm vdvdhnasvv keeifgpvlv ampfddidea |
| 421 | vriandsqyg lgasiwsnnl sevhrmipri ksgsvwvnch taldpalpfg gykqsglgre |
| 481 | mgsdviehyt evksvlmsi |

SEQ ID NO: 83
SeqID: ZP_02905263.1 GI: 171316036
Protein name Aldehyde Dehydrogenase_[Burkholderia
ambifaria MEX-5]

| | |
|---|---|
| 1 | malvrmldev raflarehgh yidgravagr geridvrdpa travigsvaq atdddveaai |
| 61 | asshrafrse wanrtpadre rillrfadli eahgeeiaqi etaqsgklig lsrvievgws |
| 121 | arwlryyagw atkiagetla psfpgmnger ytsftlrepl gvvfgiipwn fpvmipvwkf |
| 181 | gaalatgntv liksseftpl tmlriaelat eaglpagaln vingtgqvga kvigdprvak |
| 241 | vsftgsvptg riigeqavna nltrftlelg gknaaaflad tpvdkildgi veagflhsgq |
| 301 | vcasaerffv hrskfdevve kmkarldsfq padpmddagm igpvcnepqf rkcvdafdla |
| 361 | raegdtivtg ggayardgfy vkptivlprs lesasyrkei fgpvgafvpf ddeeeliami |
| 421 | ndtpfgltas lwtndlskal ryvprieagt vwvnmhtlvd pavpfggakg sgvgreygss |
| 481 | fidaytepka vtirf |

SEQ ID NO: 84
SeqID: YP_003450969.1 GI: 288960629
Protein name phenylacetaldehyde dehydrogenase
[Azospirillum sp. B510]

| | |
|---|---|
| 1 | mkiaqpdsiv eqqapavadf lkaplmviga asvpaksgrt ysvynpatgk plaevpagsa |
| 61 | edvdaavkaa qaafegpwsr mlptqrqaai lrladliean geelaqletl nngksimmsr |
| 121 | lleaqgaaey frymagwatk iegatldvsi pippgmsyqa ytrkepvgvv aaitpwnfpl |
| 181 | tmaawkvapa laagctvvlk paeetpltsi rlaqlcleag ipegvvnvvt glgeaagapl |
| 241 | vahpgiakis ftgstetgkl igiqamrdmk rvtlelggka pmvmfddmdl dllgvaagig |
| 301 | sffntgqtcc agvriyaqkg vydrvldtia avtrslsigs gldprnqinp lvsarhqahv |
| 361 | rsciargied gakpvikgsa padgfyvape lfvdvrqdma lmqeevfgpv vtvtpfddpd |
| 421 | eairlandtr fglgasiwtt dinkmmryvp kiqagtvwvn ahnlpdqnmp fggfkqsgvg |
| 481 | rehgrgaldn yletksvcva fr |

SEQ ID NO: 85
SeqID: YP_003713755.1 GI: 300724435
Protein name phenylacetaldehyde dehydrogenase
[Xenorhabdus nematophila ATCC 19061]

| | |
|---|---|
| 1 | mseinllgsv taflqrthgh yingvsvpgq gnetfsvvnp asgetiatvn qgeeadinqa |
| 61 | mqaasdafhg awartspler gnclnrladl lqengeelaq leslcsgkpi qlsrmlevga |
| 121 | sadylryfag wsskisgetl nvslpslkge kytaftrrep igvvvgiipw nfsimiaiwk |
| 181 | lgaalasgct ivlkpseytp liilrvaela keagipdgvi niingsgsry gsalishpqc |
| 241 | skvtftgsvp tgmivgksal eqglkhttle lggknaaafl sdmtvekivd gileagyvyq |
| 301 | gqicaaaerf yipsihmdav lellserlsa mkigspldes temgplankq hyekilslfe |
| 361 | qarqdgceiv yggyalegag ffvaptivra nspedtlmke etfgpigtfl syddeeelig |
| 421 | mmnstpfgls aslwtndlsk amrmipries gilwinmhty ldpsvpfggm kssgigrefg |
| 481 | safiehytel ksvmmry |

SEQ ID NO: 86
SeqID: YP_001057113.1 GI: 126441248
Protein name putative phenylacetaldehyde dehydrogenase
[Burkholderia pseudomallei 668]

| | |
|---|---|
| 1 | mnlaalstqh qrqsgflarr qfgnwidgra aeprsgrylp vvdpatemti aevaasdard |
| 61 | vdaavaaarr vfdsgdwprm rpasrekllh qlaerlerya delaaletle tgkligvara |
| 121 | idvlggaeyv rymagwatkl egstldtsia apagaeyfay trreavgvvg aivpwnfpla |
| 181 | ialwkvatal acgctvvlkp seetpltalr lgelaqeagl pdgvinivtg agaeagaala |
| 241 | ahpgidkitf tgsvgvgrai ghaavermar ftlelggksp livlddadpd faahgaaqgi |
| 301 | ffnqgqvcta gsrvyvqkrl fervvagiaa aaeamkigsg fdpntqigpl vskrhfervl |
| 361 | ghvdaakeeg atlvtggtra ldggyfvkpt vfvdaapamr ivreevfgpv vtvtpfdtvd |
| 421 | davrlandtd fglaasvwsq nlshvhrvvp rlkagivwvn thnmldpnlp fggfkcisgyg |
| 481 | relgraaleq ftelksvcia h |

SEQ ID NO: 87
SeqID: YP_104190.1 GI: 53723877
Protein name phenylacetaldehyde dehydrogenase
[Burkholderia mallei ATCC 23344]

| | |
|---|---|
| 1 | mnlaalstqh qrqsgflarr qfgnwidgra aeprsgrylp vvdpatemti aevaasdard |
| 61 | vdaavaaarr afdsgdwprm rpasrekllh qlaerlerya delaaletle tgkligvara |
| 121 | idvlggaeyv rymagwatkl egstldtsia apagaeyfay trreavgvvg aivpwnfpla |
| 181 | ialwkvatal acgctvvlkp seetpltalr lgelaqeagl pdgvlnivtg agaeagaala |
| 241 | ahpgidkitf tgsvgvgrai ghaavermar ftlelggksp livlddadpd faahgaaqgi |
| 301 | ffnqgqvcta gsrvyvqkrl fervvagiaa aaeamkigsg fdpntqigpl vskrhfervl |
| 361 | ghvdaakeeg atlvtggtra ldggyfvkpt vfvdaapamr ivreevfgpv vtvtpfdtvd |
| 421 | davrlandtd fglaasvwsq nlshvhrvvp rlkagivwvn thnmldpnlp fggfkqsgyg |
| 481 | relgraaleq ftelksvcia h |

SEQ ID NO: 88
SeqID: YP_776881.1 GI: 115359743
Protein name aldehyde dehydrogenase
[Burkholderia ambifaria AMMD]

| | |
|---|---|
| 1 | malvrmldev raflarehgh yidgravagr geridvrdpa travigsvaq atdddveaal |
| 61 | asshrafrge wanltpadre rillrfadli eahgeeiaqi etaqsgklig lsrvievgws |
| 121 | arwlryyagw atkiagetla psfpsmnger ytsftlrepl gvvfgiipwn fpvmipvwkf |
| 181 | gaalatgntv liksseftpl tmlriaelat eaglpagtln vingtgqvga kvigdprvak |
| 241 | vsftgsvptg riigeqavna nftrftlelg gknaaaflad tpvdkildgi veagflhsgq |
| 301 | vcasaerffv hrskfdevve kmkarldgfq padpmddagm igpvcnepqf rkcvdafdva |
| 361 | raegdtivtg ggayardgfy vkptivlprs lesasyrkei fgpvgafvpf ddeeeliamm |
| 421 | ndtpfgltas lwtndlskal ryvprieagt vwvnmhtivd pavpfggakg sgigreygss |
| 481 | fidaytepka vtirf |

SEQ ID NO: 89
SeqID: YP_001809829.1 GI: 172062178
Protein name aldehyde dehydrogenase
[Burkholderia ambifaria MC40-6]

| | |
|---|---|
| 1 | malvrmldev raflarehgh yidgravagr geridvrdpa travigsvaq atdddveaal |
| 61 | asshrafrge wadltpadre rillrfadli eahgeeiaqi etaqsgklia lsrvievgws |
| 121 | arwlryyagw atkiagetla psfpsmngeh ytsftlrepl gvvfgiipwn fpvmipvwkf |
| 181 | gaalatgntv liksseftpl tmlriaelat eaglpagtln vingtgqvga kvigdprvak |
| 241 | vsftgsvptg riigeqavna nftrftlelg gknaaaflad tpvdrildgi veagflhsgq |
| 301 | vcasaerffv hrskfdevve kmkarldafq padpmddagm igpvcnepqf rkcvdafdla |
| 361 | raegdtivtg ggayardgfy vkptivlprs lesasyrkei fgpvgafvpf ddeeeliamm |
| 421 | ndtpfgltas lwtndlskal ryvprieagt vwvnmhtlvd pavpfggakg sgigreygss |
| 481 | fidaytepka vtirf |

SEQ ID NO: 90
SeqID: YP_001669740.1 GI: 167034509
Protein name aldehyde dehydrogenase
[Pseudomonas putida GB-I]

| | |
|---|---|
| 1 | msditllpav taflarehgv fihgqhlasq ssstiavvnp angqtiahia danqadvdha |
| 61 | vsssrqgfat wshtspaara avlfkladll eahreelaql etvqsgklig israfeveqa |
| 121 | ahflryyagw atkitgqtit pslpsfager ysaftlrepi gvvvgivpwn fatmiaiwkl |
| 181 | asalttgcsi ilkpseftpl tllriaelat qaglpagaln vltggglvgk aliehagtdk |
| 241 | vsftgsvptg iavgqaamga kltratlelg gknavaflpd vatdkavdgi ieagflhsgq |
| 301 | icaagerfyv hrsridplld alsgrlgqlk igspldestq fgpvankphq qklaelfata |
| 361 | raegsqiihg gklgdgpgcf veptvilars andtllnget fgpvatflpy ddedellqlm |
| 421 | naspyglsas lwtndlgkam rmipgigagt lwvnmhtlld pavpfggika sgvgrefgsa |
| 481 | fiddftelks vmiry |

SEQ ID NO: 91
SeqID: YP_106676.1 GI: 53717690
Protein name putative phenylacetaldehyde dehydrogenase
[Burkholderi pseudomallei K96243]

| | |
|---|---|
| 1 | mnlaalstqh qrqsgflarr qfgnwidgra aeprsgcylp vvdpatemti aevaasdard |
| 61 | vdaavaaarr afdsgdwprm rpasrekllh qlaerlerya delaaletle tgkligvara |
| 121 | idvlggaeyv rymagwatkl egstldtsia apagaeyfay trreavgvvg aivpwnfpla |
| 181 | ialwkvatal acgctvvlkp seetpltalr lgelaqeagl pdgvinivtg agaeagaala |
| 241 | ahpgidkitf tgsvgvgrai ghaavermar ftlelggksp livlddadpd faahgaaqgi |
| 301 | ffnqgqvcta gsrvyvqkrl fervvagiaa aaeamkigsg fdpntqigpl vskrhfervl |
| 361 | ghvdaakeeg atlvtggtra ldggyfvkpt vfvdaapamr ivreevfgpv vtvtpfdtvd |
| 421 | davrlandtd fglaasvwsq nlshvhrvvp rlkagivwvn thnmldpnlp fggfkgsgyg |
| 481 | relgraaleq ftelksvcia h |

SEQ ID NO: 92
SeqID: ZP_02887976.1 GI: 170696880
Protein name Aldehyde Dehydrogenase
[Burkholderia ambifaria IOP40-10]

| | |
|---|---|
| 1 | malvrmldev raflarehgh yidgravagr geridvrdpa travigsvaq atdddveaal |
| 61 | asshrafrge wanltpadre rillrfadli eahgeeiaqi etaqsgklig lsrvievgws |
| 121 | arwlryyagw atkiagetla psfpsmnger ytsftlrepl gvvfgiipwn fpvmipvwkf |
| 181 | gaalatgntv liksseftpl tmlriaelat eaglpagtln vingagqvga kvirdprvak |
| 241 | vsftgsvptg riigeqavna nftrftlelg gknaaaflad tpvdkildgi veagflhsgq |
| 301 | vcasaerffv hrskfdevve kmkarldafq padpmddagm igpvcnepqf rkcvdafdla |
| 361 | rsegdtivtg ggayardgfy vkptivlprs lesasyrkei fgpvgafvpf ddeeqliamm |
| 421 | ndtpfgltas lwtndlskal ryvprieagt vwvnmhtivd pavpfggakg sgigreygss |
| 481 | fidaytepka vtirf |

SEQ ID NO: 93
SeqID: ZP_05812654.1 GI: 260465464
Protein name Aldehyde Dehydrogenase
[Mesorhizobium opportunistum WSM2075]

| | |
|---|---|
| 1 | mnertlppis paaaaflarp hrpfingrfv dglagdglav ddsasgeiva hvpesgpelv |
| 61 | dqavraaraa legpwasmrp vdrqnlmlkl adaveadadl laeiesieng kslgvarmls |
| 121 | aagtvdwlry yagwatkieg stfqvsipvp pgakhqamtv mepvgvvgai vpwnfpllig |
| 181 | mwkiapalac gctvvlkppq etplgllrla elieasgfpp gvvnivtgsg svtgealirh |
| 241 | pgidkltftg stevgkrvgh aavdrvarft lelgskspmi lladmeegie plvaglgmff |
| 301 | nqgqvctsas rllieksiyd rtlarlaeia dgmtlgagrd adaqinplvs akhkrsvegf |
| 361 | verglaagve rvsgarpvpa rghyvaptil hnvrpdmeiv reevfgpvva ampvadldea |
| 421 | iriandtryg lsasiwtrdm gkamtaihgl kagtvwvnsh ntldpnapfg gfkgsgigre |
| 481 | hgraaidgyl etktvimrya |

SEQ ID NO: 94
SeqID: YP_590457.1 GI: 94968409
Protein name aldehyde dehydrogenase (acceptor)
[Candidatus Koribacter versatilis Ellin345]

| | |
|---|---|
| 1 | msvvsaveln snvsqfitkp rkmliggnwi dsasgkffet lnpatgevla rvaegdradi |
| 61 | dlavaaarka fesgpwskms psqrgrllwk ladlleqhle efaelesldn gkplsvarva |
| 121 | dvplavdlfr ymagwatkve gntiplgpqf haytyrepvg vigqiipwnf pllmaawklg |
| 181 | palavgctvv lkpaeqtpls alrlgelime agfpdgvvnv vpgfgetaga alaahpdvdk |
| 241 | iaftgstevg klivqaaagn lkkvslelgg kspnivlada dldiaisgsa naiffnhgqc |
| 301 | ccagsrlfvh ksqfdkvveg vaeaaknirl gsgldpatnm gplvsqeqld rvcgylesgv |
| 361 | qqgakplvgg kkqtgpgyfv eptvlvdvkp tmkvvceeif gpvvtaipfn svdevlnsan |
| 421 | assyglaaav wtrdinkahs laaklragtv wvncynvfda alpfggykqs gwgremghda |
| 481 | lelytetkav cvrlen |

SEQ ID NO: 95
SeqID: YP_001027814.1 GI: 124384294
Protein name phenylacetaldehyde dehydrogenase
[Burkholderia mallei NCTC 10229]

| | |
|---|---|
| 1 | mnlaalstqh qrqsgflarr qfgnwidgra aeprlgrylp vvdpatemti aevaasdard |
| 61 | vdaavaaarr afdsgdwprm rpasrekllh qlaerlerya delaaletle tgkligvara |
| 121 | idvlggaeyv rymagwatkl egstldtsia apagaeyfay trreavgvvg aivpwnfpla |
| 181 | ialwkvatal acgctvvlkp seetpltalr lgelaqeagl pdgvlnivtg agaeagaala |
| 241 | ahpgidkitf tgsvgvgrai ghaavermar ftlelggksp livlddadpd faahgaaqgi |
| 301 | ffnqgqvcta gsrvyvqkrl fervvagiaa aaeamkigsg fdpntqigpl vskrhfervl |
| 361 | ghvdaakeeg atlvtggtra ldggyfvkpt vfvdaapamr ivreevfgpv vtvtpfdtvd |
| 421 | davrlandtd fglaasvwsq nlshvhrvvp rlkagivwvn thnmldpnlp fggfkqsgyg |
| 481 | relgraaleq ftelksvcia h |

SEQ ID NO: 96
SeqID: YP_001583562.1 GI: 161520135
Protein name aldehyde dehydrogenase
[Burkholderia multivorans ATCC 17616]

| | |
|---|---|
| 1 | metndnfall datraflakp krmliggews daasgrtiev vnpadgstia cvpeadehdv |
| 61 | qravaaarra fdtgpwraak ttdrerlllt ladlidanar elaeiesldn gksysvaqgl |
| 121 | dvamaaqcfr ymagwatkie gsvvdagmpy lpgsetftyt rkepigvvga iipwnfpllm |
| 181 | aawklapala tgctvvlkpa edtpltalrl geligdagfp dgvvnivtgy ghtagaalsr |
| 241 | dpridkiaft gstqtgkaig haaldnmtrm slelggkspv ivlpdvdvdk aaegianaif |
| 301 | fnqgqvctag srayvhtkvf drvmervaqi aaglkigpgm dpatqigplv sakqrarvcd |
| 361 | yiasgfedga raiaggrard gagffveptv lvdtthamry vreeifgpvl vampfddidt |
| 421 | avqlandtpy glgasiwsnd lsavhklvpr iaagtvwvnc hslldnampf ggmkqsgfgr |
| 481 | elgravidqy tetksvmmny a |

SEQ ID NO: 97
SeqID: YP_331688.1 GI: 76809054
Protein name phenylacetaldehyde dehydrogenase
[Burkholderia pseudomallei 1710b]

| | |
|---|---|
| 1 | mnlaalstqh qrqsgflarr qfgnwidgra aeprsgrylp vvdpatemti aevaasdard |
| 61 | vdaavaaarr afdsgdwprm rpasrekllh qlaerlerya delaaletle tgkligvara |
| 121 | idvlggaeyv rymagwatkl egstldtsia apagaeyfay trreavgvvg aivpwnfpla |
| 181 | ialwkvatal acgctvvlkp seetpltalr lgelaqeagl pdgvlnivtg agaeagaala |
| 241 | ahpgidkitf tgsvgvgrai ghaavermar ftlelggksp livlddadpd faahgaaqgi |
| 301 | ffnqgqvcta gsrvyvqkrl fervvagiaa aaeamkigsg fdpntqigpl vskrhfervl |
| 361 | ghvgaakeeg atlvtggtra ldggyfvkpt vfvdaapamr ivreevfgpv vtvtpfdtvd |
| 421 | davrlandtd fglaasvwsq nlshvhrvvp rlkagivwvn thnmldpnlp fggfkqsgyg |
| 481 | relgraaleq ftelksvcia h |

SEQ ID NO: 98
SeqID: ZP_01768110.1 GI: 134281402
Protein name phenylacetaldehyde dehydrogenase
[Burkholderia pseudomallei 305]

| | |
|---|---|
| 1 | mnlaalstqh qrqsgflarr qfgnwidgra aeprsgrylp vvdpatemti aevaasdard |
| 61 | vdaavaaarr afdsgdwprm rpasrekllh qlaerlerya delaaletle tgkligvara |
| 121 | idvlggaeyv rymagwatkl egstldtsia apagaeyfay trreavgvvg aivpwnfpla |
| 181 | ialwkvatal acgctvvlkp seetpltalr lgelareagl pdgvlnivtg agaeagaala |
| 241 | ahpgidkitf tgsvgvgrai ghaavermar ftlelggksp livlddadpd faahgaaqgi |
| 301 | ffnqgqvcta gsrvyvqkrl fervvagiaa aaeamkigsg fdpntqigpl vskrhfervl |
| 361 | ghvgaakeeg atlvtggtra ldggyfvkpt vfvdaapamr ivreevfgpv vtvtpfdtvd |
| 421 | davrlandtd fglaasvwsq nlshvhrvvp rlkagivwvn thnmldpnlp fggfkqsgyg |
| 481 | relgraaleq ftelksvcia h |

SEQ ID NO: 99
SeqID: YP_001342862.1 GI: 152998027
Protein name aldehyde dehydrogenase
[Marinomonas sp. MWYL1]

| | |
|---|---|
| 1 | msdiplldst qrflqqdhgq fingqtkasg dntfdiinps teaviakihs attqevdaai |
| 61 | essyqvfkga wgktspyirg vvlnkladli eqngeeiaql etlcsgksih lsrmfevqqs |
| 121 | amflryfagw stkingetmt psfpsmqgee ysaftrreai gvvagilpwn fsvmiacwki |
| 181 | gaalctgcti vlkpseftpl tilriaelak eagvpdgvin ivngkgdvgg qliqhpkvrk |
| 241 | vsftgsvatg kkisaaasad ltrctlelgg kntaailkda didrvvgglf qlgyihqgqv |
| 301 | caapervyvh ssridelttk laqklseaki gspldesvyf gplsnepqfn kvceyleiah |
| 361 | kesrvlhggk aisgkgffve ptivqassvd etimqeetfg piisfmpyed eeelidlinn |
| 421 | tpfglsssiw tnnlsqamrm ipkiesgtvw vnmhsildps vpfggtkqsg vgrefgrefi |
| 481 | ndytevksvi mcy |

SEQ ID NO: 100
SeqID: ZP_03582393.1 GI: 221209412
Protein name phenylacetaldehyde dehydrogenase (PAD)
[Burkholderia multivorans CGD1]

| | |
|---|---|
| 1 | metndnfall datraflakp krmliggews daasgrtiev vnpadgstia cvpeadqhdv |
| 61 | qravaaarra fdagpwraak ttdrerlllt ladlidanar elaevesldn gksvivaqgl |
| 121 | dvamaaqcfr ymagwatkie gsvvdagmpy lpgsetftyt rkepvgvvga iipwnfpllm |
| 181 | aawklapala tgctvvlkpa edtpltalrl geligeagfp dgvvnivtgy ghtagaalsr |
| 241 | dpridkiaft gstqtgkaig haaldnmtrm slelggkspv ivlpdvdvdk aaegianaif |
| 301 | fnqgqvctag srayvhtkvf drvmervaqi aaglkigpgm dpatqigplv sakqrarvcd |
| 361 | yiasgfedga raiaggrard gagffveptv lvdtthamrv vreeifgpvl vampfddidt |
| 421 | avqlandtpy glgasiwsnd lsavhklvpr iaagtvwvnc hslldnampf ggmkqsgfgr |
| 481 | elgravidqy tetksvmmny a |

SEQ ID NO: 101
SeqID: ZP_03573421.1 GI: 221200379
Protein name phenylacetaldehyde dehydrogenase (PAD)
[Burkholderia multivorans CGD2M]

| | |
|---|---|
| 1 | metndnfall datraflakp krmliggews daasgrtiev vnpadgstla rvpeadehdv |
| 61 | qravaaarra fdtgpwraak ttdrerlllt ladlidanar elaeiesldn gksvsvaqgl |
| 121 | dvamaaqcfr ymagwatkie gsvidagmpy lpgsetftyt rkepvgvvga iipwnfpllm |
| 181 | aawklapala tgctvvlkpa edtpltalrl geligdagfp dgvvnivtgy ghtagaalsr |
| 241 | dpridkiaft gstqtgkaig haaldnmtrm slelggkspv ivladvdvdk aaegianaif |
| 301 | fnqgqvctag srayvhtkvf drvmeraaqi aaglkigpgm dpatqigplv sakqrarvcd |
| 361 | yiasgfeega raiaggrard gagffveptv lvdtthamrv vreeifgpvl vampfddidt |
| 421 | avrlandtpy glgasiwsnd lsavhklvpr iaagtvwvnc hslldnampf ggmkqsgfgr |
| 481 | elgravidqy tetksvmmny a |

SEQ ID NO: 102
SeqID: YP_001346414.1 GI: 152984537
Protein name putative aldehyde dehydrogenase
[Pseudomonas aeruginosa PA7]

| | |
|---|---|
| 1 | msiaidpsvt aflrshhgll idgesrpars gadmplydpa sgaelarvar aaaddvdlav |
| 61 | aaarrafegs waqqrpadre rlllclaerl eahgeqlaql etlnngksin lsralevgas |
| 121 | vefirymagw atkiegrsld lsiaavpgar yraytvpepv gvvgaivpwn fpllmavwki |
| 181 | vpalacgctv vlkpadetpl talrlgqlcl eagippgvvn ivtgtgaeag aalaahpgid |
| 241 | klaftgstpv gklighaave nmtrfslelg gkspviildd tsldmaaags aaaiffnqgq |
| 301 | vctagsrlyv qrkrfeqvle rlasiaadln igpgldpaaq inplvsarqq grvlgmiegg |
| 361 | vaegarvvcg garagetgfy vqptvladvt prmqvvreei fgpvlvatpf ddldeavrla |
| 421 | ndsiyglgas iwsndlrqvm dllprikagt vwvnthnmld psmpfggfkq sgigremgha |
| 481 | aieaytenks vciay |

SEQ ID NO: 103
SeqID: ZP_06465644.1 GI: 289633355
Protein name NAD-dependent aldehyde dehydrogenase
[Burkholderia sp. CCGE1003]

| | |
|---|---|
| 1 | mndnsrpldm ldstrtflaa pkrmfidgew rasasgatld vlnpadgsll aqvpsadead |
| 61 | vdlavqaarr afddsawsrm kptdrerill rvaelieana relaeiesld ngkpvavaqg |
| 121 | ldvsmaaqcf rymagwatki egstldaalp yspsnaffay trkeavgvvg aiipwnfpll |
| 181 | maswklapal atgctvvlkp aedtplsalr latllseagl pkgvvnivtg ygrsagaala |
| 241 | rhpgidkiaf tgstqtgkai ghaaldnmtr mslelggksp vivlpdvdie raaegvanai |
| 301 | ffnsgqvcta gsrvyvhetv fdrvmervaa iaealpvgpg ldantqigpl vsarqmdrvl |
| 361 | gyieagrdeg araiaggare ggagffvkpt vlvdtdhsmr vvreeifgpv lvampfkdid |
| 421 | savaqandtp yglgasiwsn nlsaihnlip rikagtvwvn chslldnamp fggvkqsgfg |
| 481 | relgravidm ytemksvlin ha |

SEQ ID NO: 104
SeqID: ZP_06690265.1 GI: 293607962
Protein name conserved hypothetical protein
[Acinetobacter sp. SH024]

| | |
|---|---|
| 1 | msevqilesv qqfmarqhgh fidgklvaae lldkvdivnp steevvaqis igsqqdvdsa |
| 61 | vksaehafqn awaettpyer gvklnkladl ieqygeelaq letlstgkli nisrhlevaq |
| 121 | sviflryfag watkingqtm qpsipsmqge kytaftlrqp igvvagivpw nfslmigvwk |
| 181 | igsalttgct ivlkpsefas lsllrlaela ieagipvgvi nvvtgkgetg qyliesplvk |
| 241 | kvsftgsvpt giaigklams sdltrvslel ggknaiavla danideilpt llqatfvhqg |
| 301 | qvcasperff vhrakydelv eklskalssf kigsamdegs mfgplsnqph fhkvkhyldm |
| 361 | akaknqiiag getldrsgyf vqptlisfkn tddplfseet fgpvvgimpf etdeelvqlm |
| 421 | nqsrfgltas iwtndlskal rlipkieagt lwvnmhtfld psvpfggvka sgigrefsda |
| 481 | fiedytelks vmiry |

SEQ ID NO: 105
SeqID: ZP_05824478.1 GI: 260550266
Protein name phenylacetaldehyde dehydrogenase
[Acinetobacter sp. RUH2624]

| | |
|---|---|
| 1 | msevqilqnv qqfmarqhgh fidgklvaae hldkvdivnp steqvvaqis igsqqdvasa |
| 61 | vksakhafqn awaettpyer gvklnkladl ieqhgeelaq letlstgkli nisrhlevaq |
| 121 | sviflryfag watkingqtm qpsipsmqge kytaftlrqp vgvvagivpw nfslmigiwk |
| 181 | igsalttgct ivlkpsefas lsllrlaela ieagipagvi nvvtgkgdtg qyliesplvk |
| 241 | kvsftgsvpt giaigklams sdltrvslel ggknaiavla danideilpt llqatfvhqg |
| 301 | qvcasperff vhhtkhnelv eklskalssl kigsamdegs mfgplsnqph fhkvkhyldm |
| 361 | akannqiiag gealdrsgyf vqptlisfkn tddplfseet fgpvvgvmpf etdeeliqlm |
| 421 | nqsrfgltas iwtndlskal rlipkieagt lwvnmhtfld psvpfggvka sgigrefsda |
| 481 | fiedytelks vmiry |

SEQ ID NO: 106
SeqID: ZP_04663661.1 GI: 239504351
Protein name NAD-dependent aldehyde dehydrogenase
[Acinetobacter baumanni AB900]

| | |
|---|---|
| 1 | msevqilesv qqfiarqhgh fidgklvaae lldkvdivnp steqvvaqis igsqqdvesa |
| 61 | vksaehafqn awaettpyer gvklnkladl ieqhgeelaq letlstgkli nisrhlevaq |
| 121 | sviflryfag watkingqtm qpsipsmqge kytaftlrqp vgvvagivpw nfslmigvwk |
| 181 | igsalttgct ivlkpsefas lsllrlaela ieagipagvi nvvtgkgetg qyliesplvk |
| 241 | kvsftgsvpt giaigklams sdltrvslel ggknaiavla danideilpt llqatfvhqg |
| 301 | qvcasperfl vhrtkydelv dklskalsqf kigsamdegs mfgplsnqph fhkvkhyldm |
| 361 | akannqiiag gealdqtgyf vqptlisfkn tddplfseet fgpvvgvmsf dtdeeliqlm |
| 421 | nqsrfgltas iwtndlskal rlipkieagt lwvnmhtfld psvpfggvka sgigrefsda |
| 481 | fiedytelks vmiry |

SEQ ID NO: 107
ATGATCGCTAGCAGGAGAAATTAACT ATGTTGACAAAAGCAACAAAAGAACA
SEQ ID NO: 108
GACTATGCTCAGCTTAGAGAGCTTTCGTTTTCATGAGTTC
SEQ ID NO: 109
GACTATGCTCAGCTTAGAGAGCTTTCGTTTTCATGAGTTC
SEQ ID NO: 110
TCCTCTAAATCTCTAGAAAGGGTGCCGGCAGCTTGATATGTT
SEQ ID NO: 111
ATGATCGGTACCATGCCTAAGTACCGTTCCGCCA
SEQ ID NO: 112
ATGATCGCTAGCTTAACCCCCCAGTTTCGATTTATCG
SEQ ID NO: 113
GACTATGGTACCATGTATACAGTAGGAGATTACCTATTAG
SEQ ID NO: 114
GACTATGCATGCTTATGATTTATTTTGTTCAGCAAATAG
SEQ ID NO: 115
GACTATTCTAGATTATGATTTATTTTGTTCAGCAAATAG
SEQ ID NO: 116
GACTATGCATGCAGGAGATATACC ATGCAACATAAGTTACTGATTAACGGAG
SEQ ID NO: 117
GACTATTCTAGATTAATGTTTAACCATGACGTGGCGGACG
SEQ ID NO: 118
GACTATGCATGCAGGAGATATACCATGACCAATAATCCCCCTTCAGCA
SEQ ID NO: 119
GACTATTCTAGATTAGAACAGCCCCAACGGTTTATCCGA
SEQ ID NO: 120
GACTATGCATGCAGGAGATATACCATGAATTTTCATCATCTGGCTTACTGGCA
SEQ ID NO: 121
GGCTTATCCAGATGGTTTTCAGTTCAGTGAATTTTTCAAGGGCGTGCAGGGATTTGTCGC
SEQ ID NO: 122
GACTATTCTAGATTAGGCCTCCAGGCTTATCCAGATGGTTTTCAGTTCAG
SEQ ID NO: 123
GACTATGCATGCAGGAGATATACCATGAAACTTAACGACAGTAACTTATTCC
SEQ ID NO: 124
GACTATTCTAGATTAAAGACCGATGCACATATATTTGA
SEQ ID NO: 125
CTAGTAGCATGCAAGGAGATATACC ATGACAGAGCCGCATGTAGCAGT
SEQ ID NO: 126
GACTATTCTAGATTAATACCGTACACACACCGACTTAGTT
SEQ ID NO: 127
GACTATGCATGCAGGAGATATACC ATGGCTAACGTGACTTATACGGATACG
SEQ ID NO: 128
GACTATTCTAGATTAGACCGCCATCACCGTCACC
SEQ ID NO: 129
GACTATGCATGCAGGAGATATACCATGCCCCTCACAGGCAACCTG
SEQ ID NO: 130
GACTATTCTAGATTAGTCTTCCCGTTTACCATCAAGCA
SEQ ID NO: 131
GACTATGGATCCATGACAGAGCCGCATGTAGCAGT
SEQ ID NO: 132
GACTATGGATCCTTAATACCGTACACACACCGACTTAGTT

1. A recombinant microbial cell modified to exhibit increased biosynthesis of isobutylc acid compared to a wild type control.

2-70. (canceled)
